(19) United States
(12) Patent Application Publication
(10) Pub. No.: US 2024/0360101 A1
(43) Pub. Date: Oct. 31, 2024

(54) PHARMACEUTICAL SALTS OF A CHK-1 INHIBITOR

(71) Applicants: SENTINEL ONCOLOGY LIMITED, Cambridge (GB); PHARMAENGINE, INC., Taipei (TW)

(72) Inventors: Meriel MAJOR, Cambridge (GB); Stuart TRAVERS, Shefford (GB); Jake PARKER, Sunderland (GB); Julian NORTHEN, South Shields (GB)

(73) Assignees: SENTINEL ONCOLOGY LIMITED, Cambridge (GB); PHARMAENGINE, INC., Taipei (TW)

(21) Appl. No.: 18/566,777

(22) PCT Filed: Jun. 1, 2022
(86) PCT No.: PCT/EP2022/064935

§ 371 Date: Dec. 4, 2023

(30) Foreign Application Priority Data
Jun. 3, 2021 (GB) 2107924.9

(51) Int. Cl. C07D 401/14; A61K 9/00; A61K 9/19; A61K 9/20; A61K 9/48; A61K 31/497; A61K 47/10; A61K 47/26; A61K 47/44; C07C 55/08; C07C 309/04; C07C 309/29; C07C 309/30

(52) U.S. Cl. CPC C07D 401/14; A61K 9/0019; A61K 9/19; A61K 9/2013; A61K 9/2018; A61K 9/4825; A61K 9/4858; A61K 31/497; A61K 47/10; A61K 47/26; A61K 47/44; C07C 55/08; C07C 309/04; C07C 309/29; C07C 309/30; C07B 2200/13

(57) ABSTRACT

The invention provides a pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile which is selected from maleate, tosylate, besylate and malonate salts.

Also provided are particular crystalline forms of the salts, methods for the preparation of the salts, pharmaceutical compositions containing the salts and their therapeutic uses.

This invention relates to pharmaceutical salts of the Chk-1 inhibitor compound 5-[[5-[4-(4-fluoro-1-methyl-4-piperidyl)-2-methoxy-phenyl]-1H-pyrazol-3-yl]amino]pyrazine-2-carbonitrile, methods for their preparation, pharmaceutical compositions containing them and their uses in treating diseases such as cancer.

### BACKGROUND OF THE INVENTION

Chk-1 is a serine/threonine kinase involved in the induction of cell cycle checkpoints in response to DNA damage and replicative stress [Tse et al, Clin. Can. Res. 2007; 13(7)]. Cell cycle checkpoints are regulatory pathways that control the order and timing of cell cycle transitions. Many cancer cells have impaired G1 checkpoint activation. For example, Hahn et al., and Hollstein et al., have reported that tumours are associated with mutations in the p53 gene, a tumour suppressor gene found in about 50% of all human cancers [N Engl J Med 2002, 347(20):1593; Science, 1991, 253(5015):49].

Chk-1 inhibition abrogates the intra S and G2/M checkpoints and has been shown to selectively sensitise tumour cells to well known DNA damaging agents. Examples of DNA damaging agents where this sensitising effect has been demonstrated include Gemcitabine, Pemetrexed, Cytarabine, Irinotecan, Camptothecin, Cisplatin, Carboplatin [Clin. Cancer Res. 2010, 16, 376], Temozolomide [Journal of Neurosurgery 2004, 100, 1060], Doxorubicin [Bioorg. Med. Chem. Lett. 2006; 16:421-6], Paclitaxel [WO2010149394], Hydroxy urea [Nat. Cell. Biol. 2005; 7(2):195-20], the nitroimidazole hypoxia-targeted drug TH-302 (Meng et al., AACR, 2013 Abstract No. 2389) and ionising radiation [Clin. Cancer Res. 2010, 16, 2076]. See also the review article by McNeely et al., [Pharmacology & Therapeutics (2014), 142(1):1-10]

Recently published data have also shown that Chk-1 inhibitors may act synergistically with PARP inhibitors [Cancer Res 2006; 66:(16)], Mek inhibitors [Blood. 2008; 112(6): 2439-2449], Farnesyltransferase inhibitors [Blood. 2005; 105(4):1706-16], Rapamycin [Mol. Cancer Ther. 2005; 4(3):457-70], Src inhibitors [Blood. 2011; 117(6):1947-57] and WEE1 inhibitors [Carrassa, 2021, 11(13):2507; Chaudhuri et al., Haematologica, 2014 99(4):688.].

Furthermore, Chk-1 inhibitors have demonstrated an advantage when combined with immunotherapy agents [Mouw et al., Br J Cancer, 2018. (7):933]. Chk1 inhibitors have been shown to activate cGAS, which induces an innate immune response through STING signaling, and to induce PD-L1 expression and synergize with anti-PD-L1 in vivo [Sen et al., Cancer Discov 2019 (5):646; Sen et al., J Thorac Oncol, 2019. (12):2152].

Resistance to chemotherapy and radiotherapy, a clinical problem for conventional therapy, has been associated with activation of the DNA damage response in which Chk-1 has been implicated [Nature; 2006; 444(7):756-760; Biochen. Biophys. Res. Commun. 2011; 406(1):53-8].

It is also envisaged that Chk-1 inhibitors, either as single agents or in combination, may be useful in treating tumour cells in which constitutive activation of DNA damage and checkpoint pathways drive genomic instability in particular through replication stress. This phenotype is associated with complex karyotypes, for example in samples from patients with acute myeloid leukemia (AML) [Cancer Research 2009, 89, 8652]. In vitro antagonisation of the Chk-1 kinase with a small molecule inhibitor or by RNA interference strongly reduces the clonogenic properties of high-DNA damage level AML samples. In contrast Chk-1 inhibition has no effect on normal hematopoietic progenitors. Furthermore, recent studies have shown that the tumour microenvironment drives genetic instability [Nature; 2008; (8):180-192] and loss of Chk-1 sensitises cells to hypoxia/reoxygenation [Cell Cycle; 2010; 9(13):2502]. In neuroblastoma, a kinome RNA interference screen demonstrated that loss of Chk-1 inhibited the growth of eight neuroblastoma cell lines. Tumour cells deficient in Fanconi anemia DNA repair have shown sensitivity to Chk-1 inhibition [Molecular Cancer 2009, 8:24]. It has been shown that the Chk-1 specific inhibitor PF-00477736 inhibits the growth of thirty ovarian cancer cell lines [Bukczynska et al, 23^{rd} Lorne Cancer Conference] and triple negative breast cancer cells [Cancer Science 2011, 102, 882]. Also, PF-00477736 has displayed selective single agent activity in a MYC oncogene driven murine spontaneous cancer model [Ferrao et al, Oncogene (15 Aug. 2011)]. Chk-1 inhibition, by either RNA interference or selective small molecule inhibitors, results in apoptosis of MYC-overexpressing cells both in vitro and in an in vivo mouse model of B-cell lymphoma [Höglund et al., Clinical Cancer Research, 2011]. The latter data suggest that Chk-1 inhibitors would have utility for the treatment of MYC-driven malignancies such as B-cell lymphoma/leukemia, neuroblastoma and some breast and lung cancers. Chk-1 inhibitors have also been shown to be effective in paediatric tumour models, including Ewing's sarcoma and rhabdomyosarcoma [Lowery, 2018. Clin Cancer Res 2019, 25(7):2278]. Chk1 inhibitors have been shown to be synthetically lethal with the B-family of DNA polymerases, resulting in increased replication stress, DNA damage and cell death [Rogers et al., 2020, 80(8); 1735]. Other cell cycle regulated genes have also been reported to confer sensitivity to Chk-1 inhibitors, including CDK2 and POXM1 [Ditano et al., 20201. 11(1); 7077; Branigan et al., 2021 Cell Reports 34(9):1098808]

It has also been reported that mutations that reduce the activity of DNA repair pathways can result in synthetically lethal interactions with Chk1 inhibition. For example, mutations that disrupt the RAD50 complex and ATM signaling increase responsiveness to Chk1 inhibition [AI-Ahmadie et al., Cancer Discov. 2014. (9):1014-21]. Likewise, deficiencies in the Fanconi anemia homologous DNA repair pathway lead to sensitivity to Chk1 inhibition [Chen et al., Mol. Cancer 2009 8:24, Duan et al., Frontiers in Oncology 2014 4:368]. Also, human cells that have loss of function in the Rad17 gene product are sensitive to Chk1 suppression [Shen et al., Oncotarget, 2015. 6(34):35755].

Various attempts have been made to develop inhibitors of Chk-1 kinase. For example, WO 03/10444 and WO 2005/072733 (both in the name of Millennium) disclose aryl/heteroaryl urea compounds as Chk-1 kinase inhibitors. US2005/215556 (Abbott) discloses macrocyclic ureas as kinase inhibitors. WO 02/070494, WO2006014359 and WO2006021002 (all in the name of Icos) disclose aryl and heteroaryl ureas as Chk-1 inhibitors. WO/2011/141716 and WO/2013/072502 both disclose substituted pyrazinyl-phenyl ureas as Chk-1 kinase inhibitors. WO2005/009435 (Pfizer) and WO2010/077758 (Eli Lilly) disclose aminopyrazoles as Chk-1 kinase inhibitors

WO2015/120390 discloses a class of substituted phenyl-pyrazolyl-amines as Chk-1 kinase inhibitors. One of the compounds disclosed is the compound 5-[[5-[4-(4-fluoro-1-methyl-4-piperidyl)-2-methoxy-phenyl]-1H-pyrazol-3-yl]amino]pyrazine-2-carbonitrile, the synthesis of which is described in Example 64 and Synthetic Method L in WO2015/12039. The compound is disclosed in the form of its hydrochloride salt.

WO2018/183891 (Cascadian Therapeutics) discloses combinations of the compound 5-[[5-[4-(4-fluoro-1-methyl-4-piperidyl)-2-methoxy-phenyl]-1H-pyrazol-3-yl]amino]pyrazine-2-carbonitrile or a pharmaceutically acceptable salt thereof with WEE-1 inhibitors. However, no specific salts of 5-[[5-[4-(4-fluoro-1-methyl-4-piperidyl)-2-methoxy-phenyl]-1H-pyrazol-3-yl]amino]pyrazine-2-carbonitrile are disclosed.

### The Invention

It has now been found that 5-[[5-[4-(4-fluoro-1-methyl-4-piperidyl)-2-methoxy-phenyl]-1H-pyrazol-3-yl]amino]pyrazine-2-carbonitrile forms crystalline salts with a number of mineral acids and organic acids.

Accordingly, in a first aspect (Embodiment 1.1), the invention provides a pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile which is selected from hydrobromide, mesylate, L-tartrate, esylate, L-aspartate, besylate, tosylate, sulphate, phosphate, citrate, acetate, L-glutamate, maleate, gentisate, glucuronate, malonate, naphthylene-2-sulphonate, ethane-1,2-disulphonate, naphthalene-1,5-disulphonate and oxalate salts.

The terms “hydrobromide, mesylate, L-tartrate, esylate, L-aspartate, besylate, tosylate, sulphate, phosphate, citrate, acetate, L-glutamate, maleate, gentisate, glucuronate, malonate, naphthylene-2-sulfonate and oxalate” are used herein in their conventional sense to denote salts formed from hydrobromic, methanesulphonic, L-tartaric, ethanesulphonic, L-aspartic, benzenesulphonic, p-toluenesulphonic, sulphuric, phosphoric, citric, acetic, L-glutamic, maleic, gentisic, glucuronic, malonic, naphthylene-2-sulphonic, ethane-1,2-disulphonic, naphthalene-1,5-disulphonic and oxalic acids respectively.

It has also been found that a number of salts of 5-[[5-[4-(4-fluoro-1-methyl-4-piperidyl)-2-methoxy-phenyl]-1H-pyrazol-3-yl]amino]pyrazine-2-carbonitrile have improved properties compared to the hydrochloric acid salt disclosed in WO2015/12039.

Accordingly, in another embodiment (Embodiment 1.2), the invention provides a pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile which is selected from maleate, tosylate, besylate and malonate salts.

The compound 5-[[5-[4-(4-fluoro-1-methyl-4-piperidyl)-2-methoxy-phenyl]-1H-pyrazol-3-yl]amino]pyrazine-2-carbonitrile has the formula (1) below, and the salts of the maleate, tosylate, besylate and malonate salts of 5-[[5-[4-(4-fluoro-1-methyl-4-piperidyl)-2-methoxy-phenyl]-1H-pyrazol-3-yl]amino]pyrazine-2-carbonitrile may be referred to herein for convenience as the salts of the compound of formula (1) or the salts of the invention.

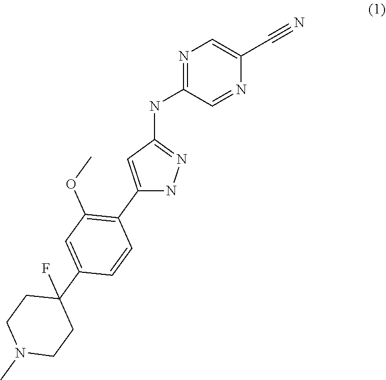 [SMILES: COc1cc(C2(F)CCN(C)CC2)ccc1-c1cc(Nc2cnc(C#N)cn2)n[nH]1]

The compound of formula (1) has several basic nitrogen atoms and can, in principle, form salts with differing salt ratios (i.e. molar ratios of free base:acid). For example, where the acid is monobasic, mono salts (i.e. where there is a 1:1 molar ratio of acid to free base) or bis-salts (where there is an molar ratio of acid to free base of approximately 2:1) can be prepared depending on the number of molar equivalents of acid used in the methods used for the salt formation. Where a dibasic acid (e.g. dicarboxylic acid) is used for salt formation, hemi-salts (where the molar ratio of acid to base in the salt is 0.5:1), mono salts and bis salts can be formed depending on the particular salt formation conditions used.

Accordingly, in further embodiments (Embodiments 1.3 to 1.9), the invention provides:
1.3 A pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile which is a maleate salt.
1.3A A pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile which is a crystalline maleate salt having crystal Pattern B as defined herein.
1.4 A pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile which is a tosylate salt.
1.5 A pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile which is a besylate salt.
1.6 A pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile which is a malonate salt.
1.7 A pharmaceutically acceptable salt according to any one of Embodiments 1.1 to 1.6 having a salt ratio (molar ratio of acid:free base) of approximately 1:1.
1.8 A pharmaceutically acceptable salt according to Embodiment 1.3 or Embodiment 1.6 having a salt ratio (molar ratio of acid:free base) of approximately 0.5:1.
1.9 A pharmaceutically acceptable salt according to Embodiment 1.3 or Embodiment 1.5 having a salt ratio (molar ratio of acid:free base) of approximately 2:1.

Salts of the compound of formula (1) can be amorphous or substantially crystalline.

The term “substantially crystalline” refers to salts which are from 50% to 100% crystalline. Within this range, the salts may be at least 55% crystalline, or at least 60% crystalline, or at least 70% crystalline, or at least 80% crystalline, or at least 90% crystalline, or at least 95% crystalline, or at least 98% crystalline, or at least 99% crystalline, or at least 99.5% crystalline, or at least 99.9% crystalline, for example 100% crystalline.

Certain salts of the invention can exist in several different crystalline forms or polymorphs.

In UK patent application number 2107924.9, filed on 3 Jun. 2021, from which the present application claims priority, certain maleate salt forms were labelled as Pattern A, Pattern A′ and Pattern A″. These forms have been relabeled in this application as Pattern A, Pattern B and Pattern C respectively.

The crystalline forms of the salts of the compound of formula (1) are preferably those having a crystalline purity of at least 90%, more preferably at least 95%; i.e. at least 90% (more preferably at least 95%) of the salt is of a single crystalline form.

The crystalline forms of the salts of the invention may be solvated (e.g. hydrated) or non-solvated (e.g. anhydrous).

The term “anhydrous” as used herein does not exclude the possibility of the presence of some water on or in the crystalline form of the salt. For example, there may be some water present on the surface of the crystalline form of the salt, or minor amounts within the body of the crystalline form of the salt. Typically, an anhydrous form contains fewer than 0.4 molecules of water per molecule of the compound of formula (1), and more preferably contains fewer than 0.1 molecules of water per molecule of the compound of formula (1), for example 0 molecules of water.

Where the crystalline forms are hydrated, they can contain, for example, up to three molecules of water of crystallisation, more usually up to two molecules of water, e.g. one molecule of water or two molecules of water. Non-stoichiometric hydrates may also be formed in which the number of molecules of water present is less than one or is otherwise a non-integer. For example, where there is less than one molecule of water present, there may be for example 0.4, or 0.5, or 0.6, or 0.7, or 0.8, or 0.9 molecules of water present per molecule of compound (1).

Accordingly, in further embodiments of the invention (Embodiments 1.10 to 1.11), there are provided:
1.10 A pharmaceutically acceptable salt according to any one of Embodiments 1.1 to 1.9 which is from 50% to 100% crystalline.
1.11 A pharmaceutically acceptable salt according to Embodiment 1.10 which is:
(b) at least 55% crystalline; or
1.11A A pharmaceutically acceptable salt according to Embodiment 1.11 which is at least 60% crystalline.
1.11B A pharmaceutically acceptable salt according to Embodiment 1.11 which is at least 70% crystalline.
1.11C A pharmaceutically acceptable salt according to Embodiment 1.11 which is at least 80% crystalline.
1.11D A pharmaceutically acceptable salt according to Embodiment 1.11 which is at least 90% crystalline.
1.11E A pharmaceutically acceptable salt according to Embodiment 1.11 which is at least 95% crystalline.
1.11F A pharmaceutically acceptable salt according to Embodiment 1.11 which is at least 98% crystalline.
1.11G A pharmaceutically acceptable salt according to Embodiment 1.11 which is at least 99% crystalline.
1.11H A pharmaceutically acceptable salt according to Embodiment 1.11 which is at least 99.5% crystalline.
1.11I A pharmaceutically acceptable salt according to Embodiment 1.11 which is at least 99.9% crystalline.
1.11J A pharmaceutically acceptable salt according to Embodiment 1.11 which is 100% crystalline.

The crystalline forms can be characterised using a number of techniques including X-ray powder diffraction (XRPD), single crystal X-ray diffraction, differential scanning calorimetry (DSC) and thermogravimetric analysis (TGA). The behaviour of the crystals under conditions of varying humidity can be analysed by gravimetric vapour sorption studies (GVS) such as dynamic vapour sorption (DVS).

The crystalline structure of a compound can be analysed by the solid-state technique of X-ray Powder Diffraction (XRPD). XRPD can be carried out according to conventional methods such as those described herein (see the Examples below) and in “Introduction to X-ray Powder Diffraction”, Ron Jenkins and Robert L. Snyder (John Wiley & Sons, New York, 1996). The presence of defined peaks (as opposed to random background noise) in an XRPD diffractogram indicates that the compound has a degree of crystallinity.

A compound's X-ray powder pattern is characterised by the diffraction angle (2θ) (also referred to herein as °2Th or °2Theta) and interplanar spacing (d) parameters of an X-ray diffraction spectrum. These are related by Bragg's equation, nλ=2d Sin θ, (where n=1; λ=wavelength of the X-ray radiation; d=interplanar spacing; and θ=diffraction angle).

Accordingly, in further embodiments (Embodiments 1.12 to 1.42), the invention provides:
1.12 A pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.1 which has an XRPD spectrum substantially as shown in any one of FIGS. 5 to 25, 27, 29, 31, 33 and 35 (disregarding any XRPD spectra for the free base or amorphous salt forms).
1.13 A pharmaceutically acceptable maleate Pattern B salt of 5-[[5-[4-(4-fluoro-1-methyl-4-piperidyl)-2-methoxy-phenyl]-1H-pyrazol-3-yl]amino]pyrazine-2-carbonitrile according to any one of Embodiments 1.2, 1.3 and 1.3A which has an XRPD spectrum substantially as shown in FIG. 25.
1.14 A pharmaceutically acceptable maleate Pattern B salt of 5-[[5-[4-(4-fluoro-1-methyl-4-piperidyl)-2-methoxy-phenyl]-1H-pyrazol-3-yl]amino]pyrazine-2-carbonitrile according to any one of Embodiments 1.2, 1.3, 1.3A, 1.12 and 1.13 which has an XRPD spectrum characterised by a major °2Th (°2Theta) peak at 26.3±0.2° (e.g. having a relative intensity of 100%).
1.14A A pharmaceutically acceptable maleate Pattern B salt of 5-[[5-[4-(4-fluoro-1-methyl-4-piperidyl)-2-methoxy-phenyl]-1H-pyrazol-3-yl]amino]pyrazine-2-carbonitrile according to any one of Embodiments 1.2, 1.3, 1.3A, 1.12 and 1.13 which has an XRPD spectrum characterised by major °2Th (°2Theta) peaks at 6.9±0.2° and/or 26.4±0.2° and/or 11.8±0.2° and/or 17.9±0.2°.
1.15 A pharmaceutically acceptable maleate Pattern B salt according to Embodiment 1.14 which has an XRPD spectrum characterised by major °2Th peaks at 6.9±0.2°, 26.4±0.2°, 11.8±0.2° and 17.9±0.2°.
1.16 A pharmaceutically acceptable maleate Pattern B salt according to Embodiment 1.14 or Embodiment 1.15 which has an XRPD spectrum characterised by intermediate °2Th peaks at 15.6±0.2° and/or 9.4±0.2° and/or 15.8±0.2° and/or 17.7±0.2° and/or 26.8±0.2°.
1.17 A pharmaceutically acceptable maleate Pattern B salt according to Embodiment 1.16 which has an XRPD spectrum characterised by intermediate °2Th peaks at 15.6±0.2°, 9.4±0.2°, 15.8±0.2°, 17.7±0.2° and 26.8±0.2°
1.18 A pharmaceutically acceptable maleate Pattern A salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.2 which has an XRPD spectrum substantially as shown in FIG. 27.
1.19 A pharmaceutically acceptable maleate Pattern A salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.2 or Embodiment 1.18 which has an XRPD spectrum characterised by major °2Th peaks at 6.6±0.2° and/or 17.3±0.2° and/or 11.1±0.2°.
1.20 A pharmaceutically acceptable maleate Pattern A salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.19 which has an XRPD spectrum characterised by major °2Th peaks at 6.6±0.2°, 17.3±0.2° and 11.1±0.2°.
1.21 A pharmaceutically acceptable maleate Pattern A salt according to Embodiment 1.19 or Embodiment 1.20 which has an XRPD spectrum characterised by intermediate °2Th peaks at 26.5±0.2° and/or 9.2±0.2° and/or 14.3±0.2° and/or 18.5±0.2° and/or 25.9±0.2° and/or 11.5±0.2° and/or 16.9±0.2° and/or 20.5±0.2° and/or 15.6±0.2°.
1.22 A pharmaceutically acceptable maleate Pattern A salt according to Embodiment 1.21 which has an XRPD spectrum characterised by intermediate °2Th peaks at 26.5±0.2°, 9.2±0.2°, 14.3±0.2°, 18.5±0.2°, 25.9±0.2°, 11.5±0.2°, 16.9±0.2°, 20.5±0.2° and 15.6±0.2°.
1.23 A pharmaceutically acceptable maleate Pattern C salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.2 which has an XRPD spectrum substantially as shown in FIG. 29.
1.24 A pharmaceutically acceptable maleate Pattern C salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.2 or Embodiment 1.23 which has an XRPD spectrum characterised by major °2Th peaks at 6.7±0.2° and/or 9.2±0.2° and/or 11.5±0.2° and/or 15.6±0.2° and/or 17.4±0.2° and/or 17.7±0.2° and/or 26.3±0.2°.
1.25 A pharmaceutically acceptable maleate Pattern C salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.24 which has an XRPD spectrum characterised by major °2Th peaks at 6.7 0.2°, 9.2 0.2°, 11.5±0.2°, 15.6±0.2°, 17.4±0.2°, 17.7±0.2° and 26.3±0.2°.
1.26 A pharmaceutically acceptable maleate Pattern C salt according to Embodiment 1.24 or Embodiment 1.25 which has an XRPD spectrum characterised by intermediate °2Th peaks at 18.5±0.2° and/or 14.3±0.2° and/or 21.7±0.2° and/or 11.1±0.2° and/or 27.6±0.2° and/or 17.0±0.2° and/or 25.6±0.2° and/or 16.0±0.2° and/or 22.2±0.2°.
1.27 A pharmaceutically acceptable maleate Pattern C salt according to Embodiment 1.26 which has an XRPD spectrum characterised by intermediate °2Th peaks at 18.5 0.2°, 14.3 0.2°, 21.7 0.2°, 11.1±0.2°, 27.6 0.2°, 17.0 0.2°, 25.6±0.2°, 16.0±0.2° and 22.2±0.2°.
1.28 A pharmaceutically acceptable malonate Pattern B salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.12 which has an XRPD spectrum substantially as shown in FIG. 31.
1.29 A pharmaceutically acceptable malonate Pattern B salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.2 or Embodiment 1.28 which has an XRPD spectrum characterised by major °2Th peaks at 10.6±0.2° and/or 6.5±0.2°.
1.30 A pharmaceutically acceptable malonate Pattern B salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.29 which has an XRPD spectrum characterised by major °2Th peaks at 10.6±0.2° and 6.5±0.2°.
1.31 A pharmaceutically acceptable malonate Pattern B salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.29 or Embodiment 1.30 which has an XRPD spectrum characterised by intermediate °2Th peaks at 16.6±0.2° and/or 18.4±0.2° and/or 14.3±0.2° and/or 25.9±0.2°.
1.32 A pharmaceutically acceptable malonate Pattern B salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.31 which has an XRPD spectrum characterised by intermediate °2Th peaks at 16.6 0.2°, 18.4 0.2°, 14.3±0.2° and 25.9±0.2°.
1.33 A pharmaceutically acceptable tosylate Pattern A salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.2 which has an XRPD spectrum substantially as shown in FIG. 33.
1.34 A pharmaceutically acceptable tosylate Pattern A salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.2 or Embodiment 1.33 which has an XRPD spectrum characterised by major °2Th peaks at 9.1±0.2° and/or 22.2±0.2° and/or 14.9±0.2° and/or 13.8±0.2°.
1.35 A pharmaceutically acceptable tosylate Pattern A salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.34 which has an XRPD spectrum characterised by major °2Th peaks at 9.1±0.2°, 22.2±0.2°, 14.9±0.2° and 13.8±0.2°.
1.36 A pharmaceutically acceptable tosylate Pattern A salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.34 or Embodiment 1.35 which has an XRPD spectrum characterised by intermediate °2Th peaks at 11.7±0.2° and/or 8.8±0.2° and/or 15.7±0.2° and/or 17.9±0.2° and/or 16.5±0.2° and/or 24.8±0.2° and/or 22.6±0.2°.
1.37 A pharmaceutically acceptable tosylate Pattern A salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.36 which has an XRPD spectrum characterised by intermediate °2Th peaks at 11.7±0.2°, 8.8±0.2°, 15.7±0.2°, 17.9±0.2°, 16.5±0.2°, 24.8±0.2° and 22.6±0.2°.
1.38 A pharmaceutically acceptable besylate Pattern C salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.2 which has an XRPD spectrum substantially as shown in FIG. 35.
1.39 A pharmaceutically acceptable besylate Pattern C salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.2 or Embodiment 1.38 which has an XRPD spectrum characterised by major °2Th peaks at 15.5±0.2° and/or 14.7±0.2° and/or 25.4±0.2° and/or 20.9±0.2° and/or 18.1±0.2° and/or 11.2±0.2° and/or 13.3±0.2° and/or 16.1±0.2°.
1.40 A pharmaceutically acceptable besylate Pattern C salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.39 which has an XRPD spectrum characterised by major °2Th peaks at 15.5±0.2°, 14.7±0.2°, 25.4±0.2°, 20.9±0.2°, 18.1±0.2°, 11.2±0.2°, 13.3±0.2° and 16.1±0.2°.
1.41 A pharmaceutically acceptable besylate Pattern C salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.39 or Embodiment 1.40 which has an XRPD spectrum characterised by intermediate °2Th peaks at 24.1±0.2° and/or 9.4±0.2° and/or 26.4±0.2° and/or 16.3±0.2° and/or 19.2±0.2° and/or 27.0±0.2°.
1.42 A pharmaceutically acceptable besylate Pattern C salt of 5-[[5-[4-(4-fluoro-1-methyl-4-piperidyl)-2-methoxy-phenyl]-1H-pyrazol-3-yl]amino]pyrazine-2-carbonitrile according to Embodiment 1.41 which has an XRPD spectrum characterised by intermediate °2Th peaks at 24.1±0.2°, 9.4±0.2°, 26.4±0.2°, 16.3±0.2°, 19.2±0.2° and 27.0±0.2°.

In the above Embodiments, the references to “major °2Th peaks” means those peaks that have a relative intensity (relative to the largest peak) of at least 50% whereas the references to “intermediate peaks” means those peaks that have a relative intensity of between 20% and 50%. Peak positions are given to one decimal place ±0.2° although they have been measured to at least four decimal places. The peak positions are generally listed in descending order of relative intensity.

The salts of the invention may also be characterised by their thermal behaviour and in particular by their DSC and TGA analyses. Thus, in further embodiments, the invention provides:
1.43 A pharmaceutically acceptable maleate Pattern B salt of 5-[[5-[4-(4-fluoro-1-methyl-4-piperidyl)-2-methoxy-phenyl]-1H-pyrazol-3-yl]amino]pyrazine-2-carbonitrile as defined in any one of Embodiments 1.13 to 1.17 which has DSC and TGA characteristics substantially as shown in FIG. 26.
1.44 A pharmaceutically acceptable maleate Pattern A salt of 5-[[5-[4-(4-fluoro-1-methyl-4-piperidyl)-2-methoxy-phenyl]-1H-pyrazol-3-yl]amino]pyrazine-2-carbonitrile as defined in any one of Embodiments 1.18 to 1.22 which has DSC and TGA characteristics substantially as shown in FIG. 28.
1.45 A pharmaceutically acceptable maleate Pattern C salt of 5-[[5-[4-(4-fluoro-1-methyl-4-piperidyl)-2-methoxy-phenyl]-1H-pyrazol-3-yl]amino]pyrazine-2-carbonitrile as defined in any one of Embodiments 1.23 to 1.27 which has DSC and TGA characteristics substantially as shown in FIG. 30.
1.46 A pharmaceutically acceptable malonate Pattern B salt of 5-[[5-[4-(4-fluoro-1-methyl-4-piperidyl)-2-methoxy-phenyl]-1H-pyrazol-3-yl]amino]pyrazine-2-carbonitrile as defined in any one of Embodiments 1.28 to 1.32 which has DSC and TGA characteristics substantially as shown in FIG. 32.
1.47 A pharmaceutically acceptable tosylate Pattern A salt of 5-[[5-[4-(4-fluoro-1-methyl-4-piperidyl)-2-methoxy-phenyl]-1H-pyrazol-3-yl]amino]pyrazine-2-carbonitrile as defined in any one of Embodiments 1.33 to 1.37 which has DSC and TGA characteristics substantially as shown in FIG. 34.
1.48 A pharmaceutically acceptable besylate Pattern C salt of 5-[[5-[4-(4-fluoro-1-methyl-4-piperidyl)-2-methoxy-phenyl]-1H-pyrazol-3-yl]amino]pyrazine-2-carbonitrile as defined in any one of Embodiments 1.38 to 1.42 which has DSC and TGA characteristics substantially as shown in FIG. 36.

Of the various salts of 5-[[5-[4-(4-fluoro-1-methyl-4-piperidyl)-2-methoxy-phenyl]-1H-pyrazol-3-yl]amino]pyrazine-2-carbonitrile defined and described above and elsewhere herein, the maleate salt is a preferred salt.

Advantages of the maleate salt are that it is crystalline with one thermodynamically favoured stable form (Pattern B) and shows a low tendency to polymorphism.

Stability studies carried out over a two-week duration with storage at 25° C./60% RH and 40° C./75% RH indicated the maleate salt remained as a free flowing solid with no evidence of deliquescence or agglomeration and no change in polymorphic form with good chemical stability. Data subsequently gathered over a six month period supported these initial findings.

The maleate salt showed improved solubility over the free base in water and improved solubility in gastric fluid when assessing solubility in biorelevant solvents.

### Isotopes

The salts as defined in any one of Embodiments 1.1 to 1.48 may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the salts contain no radioactive isotopes. Such compounds are preferred for therapeutic use. In another embodiment, however, the salts may contain one or more radioisotopes. Salts containing such radioisotopes may be useful in a diagnostic context.

### Methods for the Preparation of the Salts of the Invention

The pharmaceutically acceptable salts of the invention can be prepared from the free base of the compound 5-[[5-[4-(4-fluoro-1-methyl-4-piperidyl)-2-methoxy-phenyl]-1H-pyrazol-3-yl]amino]pyrazine-2-carbonitrile (the compound of formula (1) by the methods set out in the Examples below. The compound of formula (1) can be prepared by the method described in Example 64, Method L in International patent application WO 2015/20390, as shown in Reaction Scheme 1 below.

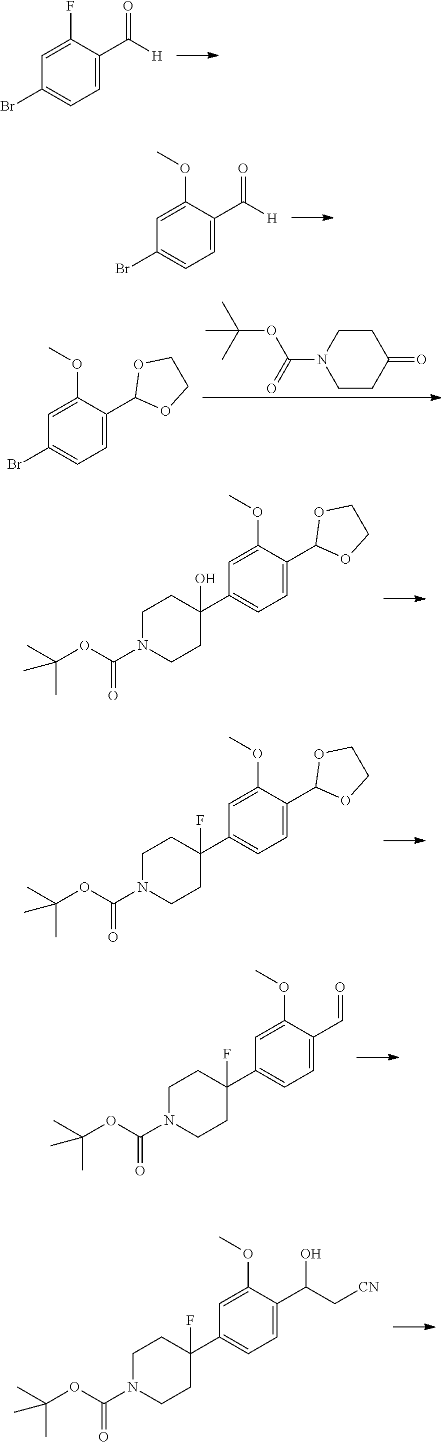

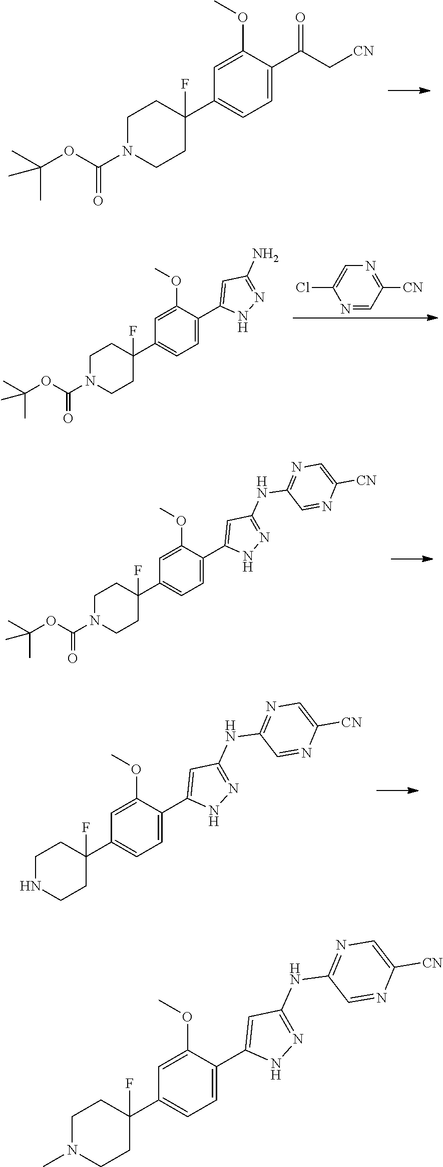 [SMILES: COc1cc(C2(F)CCN(C(=O)OC(C)(C)C)CC2)ccc1C(=O)CC#N ; COc1cc(C2(F)CCN(C(=O)OC(C)(C)C)CC2)ccc1-c1cc(N)n[nH]1 ; N#Cc1cnc(Cl)cn1 ; COc1cc(C2(F)CCN(C(=O)OC(C)(C)C)CC2)ccc1-c1cc(Nc2cnc(C#N)cn2)n[nH]1 ; COc1cc(C2(F)CCNCC2)ccc1-c1cc(Nc2cnc(C#N)cn2)n[nH]1 ; COc1cc(C2(F)CCN(C)CC2)ccc1-c1cc(Nc2cnc(C#N)cn2)n[nH]1]

### Reaction Scheme 1

In further embodiments (Embodiments 2.1 to 2.10), the invention provides methods of forming pharmaceutically acceptable salts of the compound of formula (1), as follows:
2.1 A method of preparing a pharmaceutically acceptable salt as defined in Embodiment 1.1 or Embodiment 1.2; which method comprises dispersing 5-[[5-[4-(4-fluoro-1-methyl-4-piperidyl)-2-methoxy-phenyl]-1H-pyrazol-3-yl]amino]pyrazine-2-carbonitrile in tetrahydrofuran to form a mixture, heating the mixture to an elevated temperature in the range from 45° C. to 65° C. (e.g. from 55° C. to 65° C. and particularly approximately 60° C.), adding a required amount of an acid to the mixture; maintaining the mixture at or near the elevated temperature for a defined period and cooling the mixture to allow isolation of the pharmaceutically acceptable salt.
2.2 A method according to Embodiment 2.1 wherein the acid is selected from maleic acid, p-toluene sulphonic acid, benzene sulphonic acid and malonic acid.
2.3 A method of preparing a pharmaceutically acceptable salt as defined in Embodiment 1.1 or Embodiment 1.2; which method comprises dispersing 5-[[5-[4-(4-fluoro-1-methyl-4-piperidyl)-2-methoxy-phenyl]-1H-pyrazol-3-yl]amino]pyrazine-2-carbonitrile in a mixture of tetrahydrofuran and acetonitrile (e.g. a 1:1 mixture) to form a mixture, heating the mixture to an elevated temperature in the range from 45° C. to 55° C. (e.g. approximately 50° C.), adding a required amount of an acid to the mixture; maintaining the mixture at or near the elevated temperature for a defined period and cooling the mixture to allow isolation of the pharmaceutically acceptable salt.
2.4 A method according to Embodiment 2.3 wherein the acid is selected from maleic acid, p-toluene sulphonic acid and benzene sulphonic acid.
2.5 A method of preparing a pharmaceutically acceptable salt as defined in Embodiment 1.1 or Embodiment 1.2; which method comprises dispersing 5-[[5-[4-(4-fluoro-1-methyl-4-piperidyl)-2-methoxy-phenyl]-1H-pyrazol-3-yl]amino]pyrazine-2-carbonitrile in a mixture of tetrahydrofuran and water (e.g. wherein the mixture contains from 75% to 97% (v/v) tetrahydrofuran and from 3% to 25% (v/v) water, and more preferably approximately 95% (v/v) tetrahydrofuran and approximately 5 (v/v) water) to form a mixture, heating the mixture to an elevated temperature in the range from 45° C. to 65° C. (e.g. approximately 50° C. to 60° C.), adding a required amount of an acid to the mixture; maintaining the mixture at or near the elevated temperature for a defined period and cooling the mixture to allow isolation of the pharmaceutically acceptable salt.
2.6 A method according to Embodiment 2.5 wherein the acid is selected from maleic acid, p-toluene sulphonic acid, benzene sulphonic acid and malonic acid.
2.7 A method according to Embodiment 2.1 wherein the required amount of an acid is an excess of acid (for example up to a 1 molar excess).
2.8 A method according to Embodiment 2.7 wherein the acid is p-toluene sulphonic acid.
2.9 A method according to Embodiment 2.5 wherein the required amount of an acid is an excess of acid (for example up to a 1 molar excess).
2.10 A method according to Embodiment 2.9 wherein the acid is selected from p-toluene sulphonic acid and benzene sulphonic acid.
2.11 A method according to any one of Embodiments 2.1, 2.3 and 2.5 wherein the acid is maleic acid and the resulting pharmaceutically acceptable salt is a maleate salt.
2.12 A method according to Embodiment 2.11 wherein the maleate salt is maleate salt Pattern A salt.
2.13 A method according to Embodiment 2.13 which further comprises converting the Pattern A maleate salt to a Pattern B maleate salt by conditioning the Pattern A salt in an atmosphere of greater than 50% relative humidity (e.g. 51% to 90% relative humidity, or 51% to 85% relative humidity).
2.14 A method according to Embodiment 2.13 wherein Pattern A maleate salt is conditioned in an atmosphere of greater than 60% relative humidity and a temperature in the range from 35-45° C.
2.15 A method according to Embodiment 2.13 or Embodiment 2.14 wherein the Pattern A maleate salt is conditioned in an atmosphere of 70% to 80% relative humidity.
2.16 A method according to Embodiment 2.14 wherein Pattern A maleate salt is conditioned in an atmosphere of approximately 75% relative humidity and a temperature of approximately 40° C.

Particular sets of conditions for performing the above methods are as set out in the Examples below.

### Biological Properties and Therapeutic Uses

The compound of formula (1) and its salts are potent inhibitors of Chk-1 and consequently are expected to be beneficial alone or in combination with various chemotherapeutic agents, immunotherapy agents or radiation for treating a wide spectrum of proliferative disorders.

Accordingly, in further embodiments (Embodiments 3.1 to 3.10), the invention provides:
3.1 A pharmaceutically acceptable salt as defined in any one of Embodiments 1.1 to 1.48 for use in medicine or therapy.
3.2 A pharmaceutically acceptable salt as defined in any one of Embodiments 1.1 to 1.48 for use as a Chk-1 kinase inhibitor.
3.3 A pharmaceutically acceptable salt as defined in any one of Embodiments 1.1 to 1.48 for use in enhancing a therapeutic effect of radiation therapy or chemotherapy or immunotherapy in the treatment of a proliferative disease such as cancer.
3.4 A pharmaceutically acceptable salt as defined in any one of Embodiments 1.1 to 1.48 for use in the treatment of a proliferative disease such as cancer.
3.5 The use of a pharmaceutically acceptable salt as defined in any one of Embodiments 1.1 to 1.48 for the manufacture of a medicament for enhancing a therapeutic effect of radiation therapy or chemotherapy or immunotherapy in the treatment of a proliferative disease such as cancer.
3.6 The use of a pharmaceutically acceptable salt as defined in any one of Embodiments 1.1 to 1.48 for the manufacture of a medicament for the treatment of a proliferative disease such as cancer.
3.7 A method for the prophylaxis or treatment of a proliferative disease such as cancer, which method comprises administering to a patient in combination with radiotherapy, immunotherapy or chemotherapy a pharmaceutically acceptable salt as defined in any one of Embodiments 1.1 to 1.48.
3.8 A method for the prophylaxis or treatment of a proliferative disease such as cancer, which method comprises administering to a patient a pharmaceutically acceptable salt as defined in any one of Embodiments 1.1 to 1.48.
3.9 A pharmaceutically acceptable salt for use, use or method as defined in any one of Embodiments 3.3 to 3.8 wherein the cancer is selected from carcinomas, for example carcinomas of the bladder, brain, breast, colon, kidney, epidermis, liver, lung, oesophagus, gall bladder, ovary, pancreas, stomach, cervix, thyroid, prostate, gastrointestinal system, or skin, hematopoietic tumours such as leukaemia, B-cell lymphoma, T-cell lymphoma, Hodgkin's lymphoma, non-Hodgkin's lymphoma, hairy cell lymphoma, mantle cell lymphoma or Burkett's lymphoma; hematopoietic tumours of myeloid lineage, for example acute and chronic myelogenous leukaemias, myelodysplastic syndrome, or promyelocytic leukaemia; thyroid follicular cancer; tumours of mesenchymal origin, for example fibrosarcoma or rhabdomyosarcoma; tumours of the central or peripheral nervous system, for example astrocytoma, neuroblastoma, glioma, medulloblastoma or schwannoma; melanoma; seminoma; teratocarcinoma; osteosarcoma; xeroderma pigmentosum; keratoctanthoma; thyroid follicular cancer; Ewing's sarcoma or Kaposi's sarcoma.
3.10 A pharmaceutically acceptable salt for use, use or method according to Embodiment 3.9 wherein the cancer is selected from breast cancer, colon cancer, lung cancer, ovarian cancer, pancreatic cancer, prostate cancer, glioma, Ewing's sarcoma, lymphoma (e.g. mantle cell lymphoma), medulloblastoma and leukemia.

It is also envisaged that the pharmaceutically acceptable salts of formula (1) described herein may be useful in treating:
(a) cancers driven by oncogenes including Myc and CCNE1;
(b) cancers with deregulated cell cycle or DNA damage repair pathway, such as those with deficiencies in RAD17 (e.g. RAD17 mutant tumours), RAD50, TP53, or ATM (e.g. tumours in which there is a defective DNA repair mechanism or a defective cell cycle such as a cancer in which mutations (e.g. in p53) have led to the G1/S DNA damage checkpoint being lost), or fanconi anaemia; and
(c) cancers with high levels of replicative stress, such as with amplification of Chk1 or ATR.

Accordingly in further embodiments (Embodiments 3.11 to 3.23), the invention provides:
3.11 A pharmaceutically acceptable salt for use, a use or a method as defined in any one of Embodiments 3.3 to 3.10 wherein the cancer is one which is characterized by a defective DNA repair mechanism or defective cell cycle or high levels of replication stress.
3.12 A pharmaceutically acceptable salt for use, a use or a method according to Embodiment 3.11 wherein the cancer is a p53 negative or mutated tumour.
3.13 A pharmaceutically acceptable salt for use, a use or a method as defined in any one of Embodiments 3.3 to 3.10 wherein the cancer is an MYC oncogene-driven cancer.
3.14 A pharmaceutically acceptable salt for use, a use or a method according to Embodiment 3.13 wherein the MYC oncogene-driven cancer is a B-cell lymphoma, leukaemia, neuroblastoma, medulloblastoma, breast cancer or lung cancer.
3.15 A pharmaceutically acceptable salt as defined in any one of Embodiments 1.1 to 1.48 for use in the treatment of a patient suffering from a p53 negative or mutated tumour (e.g. a cancer selected from breast cancer, colon cancer, lung cancer, ovarian cancer, pancreatic cancer, prostate cancer, glioma, and leukemia) in combination with radiotherapy, immunotherapy or chemotherapy.
3.16 A pharmaceutically acceptable salt for use according to any one of Embodiments 3.3 to 3.15 wherein, in addition to administration of a pharmaceutically acceptable salt as defined in any one of Embodiments 1.1 to 1.48, the treatment comprises administration to a patient of a chemotherapeutic agent selected from cytarabine, etoposide, gemcitabine, cyclophosphamide, a Wee1 inhibitor and SN-38.
3.17 The use of a pharmaceutically acceptable salt as defined in any one of Embodiments 1.1 to 1.48 for the manufacture of a medicament for the treatment of a patient suffering from a cancer which is characterised by a defective DNA repair mechanism or defective cell cycle or high levels of replication stress.
3.18 The use according to Embodiment 3.17 wherein the cancer is a p53 negative or mutated tumour.
3.19 A method for the treatment of a patient (e.g. a human patient) suffering from a cancer which is characterised by a defective DNA repair mechanism or defective cell cycle or high levels of replication stress, which method comprises administering to the patient a therapeutically effective amount of a pharmaceutically acceptable salt as defined in any one of Embodiments 1.1 to 1.48.
3.20 A method according to Embodiment 3.19 wherein the cancer is a p53 negative or mutated tumour.
3.21 A pharmaceutically acceptable salt for use, a use or a method as defined in any one of Embodiments 3.3 to 3.10 wherein the cancer is a RAD17-mutant tumour or an ATM-deficient RAD50-mutant tumour.
3.21 A pharmaceutically acceptable salt as defined in any one of Embodiments 1.1 to 1.48 for use in the treatment of Fanconi anaemia.
3.22 The use of a pharmaceutically acceptable salt as defined in any one of Embodiments 1.1 to 1.48 for the manufacture of a medicament for the treatment of Fanconi anaemia.
3.23 A method of treating Fanconi anaemia in a subject (e.g. a human subject) in need thereof, which method comprises administering to the subject a therapeutically effective amount of a pharmaceutically acceptable salt as defined in any one of Embodiments 1.1 to 1.48.

The Chk-1 inhibitor salts of the invention may be used alone or they may be used in combination with DNA-damaging anti-cancer drugs and/or radiation therapy and/or immunotherapy to treat subjects with multi-drug resistant cancers. A cancer is considered to be resistant to a drug when it resumes a normal rate of tumour growth while undergoing treatment with the drug after the tumour had initially responded to the drug. A tumour is considered to “respond to a drug” when it exhibits a decrease in tumour mass or a decrease in the rate of tumour growth.

Prior to administration of a pharmaceutically acceptable salt as defined in any one of Embodiments 1.1 to 1.48, a patient may be screened to determine whether a cancer from which the patient is or may be suffering is one which would be susceptible to treatment with either a Chk-1 kinase inhibitor compound or a combination of a chemotherapeutic agent (such as a DNA-damaging agent) and a Chk-1 kinase inhibitor compound.

More particularly, a patient may be screened to determine whether a cancer from which the patient is or may be suffering is one which is characterised by a defective DNA repair mechanism or a defective cell cycle or high levels of replication stress, for example a defective cell cycle due to a p53 mutation or is a p53 negative cancer.

Cancers which are characterised by p53 mutations or the absence of p53 can be identified, for example, by the methods described in Allred et al., J. Nat. Cancer Institute, Vol. 85, No. 3, 200-206 (1993) and the methods described in the articles listed in the introductory part of this application. For example, p53 protein may be detected by immuno-histochemical methods such as immuno-staining.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

In addition to p53, mutations to other DNA repair factors such as RAD17, RAD50, and members of the Fanconi's anaemia complementation group may be predictive of response to Chk1 inhibitors alone, or in combination with chemotherapy.

Cancers which contain mutations in these DNA repair pathways may be identified by DNA sequence analysis of tumour biopsy tissue or circulating tumour DNA (ctDNA) or, in the case of Fanconi's anaemia, by evaluating DNA foci formation in tumour biopsy specimens using an antibody to FANCD2, as described in Duan et al., Frontiers in Oncology vol. 4, 1-8 (2014).

Thus, the pharmaceutically acceptable salts as defined in any one of Embodiments 1.1 to 1.48 may be used to treat members of a sub-population of patients who have been screened (for example by testing one or more biological samples taken from the said patients) and have been found to be suffering from a cancer characterised by p53 mutation or a p53 negative cancer, or a cancer containing a RAD17 or RAD50 mutation, or a mutation in a member of the Fanconi's anaemia complementation group.

Accordingly, in further embodiments (Embodiments 3.24 to 3.30), the invention provides:
3.24 A pharmaceutically acceptable salt as defined in any one of Embodiments 1.1 to 1.48 for use in the treatment of a cancer in a subject (e.g. a human subject) who has been screened and has been determined as suffering from a cancer which would be susceptible to treatment with either a Chk-1 kinase inhibitor compound or a combination of a chemotherapeutic agent (such as a DNA-damaging agent) and a Chk-1 kinase inhibitor compound.
3.25 A pharmaceutically acceptable salt as defined in any one of Embodiments 1.1 to 1.48 for use in the treatment of a cancer in a subject (e.g. a human subject) who has been screened and has been determined as suffering from a cancer which is characterised by a defective DNA repair mechanism or a defective cell cycle, for example a defective cell cycle due to a p53 mutation or is a p53 negative cancer.
3.26 A pharmaceutically acceptable salt as defined in any one of Embodiments 1.1 to 1.48 for use in the treatment of a cancer in a subject (e.g. a human subject) who has been screened and has been determined as suffering from a cancer characterised by p53 mutation or a p53 negative cancer, or a cancer containing a RAD17 or RAD50 mutation, or a mutation in a member of the Fanconi's anaemia complementation group.
3.27 The use of a pharmaceutically acceptable salt as defined in any one of Embodiments 1.1 to 1.48 for the manufacture of a medicament for a use as defined in any one of Embodiments 3.24 to 3.26.
3.28 A method for the treatment of a cancer in a subject (e.g. a human subject) who has been screened and has been determined as suffering from a cancer which would be susceptible to treatment with either a Chk-1 kinase inhibitor compound or a combination of a chemotherapeutic agent (such as a DNA-damaging agent) and a Chk-1 kinase inhibitor compound, which method comprises the administration of a therapeutically effective amount of pharmaceutically acceptable salt as defined in any one of Embodiments 1.1 to 1.48 and optionally a chemotherapeutic agent (such as a DNA-damaging agent).
3.29 A method for the treatment of a cancer in a subject (e.g. a human subject) who has been screened and has been determined as suffering from a cancer which is characterised by a defective DNA repair mechanism or a defective cell cycle, for example a defective cell cycle due to a p53 mutation or is a p53 negative cancer, which method comprises the administration of a therapeutically effective amount of a pharmaceutically acceptable salt as defined in any one of Embodiments 1.1 to 1.48.
3.30 A method for the treatment of a cancer in a subject (e.g. a human subject) who has been screened and has been determined as suffering from a cancer characterised by p53 mutation or a p53 negative cancer, or a cancer containing a RAD17 or RAD50 mutation, or a mutation in a member of the Fanconi's anaemia complementation group, which method comprises administering to the subject a therapeutically effective amount of a pharmaceutically acceptable salt as defined in any one of Embodiments 1.1 to 1.48.

### Combination Therapy

It is envisaged that the pharmaceutically acceptable salts as defined in any one of Embodiments 1.1 to 1.48 will be useful either alone or in combination therapy with chemotherapeutic agents (particularly DNA-damaging agents) or radiation therapy or immunotherapy in the prophylaxis or treatment of a range of proliferative disease states or conditions. Examples of such disease states and conditions are set out above.

The pharmaceutically acceptable salts as defined in any one of Embodiments 1.1 to 1.48, whether administered alone, or in combination with DNA damaging agents and other anti-cancer agents and therapies, are generally administered to a subject in need of such administration, for example a human or animal patient, preferably a human.

According to another embodiment of the invention, Embodiment 4.1, there is provided a combination of a pharmaceutically acceptable salt as defined in any one of Embodiments 1.1 to 1.48 together with another chemotherapeutic agent, for example an anticancer drug.

Examples of chemotherapeutic agents that may be co-administered with the pharmaceutically acceptable salts as defined in any one of Embodiments 1.1 to 1.48 include:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Hypoxia triggered DNA damaging agents (e.g. Tirapazamine, TH-302)

Particular examples of chemotherapeutic agents that may be administered in combination with the pharmaceutically acceptable salts as defined in any one of Embodiments 1.1 to 1.48 include:
nitrogen mustards such as mechlorethamine, cyclophosphamide, ifosfamide, melphalan and chlorambucil;
nitrosoureas such as carmustine, lomustine and semustine;
ethyleneimine/methylmelamine compounds such as triethylenemelamine, triethylene thiophosphoramide and hexamethylmelamine;
alkyl sulphonates such as busulfan;
triazines such as dacarbazine;
Antimetabolites such as folates, methotrexate, trimetrexate, 5-fluorouracil, fluorodeoxyuridine, gemcitabine, cytosine arabinoside, 5-azacytidine, 2, 2′-difluorodeoxycytidine, 6-mercaptopurine, 6-thioguanine, azathioprine, 2′-deoxycoformycin, erythrohydroxynonyl-adenine, fludarabine phosphate and 2-chlorodeoxyadenosine;
type I topoisomerase inhibitors such as camptothecin, topotecan and irinotecan;
type II topoisomerase inhibitors such as the epipodophylotoxins (e.g. etoposide and teniposide);
antimitotic drugs such as paclitaxel, Taxotere, Vinca alkaloids (e.g. vinblastine, vincristine, vinorelbine) and estramustine (e.g. estramustine phosphate);
antibiotics such as actimomycin D, daunomycin (rubidomycin), doxorubicin (adriamycin), mitoxantrone, idarubicine, bleomycin, mithramycin, mitomycin C and dactinomycin enzymes such as L-asparaginase;
cytokines and biological response modifiers such as interferon (α, β, γ), interleukin-2G-CSF and GM-CSF:
retinoids such as retinoic acid derivatives (e.g. bexarotene);
radiosensitisers such as metronidazole, misonidazole, desmethylmisonidazole, pimonidazole, etanidazole, nimorazole, nicotinamide, 5-bromodeoxyuridine, 5-iododeoxyuridine and bromodeoxycytidine;
platinum compounds such as cisplatin, carboplatin, spiroplatin, iproplatin, onnaplatin, tetraplatin and oxaliplatin;
anthracenediones such as mitoxantrone;
ureas such as hydroxyurea;
hydrazine derivatives such as N-methylhydrazine and procarbazine; adrenocortical suppressants such as mitotane and aminoglutethimide; adrenocorticosteroids and antagonists such as prednisone, dexamethasone and aminoglutethimide;
progestins such as hydroxyprogesterone (e.g. hydroxyprogesterone caproate), medroxyprogesterone (e.g. medroxyprogesterone acetate) and megestrol (e.g. megestrol acetate);
oestrogens such as diethylstilbestrol and ethynyl estradiol; anti-oestrogens such as tamoxifen;
androgens such as testosterone (e.g. testosterone propionate) and fluoxymesterone;
anti-androgens such as flutamide and leuprolide;
nonsteroidal anti-androgens such as flutamide; and
signal transduction inhibitors such as PARP inhibitors [e.g. as disclosed in Cancer Res.; 66: (16)], Mek inhibitors [e.g as disclosed in Blood. 2008; 112(6): 2439-2449], farnesyltransferase inhibitors [e.g. as disclosed in Blood. 2005 Feb. 15; 105(4):1706-16], wee1 inhibitors [e.g.as disclosed in Haematologica 2014, 99(4):68], rapamycin and Src inhibitors [e.g as disclosed in Blood. 2011 Feb. 10; 117(6):1947-57].
immunotherapy agents such as anti-PD-L1 [e.g. as disclosed in Cancer Discov. 2019 (5):646]

Examples of the chemotherapeutic agents than may be used in combination with the pharmaceutically acceptable salts as defined in any one of Embodiments 1.1 to 1.48 include the chemotherapeutic agents described in Blasina et al., Mol. Cancer Ther., 2008, 7(8), 2394-2404, Ashwell et al., Clin. Cancer Res., 2008, 14(13), 4032-4037, Ashwell et al., Expert Opin. Investig. Drugs, 2008, 17(9), 1331-1340, Trends in Molecular Medicine February 2011, Vol. 17, No. 2 and Clin Cancer Res; 16(2) Jan. 15, 2010.

Particular examples of chemotherapeutic agents that may be used in combination with the pharmaceutically acceptable salts as defined in any one of Embodiments 1.1 to 1.48 include antimetabolites (such as capecitabine, cytarabine, fludarabine, gemcitabine and pemetrexed), Topoisomerase-I inhibitors (such as SN38, topotecan, irinotecan), platinum compounds (such as carboplatin, oxaloplatin and cisplatin), Topoisomerase-II inhibitors (such as daunorubicin, doxorubicin and etoposide), thymidylate synthase inhibitors (such as 5-fluoruracil), mitotic inhibitors (such as docetaxel, paclitaxel, vincristine and vinorelbine) and alkylating agents (such as mitomycin C).

A further set of chemotherapeutic agents that may be used in combination with the pharmaceutically acceptable salts as defined in any one of Embodiments 1.1 to 1.48 includes agents that induce stalled replication forks (see Ashwell et al., Clin. Cancer Res., above), and examples of such compounds include gemcitabine, 5-fluorouracil and hydroxyurea.

### Posology

The pharmaceutically acceptable salt as defined in any one of Embodiments 1.1 to 1.48 or the therapeutic combinations as defined in Embodiments 4.1 will be administered to a patient in need thereof (for example a human or animal patient) in an amount sufficient to achieve the desired therapeutic effect: e.g. an effect as set out in Embodiments 3.1 to 3.30 above.

The pharmaceutically acceptable salt as defined in any one of Embodiments 1.1 to 1.48 or the therapeutic combinations as defined in Embodiments 4.1 will generally be administered to a subject in need of such administration, for example a human or animal patient, preferably a human.

The pharmaceutically acceptable salt as defined in any one of Embodiments 1.1 to 1.48 or the therapeutic combinations as defined in Embodiments 4.1 will typically be administered in amounts that are therapeutically or prophylactically useful and which generally are non-toxic. However, in certain situations, the benefits of administering the pharmaceutically acceptable salts of the invention or the therapeutic combinations as defined in Embodiment 4.1 may outweigh the disadvantages of any toxic effects or side effects, in which case it may be considered desirable to administer administering the pharmaceutically acceptable salt of the invention or the therapeutic combinations as defined in Embodiment 4.1 in amounts that are associated with a degree of toxicity.
The pharmaceutically acceptable salt as defined in any one of Embodiments 1.1 to 1.48 and combinations with chemotherapeutic agents or radiation therapies as described and defined above (e.g. as in Embodiment 4.1) may be administered over a prolonged term to maintain beneficial therapeutic effects or may be administered for a short period only. Alternatively, they may be administered in a pulsatile or continuous manner.

The pharmaceutically acceptable salt as defined in any one of Embodiments 1.1 to 1.48 or the therapeutic combinations as defined in Embodiment 4.1 will be administered in an effective amount, i.e. an amount which is effective to bring about the desired therapeutic effect either alone (in monotherapy) or in combination with one or more chemotherapeutic agents or radiation therapy. For example, the “effective amount” can be a quantity of pharmaceutically acceptable salt which, when administered alone or together with a DNA-damaging drug or other anti-cancer drug to a subject suffering from cancer, slows tumour growth, ameliorates the symptoms of the disease and/or increases longevity. More particularly, when used in combination with radiation therapy, with a DNA-damaging drug or other anti-cancer drug, an effective amount of the pharmaceutically acceptable salt of the invention is the quantity in which a greater response is achieved when the pharmaceutically acceptable salt is co-administered with the DNA damaging anti-cancer drug and/or radiation therapy compared with when the DNA damaging anti-cancer drug and/or radiation therapy is administered alone. When used as a combination therapy, an “effective amount” of the DNA damaging drug and/or an “effective” radiation dose are administered to the subject, which is a quantity in which anti-cancer effects are normally achieved. The pharmaceutically acceptable salt as defined in any one of Embodiments 1.1 to 1.48 and the DNA damaging anti-cancer drug can be co-administered to the subject as part of the same pharmaceutical composition or, alternatively, as separate pharmaceutical compositions.

When administered as separate pharmaceutical compositions, the pharmaceutically acceptable salt as defined in any one of Embodiments 1.1 to 1.48 and the DNA-damaging anti-cancer drug (and/or radiation therapy) can be administered simultaneously or at different times, provided that the enhancing effect of the pharmaceutically acceptable salt as defined in any one of Embodiments 1.1 to 1.48 is retained.

In one embodiment, a pharmaceutically acceptable salt as defined in any one of Embodiments 1.1 to 1.48 is administered before (e.g. by up to 8 hours or up to 12 hours or up to one day before) administration of the DNA-damaging anticancer drug.

In another embodiment, a pharmaceutically acceptable salt as defined in any one of Embodiments 1.1 to 1.48 is administered after (e.g. by up to 8 hours or up to 12 hours or up to 24 hours or up to 30 hours or up to 48 hours after) administration of the DNA-damaging anticancer drug. In another embodiment, a first dose of a pharmaceutically acceptable salt as defined in any one of Embodiments 1.1 to 1.48 is administered one day after administration of the DNA-damaging anticancer drug and a second dose of the said compound is administered two days after administration of the DNA-damaging anticancer drug.

In a further embodiment, a first dose of a pharmaceutically acceptable salt as defined in any one of Embodiments 1.1 to 1.48 is administered one day after administration of the DNA-damaging anticancer drug, a second dose of the said salt is administered two days after administration of the DNA-damaging anticancer drug, and third dose of the said salt is administered three days after administration of the DNA-damaging anticancer drug.

Particular dosage regimes comprising the administration of a pharmaceutically acceptable salt as defined in any one of Embodiments 1.1 to 1.48 and a DNA-damaging anticancer drug may be as set out in WO2010/118390 (Array Biopharma), the contents of which are incorporated herein by reference.

The amount of pharmaceutically acceptable salt of the invention and (in the case of combination therapy) the DNA damaging anti-cancer drug and radiation dose administered to the subject will depend on the nature and potency of the DNA damaging anti-cancer drug, the type and severity of the disease or condition and on the characteristics of the subject, such as general health, age, sex, body weight and tolerance to drugs. The skilled person will be able to determine appropriate dosages depending on these and other factors. Effective dosages for commonly used anti-cancer drugs and radiation therapy are well known to the skilled person.

A typical daily dose of the pharmaceutically acceptable salt as defined in any one of Embodiments 1.1 to 1.48, whether administered on its own in monotherapy or administered in combination with a DNA damaging anticancer drug, can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound can be administered on a daily basis or on a repeat basis every 2, or 3, or 4, or 5, or 6, or 7, or 10 or 14, or 21, or 28 days for example.

Ultimately, however, the quantity of pharmaceutically acceptable salt administered and the type of composition used will be commensurate with the nature of the disease or physiological condition being treated and will be at the discretion of the physician.

### Pharmaceutical Formulations

The pharmaceutically acceptable salts as defined in any one of Embodiments 1.1 to 1.48 and the therapeutic combinations as defined in Embodiments 4.1 are typically administered to patients in the form of a pharmaceutical composition. Accordingly, in another Embodiment of the invention (Embodiment 5.1), the invention provides a pharmaceutical composition comprising a pharmaceutically acceptable salt as defined in any one of Embodiments 1.1 to 1.48 and a pharmaceutically acceptable excipient, and optionally a further chemotherapeutic agent.

In further embodiments, there are provided:
5.2 A pharmaceutical composition according to Embodiment 5.1 which comprises from approximately 1% (w/w) to approximately 95% (w/w) of a pharmaceutically acceptable salt as defined in any one of Embodiments 1.1 to 1.48 and from 99% (w/w) to 5% (w/w) of a pharmaceutically acceptable excipient or combination of excipients and optionally one or more further therapeutically active ingredients.
5.3 A pharmaceutical composition according to Embodiment 5.2 which comprises from approximately 5% (w/w) to approximately 90% (w/w) of a composition of a pharmaceutically acceptable salt as defined in any one of Embodiments 1.1 to 1.48 and from 95% (w/w) to 10% of a pharmaceutically excipient or combination of excipients and optionally one or more further therapeutically active ingredients.
5.4 A pharmaceutical composition according to Embodiment 5.3 which comprises from approximately 10% (w/w) to approximately 90% (w/w) of a pharmaceutically acceptable salt as defined in any one of Embodiments 1.1 to 1.48 and from 90% (w/w) to 10% of a pharmaceutically excipient or combination of excipients.
5.5 A pharmaceutical composition according to Embodiment 5.4 which comprises from approximately 20% (w/w) to approximately 90% (w/w) of a pharmaceutically acceptable salt as defined in any one of Embodiments 1.1 to 1.48 and from 80% (w/w) to 10% of a pharmaceutically excipient or combination of excipients.
5.6 A pharmaceutical composition according to Embodiment 5.5 which comprises from approximately 25% (w/w) to approximately 80% (w/w) of a pharmaceutically acceptable salt as defined in any one of Embodiments 1.1 to 1.48 and from 75% (w/w) to 20% of a pharmaceutically excipient or combination of excipients.

The pharmaceutical compositions of the invention can be in any form suitable for oral, parenteral, topical, intranasal, intrabronchial, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical dosage forms suitable for oral administration include tablets, capsules, caplets, pills, lozenges, syrups, solutions, sprays, powders, granules, elixirs and suspensions, sublingual tablets, sprays, wafers or patches and buccal patches.

Accordingly, in further embodiments, the invention provides:
5.7 A pharmaceutical composition according to any one of Embodiments 5.1 to 5.6 which is suitable for oral administration.
5.8 A pharmaceutical composition according to Embodiment 5.7 which is selected from tablets, capsules, caplets, pills, lozenges, syrups, solutions, sprays, powders, granules, elixirs and suspensions, sublingual tablets, sprays, wafers or patches and buccal patches.
5.9 A pharmaceutical composition according to Embodiment 5.8 which is selected from tablets and capsules.
5.10 A pharmaceutical composition according to any one of Embodiments 5.1 to 5.6 which is suitable for parenteral administration.
5.11 A pharmaceutical composition according to Embodiment 5.10 which is formulated for intravenous, intramuscular, intraperitoneal, subcutaneous administration or for direct delivery into a target organ or tissue by injection, infusion or other means of delivery.
5.12 A pharmaceutical composition according to Embodiment 5.11 which is a solution or suspension for injection or infusion.

Pharmaceutical compositions (e.g. as defined in any one of Embodiments 5.1 to 5.12) containing a pharmaceutically acceptable salt as defined in any one of Embodiments 1.1 to 1.48 can be formulated in accordance with known techniques, see for example, Remington's Pharmaceutical Sciences, Mack Publishing Company, Easton, PA, USA.

Thus, tablet compositions (as in Embodiment 5.9) can contain a unit dosage of the pharmaceutically acceptable salt as defined in any one of Embodiments 1.1 to 1.48 together with an inert diluent or carrier such as a sugar or sugar alcohol, e.g.; lactose, sucrose, sorbitol or mannitol; and/or a non-sugar derived diluent such as sodium carbonate, calcium phosphate, talc, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations (as in Embodiment 5.9) may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the pharmaceutically acceptable salt at a desired location within the gastro-intestinal tract. Thus, the coating can be selected so as to degrade under certain pH conditions within the gastrointestinal tract, thereby selectively release the pharmaceutically acceptable salt in the stomach or in the ileum or duodenum.

Instead of, or in addition to, a coating, the drug can be presented in a solid matrix comprising a release controlling agent, for example a release delaying agent which may be adapted to selectively release the pharmaceutically acceptable salt under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral administration (as in Embodiments 5.10 to 5.12) are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped mouldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the pharmaceutically acceptable salt together with an inert solid powdered diluent such as lactose.

The pharmaceutical compositions will generally be presented in unit dosage form and, as such, will typically contain sufficient pharmaceutically acceptable salt to provide a desired level of biological activity. For example, a pharmaceutical composition according to any one of Embodiments 5.1 to 5.9), a composition intended for oral administration may contain from 2 milligrams to 200 milligrams of the pharmaceutically acceptable salt, more usually from 10 milligrams to 100 milligrams, for example, 12.5 milligrams, 25 milligrams and 50 milligrams.

The pharmaceutical compositions may optionally include a further chemotherapeutic agent as defined in Embodiment 4.1.

Accordingly, in a further embodiment (Embodiment 5.13), the invention provides a pharmaceutical composition as defined in any one of Embodiments 5.2 to 5.12 which additionally comprises a further chemotherapeutic agent as defined in Embodiment 4.1.

### BRIEF DESCRIPTION OF THE DRAWINGS

FIG. 1 is an XRPD spectrum for the free base of 5-[[5-[4-(4-fluoro-1-methyl-4-piperidyl)-2-methoxy-phenyl]-1H-pyrazol-3-yl]amino]pyrazine-2-carbonitrile (“the compound of formula (1)”).

FIG. 2 shows DSC and TGA traces for the free base of the compound of formula (1).

FIG. 3 shows the GVS profile of the free base of the compound of formula (1).

FIG. 4 shows the XRPD spectra for the free base (top trace) and several crystalline forms of the hydrochloric acid salt of the compound of formula (1). From the second trace from the top down to the bottom trace, the crystalline forms of the salt in order are Patterns A, B, C, D and E.

FIG. 5 shows the XRPD spectra for the free base (top trace) and several crystalline forms of the hydrobromic acid salt of the compound of formula (1). From the second trace from the top down to the bottom trace, the crystalline forms of the salt in order are Patterns A, B, C and D.

FIG. 6 shows the XRPD spectra for the free base (top trace) and several crystalline forms of the mesylate salt of the compound of formula (1). From the second trace from the top down to the bottom trace, the crystalline forms of the salt in order are Patterns A, B and C.

FIG. 7 shows the XRPD spectra for the free base (top trace) and several forms of the L-tartrate salt of the compound of formula (1). From the second trace from the top down to the bottom trace, the forms of the salt in order are the amorphous form (second trace down), Pattern A (third trace down) and Pattern B (bottom trace).

FIG. 8 shows the XRPD spectra for the free base (top trace) and several crystalline forms of the esylate salt of the compound of formula (1). From the second trace from the top down to the bottom trace, the crystalline forms of the salt in order are Patterns A and B (third and fourth traces down).

FIG. 9 shows the XRPD spectra for the free base (top trace) and a crystalline form (bottom trace) of the L-aspartate salt of the compound of formula (1).

FIG. 10 shows the XRPD spectra for several crystalline forms of the besylate salt of the compound of formula (1). From top to bottom, the crystalline forms of the salt are Patterns A, B and C.

FIG. 11 shows the XRPD spectra for several crystalline forms of the tosylate salt of the compound of formula (1). From top to bottom, the crystalline forms are Patterns A, B, C and D.

FIG. 12 shows the XRPD spectra for the free base and several crystalline forms of the sulphate salt of the compound of formula (1). From top to bottom, the crystalline forms are the free base (top trace) and Patterns A (second and third traces down) and B (bottom trace) of the salt.

FIG. 13 shows the XRPD spectra for the free base and several crystalline forms of the phosphate salt of the compound of formula (1). From top to bottom, the crystalline forms are the free base (top trace), and Patterns A (second and third traces down) and B (bottom trace) of the salt.

FIG. 14 shows the XRPD spectra for the free base and several amorphous and crystalline forms of the citrate salt of the compound of formula (1). From top to bottom, the traces are the free base (top trace), the amorphous salt (second trace down), and Pattern A salt and Pattern B salt.

FIG. 15 shows the XRPD spectra for the free base and several crystalline forms of the acetate salt of the compound of formula (1). From top to bottom, the traces are the free base (top trace), salt Pattern A and salt Pattern B.

FIG. 16 shows the XRPD spectra for the free base (top trace) and the Pattern A crystalline form (bottom trace) of the L-glutamate salt of the compound of formula (1).

FIG. 17 shows the XRPD spectra for several crystalline forms of the maleate salt of the compound of formula (1). From top to bottom, the traces are for Pattern A, Pattern B and Pattern C.

FIG. 18 shows the XRPD spectra for the free base (top trace) and the Pattern A crystalline form (middle and bottom traces) of the gentisate salt of the compound of formula (1).

FIG. 19 shows the XRPD spectra for the free base (top trace) and several crystalline forms (Pattern A—middle trace and Pattern B—bottom trace) of the glucuronate salt of the compound of formula (1).

FIG. 20 shows the XRPD spectra for the free base (top trace) and several crystalline forms (Pattern A—middle trace and Pattern B—bottom trace) of the malonate salt of the compound of formula (1).

FIG. 21 shows the XRPD spectra for a crystalline form of the naphthalene-2-sulphonate salt of the compound of formula (1) isolated from THF (top trace) and THF:H₂O (bottom trace).

FIG. 22 shows the XRPD spectra for the free base (top trace) and several crystalline forms (Pattern A—middle trace) and Pattern B (bottom trace) of the oxalate salt of the compound of formula (1).

FIG. 23 shows the XRPD spectra for the free base (top trace) and crystalline forms A, B, C and D (in descending order from the second from top) of the sulphate salt of the compound of formula (1).

FIG. 24 shows the XRPD spectra for the free base (top trace) and crystalline forms D and E (middle and bottom traces) of the sulphate salt of the compound of formula (1).

FIG. 25 shows the XRPD spectrum for the maleate Pattern B salt.

FIG. 26 shows the DSC and TGA traces for the maleate Pattern B salt.

FIG. 27 shows the XRPD spectrum for the maleate Pattern A salt.

FIG. 28 shows the DSC and TGA traces for the maleate Pattern A salt.

FIG. 29 shows the XRPD spectrum for the maleate Pattern C salt.

FIG. 30 shows the DSC and TGA traces for the maleate Pattern C salt.

FIG. 31 shows the XRPD spectrum for the malonate Pattern B salt.

FIG. 32 shows the DSC and TGA traces for the malonate Pattern B salt.

FIG. 33 shows the XRPD spectrum for the tosylate Pattern A salt.

FIG. 34 shows the DSC and TGA traces for the tosylate Pattern A salt.

FIG. 35 shows the XRPD spectrum for the besylate Pattern C salt.

FIG. 36 shows the DSC and TGA traces for the besylate Pattern C salt of the compound of formula (1).

FIG. 37 shows the XRPD spectra for the free base (top trace) and crystalline forms Pattern A (middle trace) and Pattern B (bottom trace) of the bis-mesylate salt of the compound of formula (1).

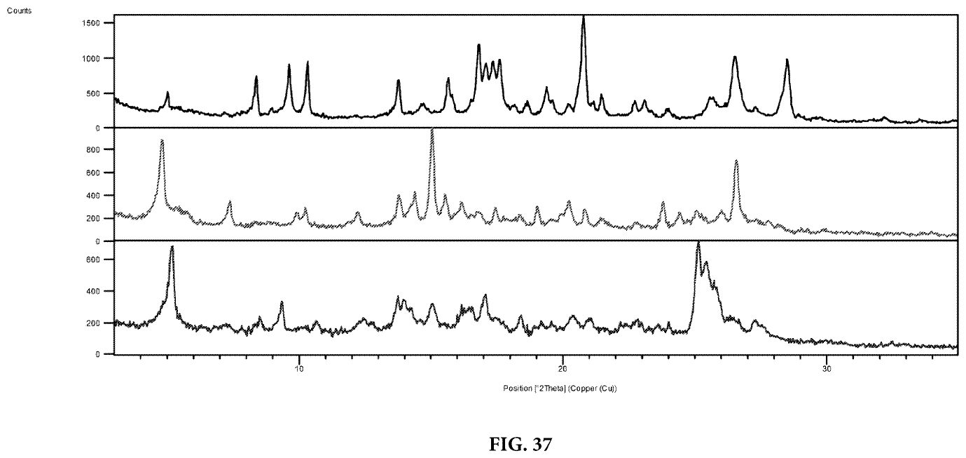

FIG. 38 shows the XRPD spectra for the free base (top trace) and crystalline forms Pattern A (middle trace) and Pattern B (bottom trace) of the bis-maleate salt of the compound of formula (1).

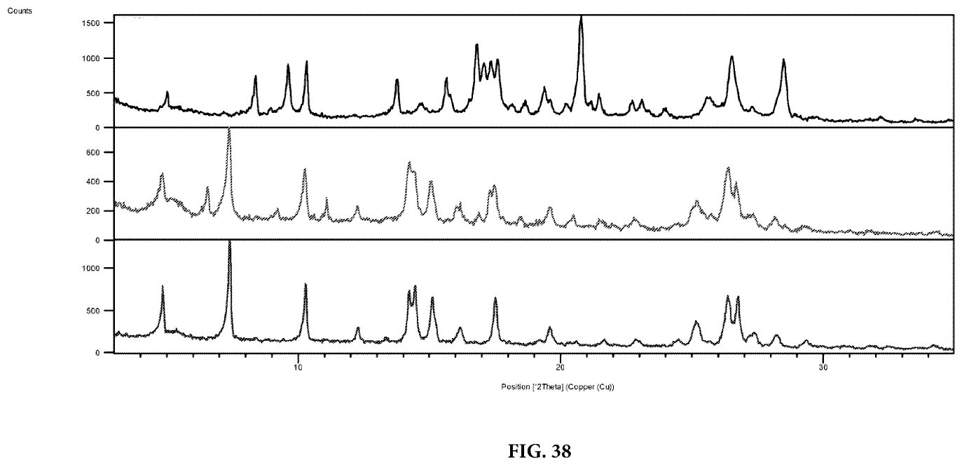

FIG. 39 shows the XRPD spectra for the free base (top trace) and crystalline forms Pattern A (middle trace) and Pattern B (bottom trace) of the bis-besylate salt of the compound of formula (1).

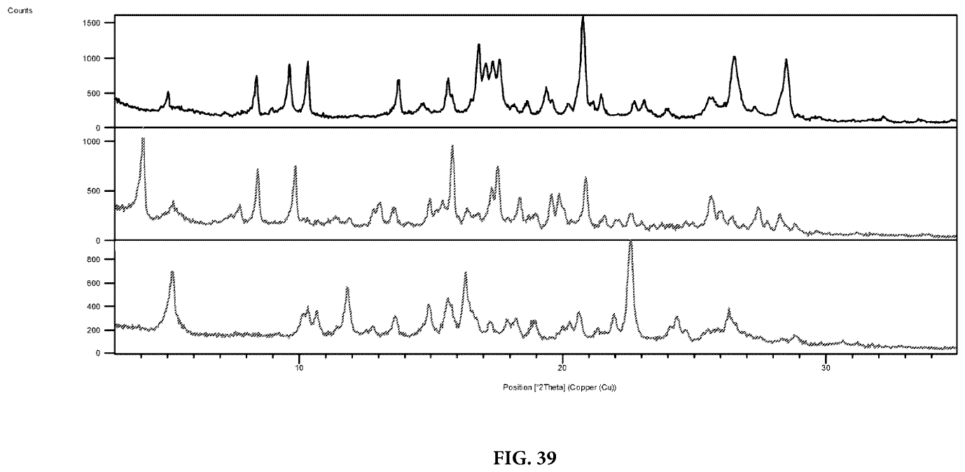

FIG. 40 shows the XRPD spectra for the free base and various crystalline forms of maleate salts. From the top trace to the bottom trace in descending order are the free base, the Pattern A mono-maleate, the Pattern A bis-maleate, the Pattern B bis-maleate and the Pattern A hemi-maleate of the compound of formula (1).

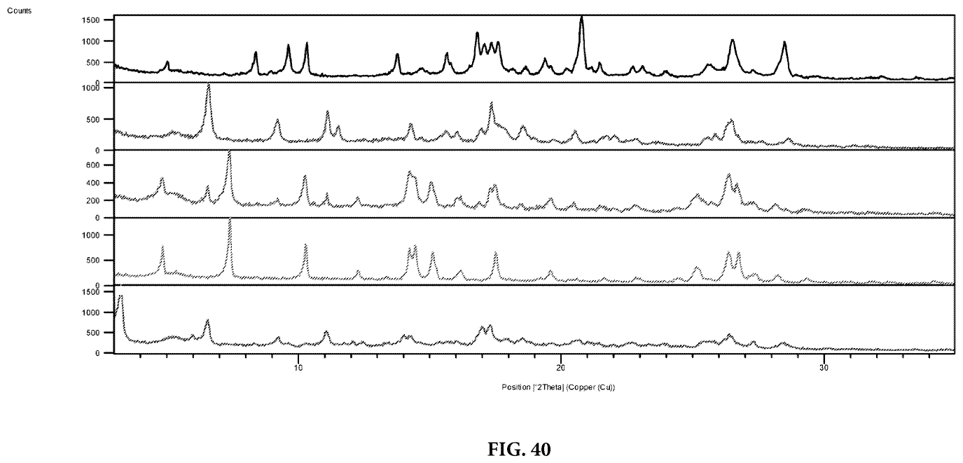

FIG. 41 shows the XRPD spectra for the free base (top trace) and hemi-ethane-1,2-disulphonate salt crystalline form Pattern A (bottom trace).

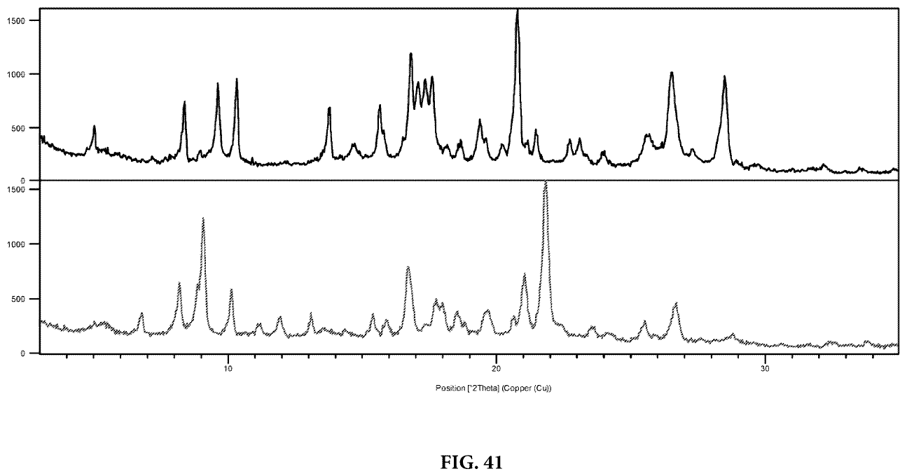

FIG. 42 shows the XRPD spectra for the free base (top trace) and hemi-naphthalene-1,5-disulphonate salt crystalline form Pattern A (bottom trace).

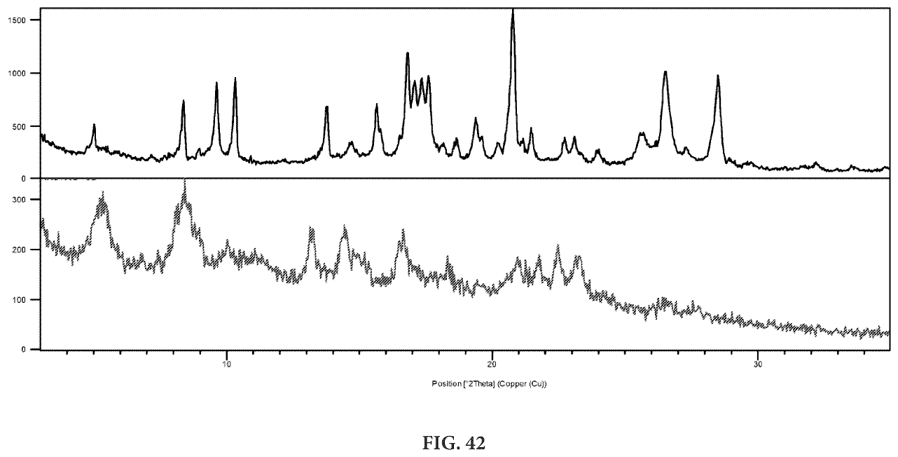

FIG. 43 shows the XRPD spectra for the free base and various crystalline forms of hemi-fumarate salts. From the top trace to the bottom trace in descending order are the free base, the Pattern A hemi-fumarate salt, the Pattern B hemi-fumarate salt and the Pattern C hemi-fumarate salt of the compound of formula (1).

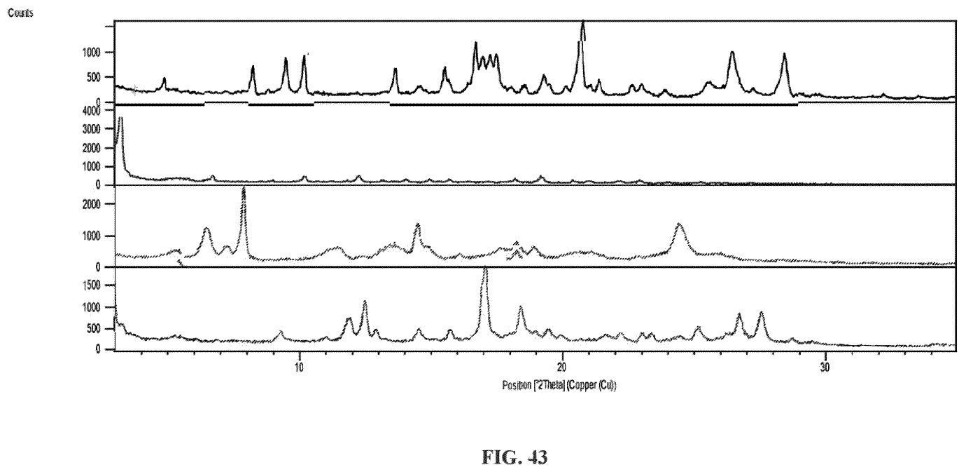

FIG. 44 shows the Gravimetric Vapour Sorption (GVS) plot for the Pattern A crystalline form of the maleate salt of the compound of formula (1).

FIG. 45 shows the GVS plot for the Pattern B crystalline form of the maleate salt of the compound of formula (1).

FIG. 46 shows the GVS plot for the Pattern A crystalline form of the tosylate salt of the compound of formula (1).

FIG. 47 shows the GVS plot for the Pattern A crystalline form of the besylate salt of the compound of formula (1).

FIG. 48 shows the GVS plot for the Pattern B crystalline form of the besylate salt of the compound of formula (1).

FIG. 49 shows the GVS plot for the Pattern C crystalline form of the besylate salt of the compound of formula (1).

FIG. 50 shows the GVS plot for the Pattern A crystalline form of the naphthalene-2-sulphonate salt of the compound of formula (1).

FIG. 51 shows the GVS plot for the Pattern B crystalline form of the malonate salt of the compound of formula (1).

FIG. 52 shows the XRPD patterns for various crystalline forms of the maleate salt. From top to bottom, the crystalline forms are Pattern A, Pattern B, mixture of A/B, Pattern C, Pattern D and Pattern E.

FIG. 53 is a DVS plot for the Pattern B crystalline form of the maleate salt.

FIG. 54 shows the DSC and TGA traces for the maleate Pattern D salt.

FIG. 55 shows the DSC and TGA traces for the maleate Pattern E salt.

### EXAMPLES

### Analytical Methods

### Proton-NMR

Salt formation (by observation of proton shifts vs free base) and identification of the salts as 1:1 (molar ratio of free base:acid) stoichiometric salts were confirmed from their ¹H NMR spectra which were collected using a JEOL ECX 400 MHz spectrometer equipped with an auto-sampler. The samples were dissolved in a suitable deuterated solvent for analysis. The data was acquired using Delta NMR Processing and Control Software version 4.3.

### X-Ray Powder Diffraction (XRPD)

X-Ray Powder Diffraction patterns were collected on a PANalytical diffractometer using Cu Kα radiation (45 kV, 40 mA), θ-θ goniometer, focusing mirror, divergence slit (½″), soller slits at both incident and divergent beam (4 mm) and a PIXcel detector. The software used for data collection was X'Pert Data Collector, version 2.2f and the data was presented using X'Pert Data Viewer, version 1.2d. XRPD patterns were acquired under ambient conditions via a transmission foil sample stage (polyimide—Kapton, 12.7 μm thickness film) under ambient conditions using a PANalytical X'Pert PRO. The data collection range was 2.994-35°2θ with a continuous scan speed of 0.202004° s−1.

### Differential Scanning Calorimetry (DSC)

DSC data were collected on a PerkinElmer Pyris 6000 DSC equipped with a 45-position sample holder. The instrument was verified for energy and temperature calibration using certified indium. A predefined amount of the sample, 0.5-3.0 mg, was placed in a pin holed aluminium pan and heated at 20° C.min−1 from 30 to 350° C. or varied as experimentation dictated. A purge of dry nitrogen at 20 ml min−1 was maintained over the sample. The instrument control, data acquisition and analysis were performed with Pyris Software v11.1.1 revision H.

### Thermo-Gravimetric Analysis (TGA)

TGA data were collected on a Perkin Elmer Pyris 1 TGA equipped with a 20-position auto-sampler. The instrument was calibrated using a certified weight and certified Alumel and Perkalloy for temperature. A predefined amount of the sample, 1-5 mg, was loaded onto a pre-tared aluminium crucible and was heated at 20° C.min−1 from ambient temperature to 400° C. A nitrogen purge at 20 ml·min−1 was maintained over the sample. Instrument control, data acquisition and analysis was performed with Pyris Software v11.1.1 revision H.

### Gravimetric Vapour Sorption (GVS)

GVS studies were carried out on salts of the invention using the protocol set out below:

Sorption isotherms were obtained using a Hiden Isochema moisture sorption analyser (model IGAsorp), controlled by IGAsorp Systems Software V6.50.48. The sample was maintained at a constant temperature (25° C.) by the instrument controls. The humidity was controlled by mixing streams of dry and wet nitrogen, with a total flow of 250 ml·min−. The instrument was verified for relative humidity (RH) content by measuring three calibrated Rotronic salt solutions (10-50-88%). The weight change of the sample was monitored as a function of humidity by a microbalance (accuracy+/−0.005 mg). A defined amount of sample was placed in a tared mesh stainless steel basket under ambient conditions. A full experimental cycle typically consisted of three scans (sorption, desorption and sorption) at a constant temperature (25° C.) and 10% RH intervals over a 0-90% range (60 minutes for each humidity level). This type of experiment should demonstrate the ability of samples studied to absorb moisture (or not) over a set of well-determined humidity ranges.

### HPLC Method 1

HPLC analysis was carried out on an Agilent 1110 series HPLC system. The column used was an Aquity BEH Phenyl; 30×4.6 mm, 1.7 μm particle size (Ex Waters, PN: 186004644). The flow rate was 2.0 mL/min. Mobile phase A was Water:Trifluoroacetic acid (100:0.03%) and mobile phase B was Acetonitrile:Trifluoroacetic acid (100:0.03%). Detection was by UV at 210 nm. The injection volume was 5 μL and the following gradient was used:

| Time | % A | % B |
|---|---|---|
| 0 | 95 | 5 |
| 5.2 | 5 | 95 |
| 5.7 | 5 | 95 |
| 5.8 | 95 | 5 |
| 6.2 | 95 | 5 |

### HPLC Method 2

HPLC analysis was carried out on an Agilent 1110/1200 series HPLC system. The column used was an Triart C18; 150×4.6 mm, 3.0 μm particle size (Ex Waters, PN: 186004644). The flow rate was 1.0 mL/min. Mobile phase A was Water:Trifluoroacetic acid (100:0.1%) and mobile phase B was Acetonitrile:Trifluoroacetic acid (100:0.1%). Detection was by UV at 302 nm. The injection volume was 5 μL, column temperature 40° C. and the following gradient was used:

| Time (min) | % A | % B |
|---|---|---|
| 0 | 95 | 5 |
| 5 | 65 | 35 |
| 10 | 65 | 35 |
| 18 | 5 | 95 |
| 22.5 | 5 | 95 |
| 23 | 95 | 5 |

### Example 1

### Preparation and characterisation of 5-[[5-[4-(4-fluoro-1-methyl-4-piperidyl)-2-methoxy-phenyl]-1H-pyrazol-3-yl]amino]pyrazine-2-carbonitrile free base

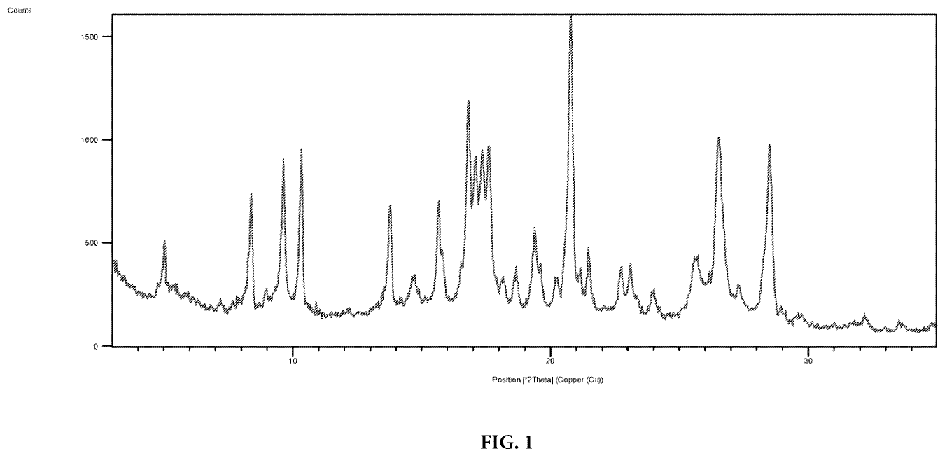

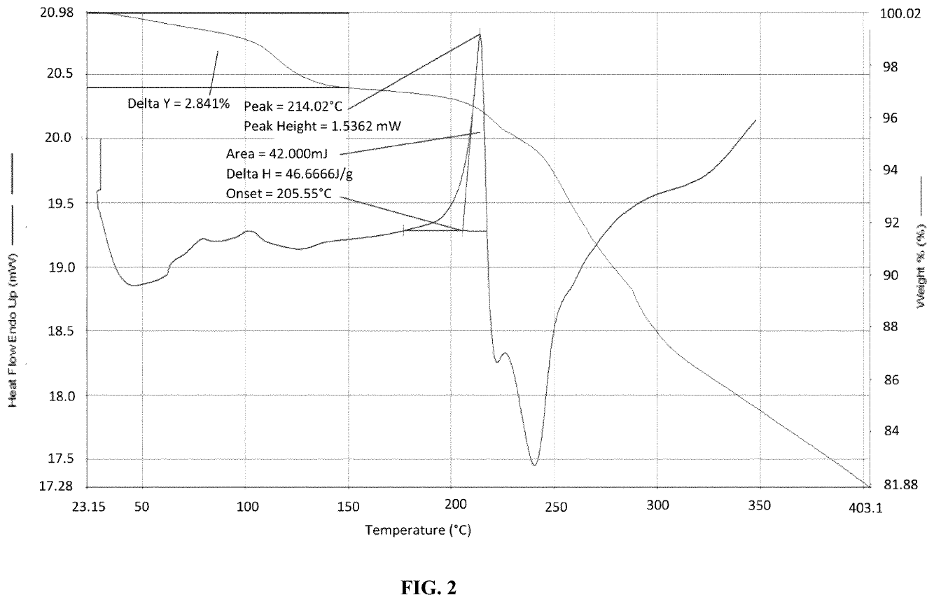

The title compound was prepared by the method of Example 64, Method L in WO 2015/20390 (the contents of which are incorporated herein by reference) but isolating the compound as the free base rather than the hydrochloric acid salt. The free base was characterised by X-Ray Powder Diffraction (XRPD), Differential Scanning Calorimetry (DSC) and Thermogravimetric analysis (TGA). The XRPD spectrum and the DSC and TGA traces are shown in FIGS. 1 to 2.

The free base was shown to be crystalline by XRPD. The DSC thermograph shows a main melt endotherm with an onset temperature of 205.6° C. and a peak temperature of 214° C. The TGA thermograph shows a weight reduction of 2.8% up to 150° C. The ¹H NMR spectrum of the solid conforms to the molecular structure. As there is no significant solvent present in the NMR spectrum, the weight loss shown in the TGA thermograph relates to the loss of water as the material is heated.

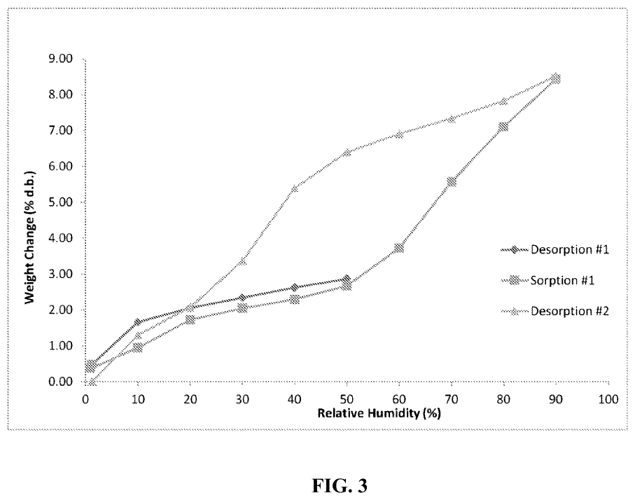

The GVS profile of the free base is shown in FIG. 3. During the initial desorption cycle the solid loses 2 wt % from 50% relative humidity (RH) to 0% RH. During the subsequent sorption cycle, the solid gains 8% of water up to 90% RH. The water uptake is reversible with hysteresis noted. The theoretical water content for a formal monohydrate of the freebase is 4.2%, so water is absorbed up to a dihydrate level at extremes of humidity.

### Example 2

### Preparation of the Salts

### Small Scale Methods

Acid addition salts of 5-[[5-[4-(4-fluoro-1-methyl-4-piperidyl)-2-methoxy-phenyl]-1H-pyrazol-3-yl]amino]pyrazine-2-carbonitrile were prepared from the free base by the small scale Methods 1 to 7 below.

### Method 1: THF Mediated

The free base of 5-[[5-[4-(4-fluoro-1-methyl-4-piperidyl)-2-methoxy-phenyl]-1H-pyrazol-3-yl]amino]pyrazine-2-carbonitrile (50 mg) was charged into 16 crystallisation tubes. THF (2 mL, 40 vols) was added, and the resulting mixtures were heated to 60° C. Acids (1M, 1 eq) were charged in one single aliquot. The solutions were held at temperature and equilibrated for 1 hour. The solutions were then cooled to room temperature and equilibrated for 18 hours before isolation by filtration and drying in vacuo for 18 hours. In several cases where crystallisation did not occur, the samples were manipulated further by removal of solvent using nitrogen and trituration of the solids with MeOH. The following salts required solvent reduction and trituration: esylate, besylate, acetate and malonate.

### Method 2: THF:MeCN Mediated

Method 2 was identical to Method 1 except that a mixture of THF:MeCN (1:1) (1 mL, 20 vols) was used as the solvent and the mixtures were heated to 50° C. The benzenesulphonic salt required solvent reduction and trituration.

### Method 3: THF:Water Mediated

Method 3 was identical to Method 2 except that THF:water (95:5) (1 mL, 20 vols) was used as the solvent and the mixtures were heated to 50° C. The benzenesulfonic, acetic, L-glutamic and L-aspartic acid salts required solvent reduction and trituration.

### Method 4: THF Mediated Using Excess Acid

Method 4 was identical to Method 1 except that acids (1M, 1.84 eq) were charged in one single aliquot. The following salts were isolated using this method: hydrochloride pattern A, hemi-fumarate pattern A, hydrobromide pattern C, bis-mesylate pattern A, bis-maleate pattern A, bis-besylate pattern A, tosylate pattern C and acetate pattern B

### Method 5: THF:Water Mediated Using Excess Acid

Method 5 was identical to Method 2 except that acids (1M, 1.84 eq) were charged in one single aliquot. The following salts were isolated using this method: L-tartrate pattern B, tosylate pattern A, phosphate pattern B, citrate pattern B, acetate pattern B, L-glutamate pattern A, hydrochloride pattern D, hydrobromide pattern D, bis-mesylate pattern B, bis-maleate pattern B, besylate pattern B, sulphate pattern C.

### Method 6: THF Mediated Using Excess Acid

Method 6 was identical to Method 1 except that the free base (30 mg) and the acids (1M, 2 e.q.) were charged in one single aliquot. This method was used to make the hydrochloride pattern B salt.

### Method 7: THF Mediated Using 0.5 Equivalents of Acid

Method 7 was identical to Method 1 except that 0.5 equivalents of acid were added in each case. The following salts were isolated using Method 7: Hemi-maleate pattern A and hemi-sulphate pattern A, hemi-ethane-1,2-disulfonate pattern A and hemi-naphthylene-1,5-disulphonate pattern A.

### Medium Scale Preparation of 5-[[5-[4-(4-fluoro-1-methyl-4-piperidyl)-2-methoxy-phenyl]-1H-pyrazol-3-yl]amino]pyrazine-2-carbonitrile salts

### Medium Scale Method 1

Larger scale preparations of salts were carried out using similar conditions to those used in small scale Method 1 but with 300 mg of free base. The following salts were prepared in this way.
Tosylate pattern C, thermal cycling at >200° C. afforded tosylate pattern D
Maleate pattern A, conditioning at 40° C./75% RH afforded maleate pattern B
Besylate pattern B
Naphthylene-2-sulfonate pattern A

This method was modified by using 100 mg of free base to form:
Oxalate pattern A

### Medium Scale Method 2

The preparation of malonate pattern B was scaled up following the method below:

5-[[5-[4-(4-fluoro-1-methyl-4-piperidyl)-2-methoxy-phenyl]-1H-pyrazol-3-yl]amino]pyrazine-2-carbonitrile free base (300 mg) was weighed into a 25 mL round bottom flask. THF:water (95:5, 20 vol) was added and the mixture was equilibrated at 60° C. for 15 min. Malonic acid (1 eq.) was charged and the mixture was equilibrated at 60° C. for 15 minutes. The mixture was cooled to room temperature and equilibrated for 30 minutes. The mixture was then rapidly evaporated using a rotary evaporator at 50° C. and 210 rpm. This resulted in the production of a beige powder that was subsequently matured in MeOH (20 vol) at room temperature for 18 hours. The resulting suspension was isolated using vacuum filtration and the solid dried in vacuo at 45° C. over the weekend.

### Medium Scale Method 3

The conditions used in small scale Method 5 were used except that 300 mg of free base and 2 equivalents of acid were used. This method was used to prepare the bis-maleate pattern A salt.

### Medium Scale Method 4

The conditions used in small scale Method 4 were used except that 300 mg of free base and 2 equivalents of acid were used. This method was used to prepare bis-besylate pattern B

### Maturation Methods

Besylate pattern C was formed following water maturation (24 h) of besylate pattern B.

Maleate pattern C was formed following water maturation (24 h) of maleate B.

A summary of the methods used to prepare the salts and the physical appearances of the salts thus prepared is shown in the Table below.

TABLE
Preparation of salts

| Salt | Method | Appearance |
|---|---|---|
| Hydrochloride (XRPD pattern A) | 2, 4 | Pale yellow solid |
| Hydrochloride (XRPD pattern B) | 6 | Off-white solid |
| Hydrochloride (XRPD pattern C) | 1 | Off-white solid |
| Hydrochloride (XRPD pattern D) | 5 | Off-white solid |
| Hydrochloride (XRPD pattern E) | 3 | Light yellow solid |
| Hydrobromide (XRPD pattern A) | 1, 3 | Light yellow solid |
| Hydrobromide (XRPD pattern B) | 2 | Off-white solid |
| Hydrobromide (XRPD pattern C) | 4 | Off-white solid |
| Hydrobromide (XRPD pattern D) | 5 | Off-white solid |
| Mesylate (XRPD pattern A) | 1 | Off-white solid |
| Mesylate (XRPD pattern B) | 2 | Off-white solid |
| Mesylate (XRPD pattern C) | 3 | Off-white solid |
| L-Tartrate (XRPD pattern A) | 2 | Off-white solid |
| L-Tartrate (XRPD pattern B) | 3, 5 | Off-white solid |
| Esylate (XRPD pattern A) | 1 | Off-white solid |
| Esylate (XRPD pattern B) | 2, 3 | Off-white solid |
| L-Aspartate (XRPD pattern A) | 3 | Off-white solid |
| Besylate (XRPD pattern A) | 1, 2, 3 | Off-white solid |
| Besylate (XRPD pattern B) | 5, 1(scale-up) | Off-white solid |
| Besylate (XRPD pattern C) | Water maturation | |
| | of besylate B | |
| Tosylate (XRPD pattern A) | 1, 3, 5 | Off-white solid |
| Tosylate (XRPD pattern B) | 2 | Off-white solid |
| Tosylate (XRPD pattern C) | 1(scale-up), 4 | Off-white solid |
| Tosylate (XRPD pattern D) | Thermal cycling | |
| | of pattern C | |
| Sulfate (XRPD pattern A) | 1, 2 | Pale yellow solid |
| Sulfate (XRPD pattern B) | 3 | Pale yellow solid |
| Sulphate (XRPD pattern C) | 5 | Off-white solid |
| Sulphate (XRPD pattern D) | 7 | Off-white solid |
| Sulphate (XRPD pattern E) | 7 | Off-white solid |
| Phosphate (XRPD pattern A) | 1, 2 | Off-white solid |
| Phosphate (XRPD pattern B) | 3, 5 | Off-white solid |
| Citrate (XRPD pattern A) | 2 | Off-white solid |
| Citrate (XRPD pattern B) | 3, 5 | Off-white solid |
| Acetate (XRPD pattern A) | 1, 3 | Off-white solid |
| Acetate (XRPD pattern B) | 5 | Off-white solid |
| L-glutamate (XRPD pattern A) | 3,5 | Off-white solid |
| Maleate (XRPD pattern A) | 1, 2, 3 | Off-white solid |
| Maleate (XRPD pattern B) | Conditioning of | Light brown |
| | maleate A at | to yellow |
| | 40° C./75% RH | solid |
| Maleate (XRPD pattern C) | Water maturation | Light brown |
| | of maleate B | to yellow |
| | | solid |
| Gentisate (XRPD pattern A) | 1, 3 | Off-white solid |
| Glucuronate (XRPD pattern A) | 1 | Pale yellow solid |
| Glucuronate (XRPD pattern B) | 3 | Pale yellow solid |
| Malonate (XRPD pattern A) | 1 | Off-white solid |
| Malonate (XRPD pattern B) | 3 | Off-white solid |
| Naphthylene-2-sulphonate | 1, 3 | Off-white solid |
| (XRPD pattern A) | | |
| Oxalate (XRPD pattern A) | 1 | Off-white solid |
| Oxalate (XRPD pattern B) | 3 | Off-white solid |

The characterising data for the salts prepared according to the methods described above are set out in the table below.

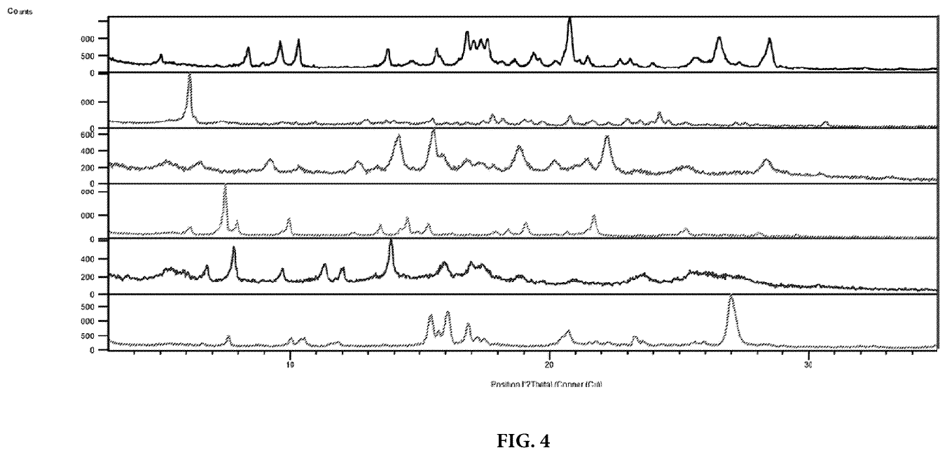

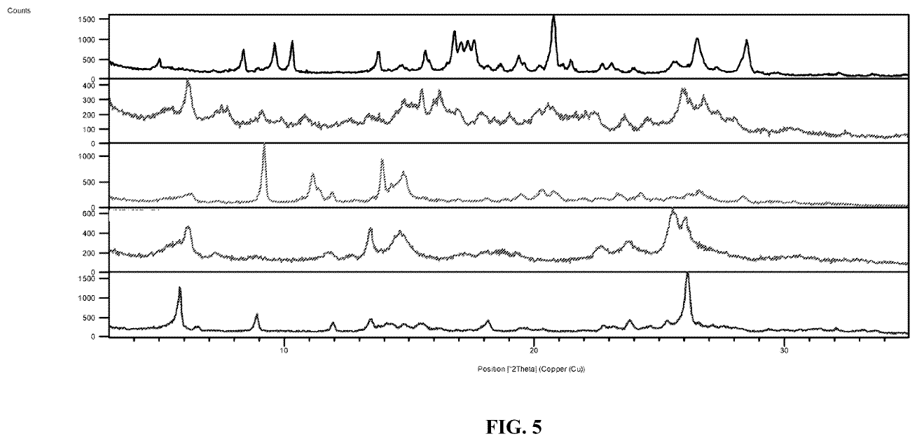

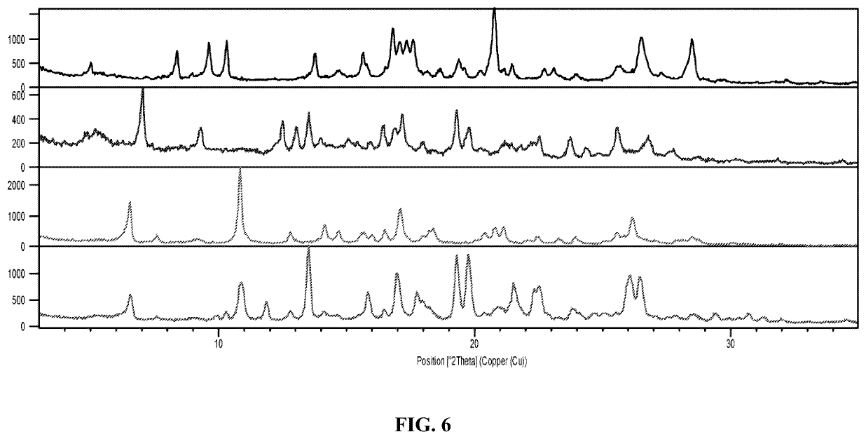

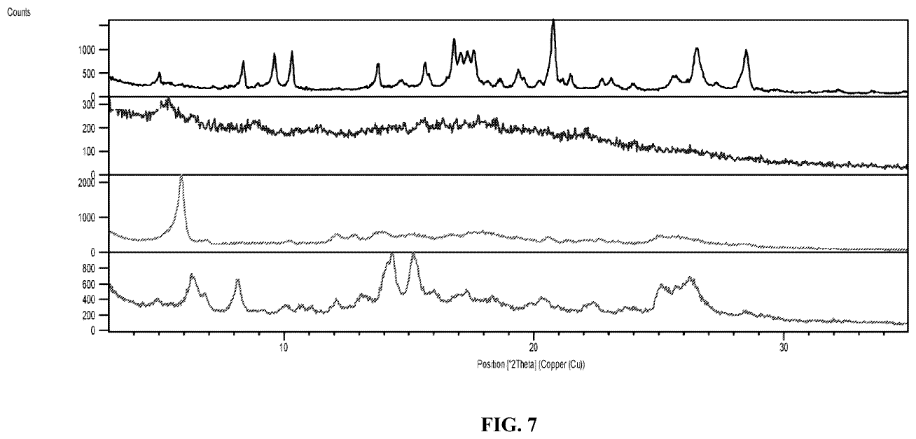

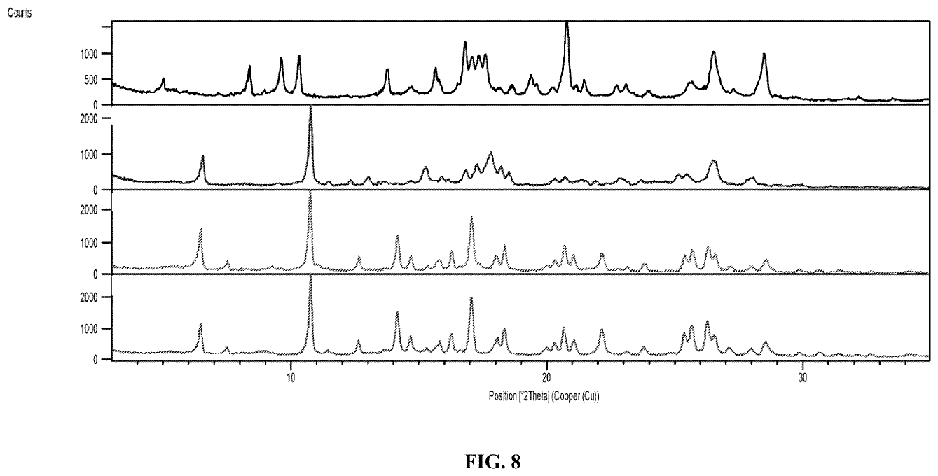

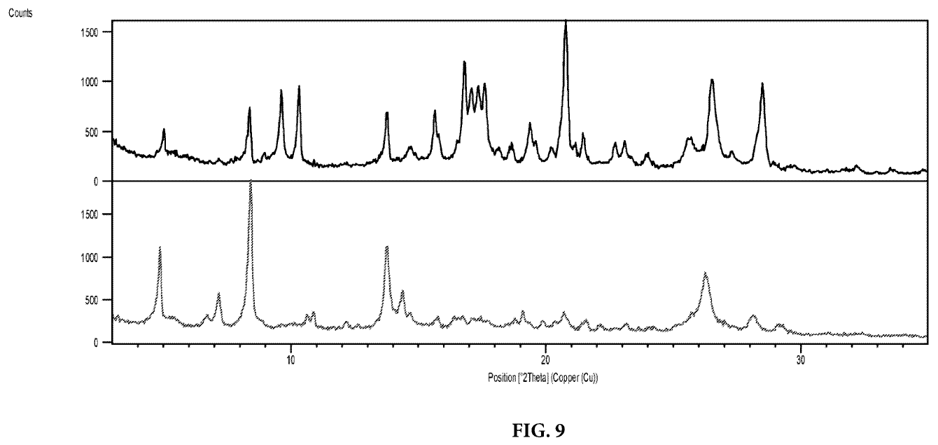

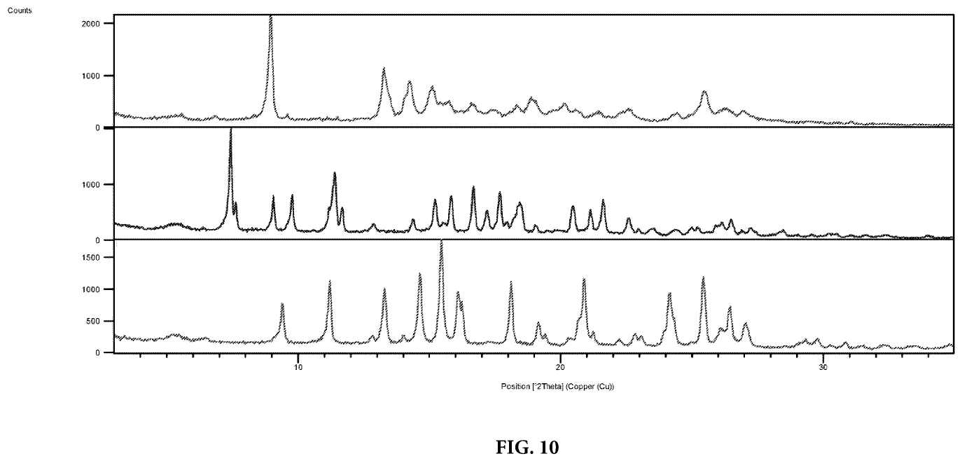

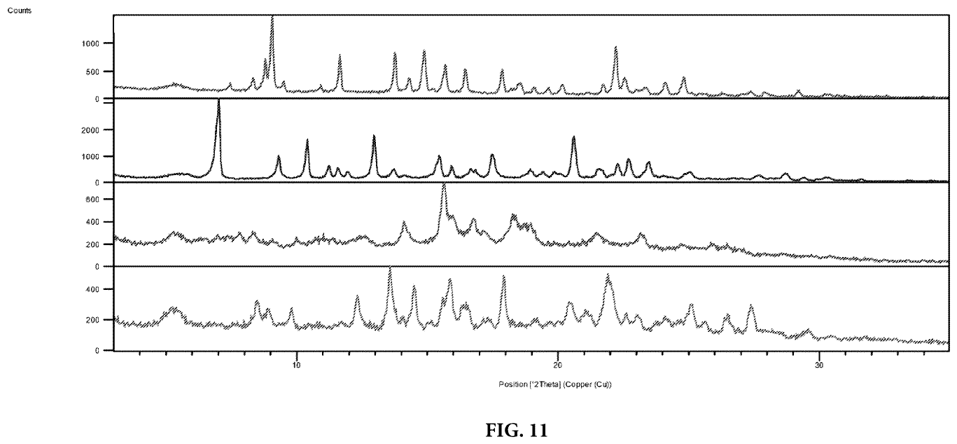

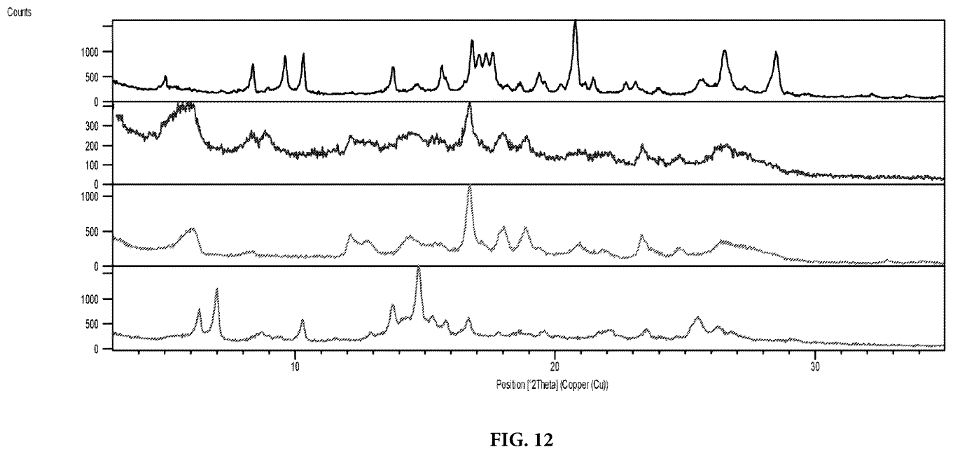

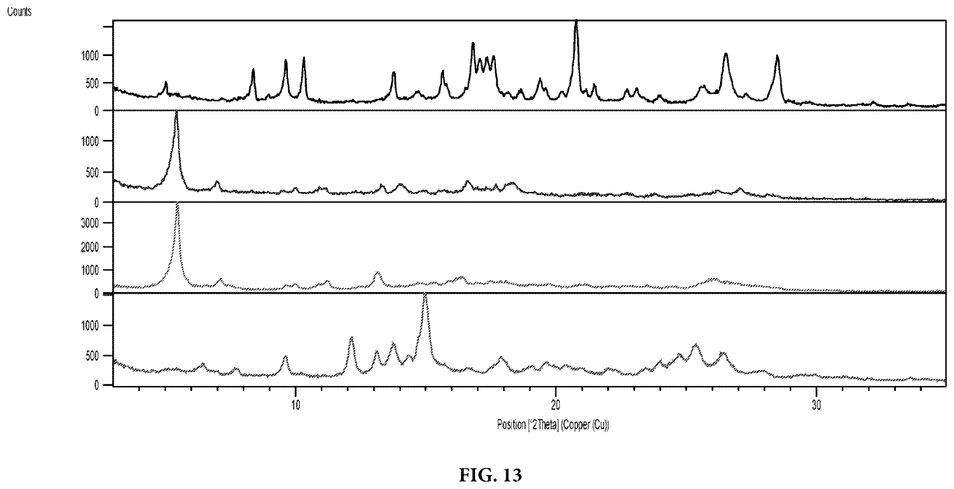

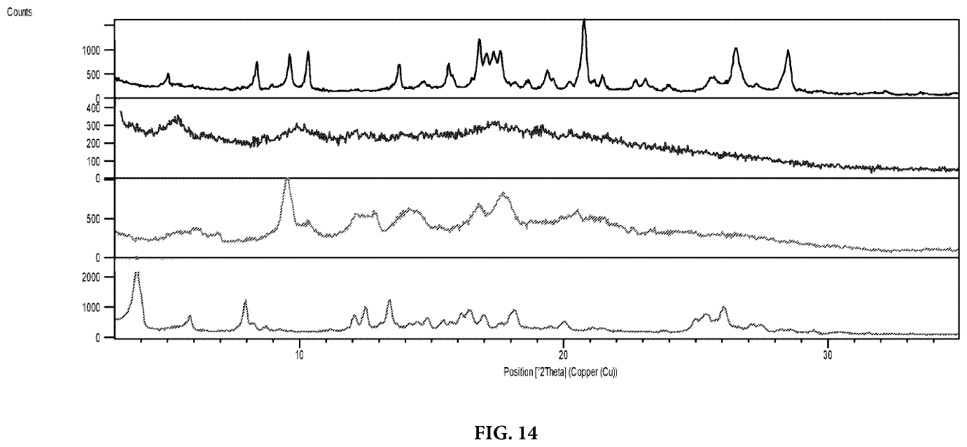

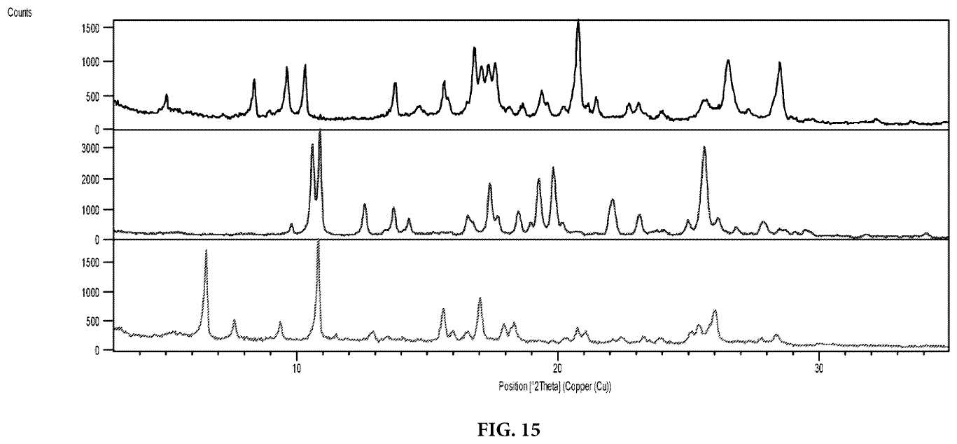

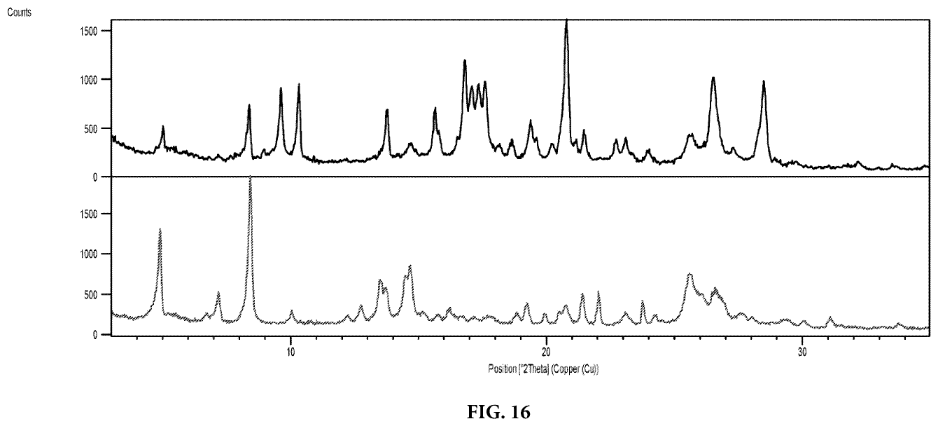

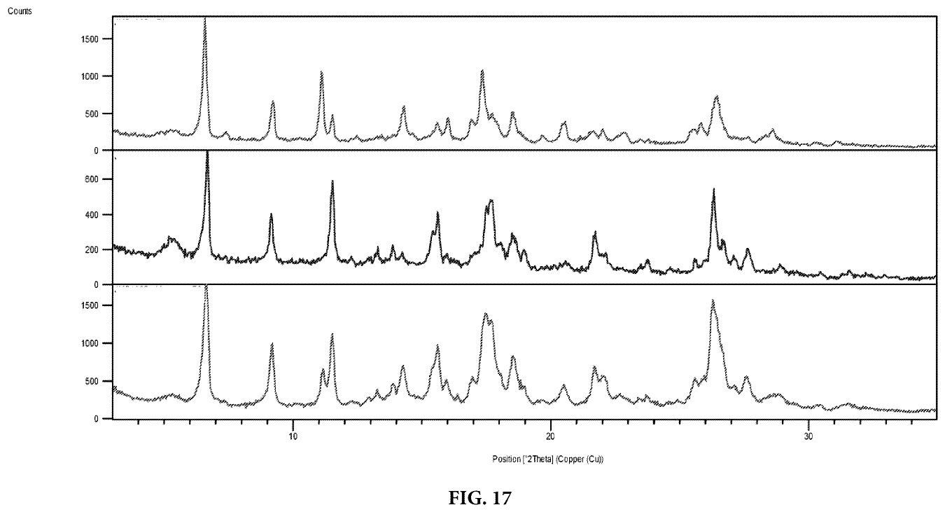

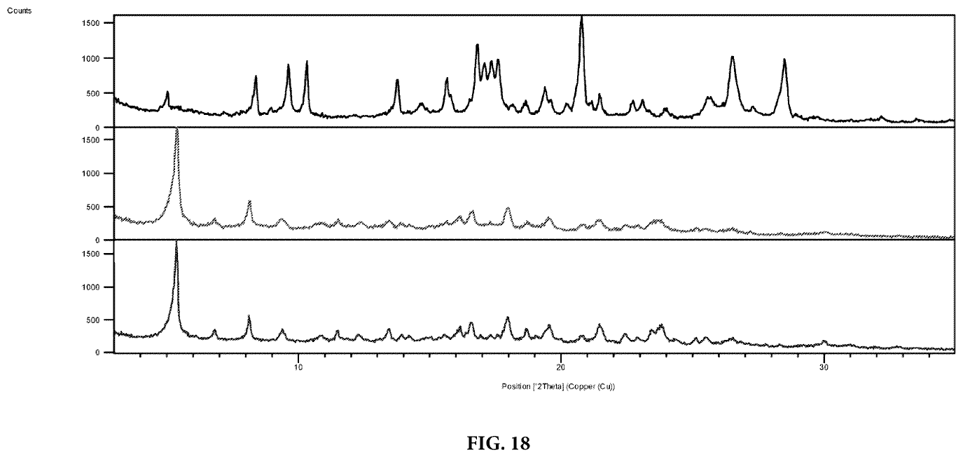

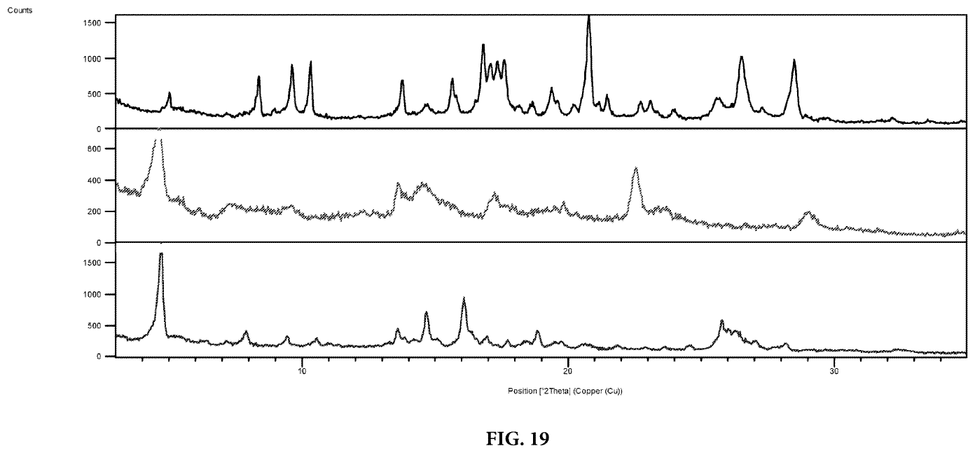

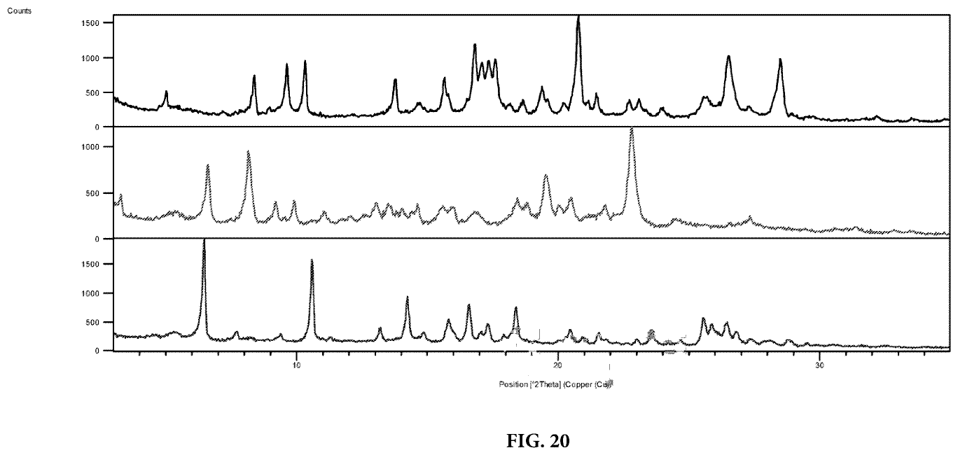

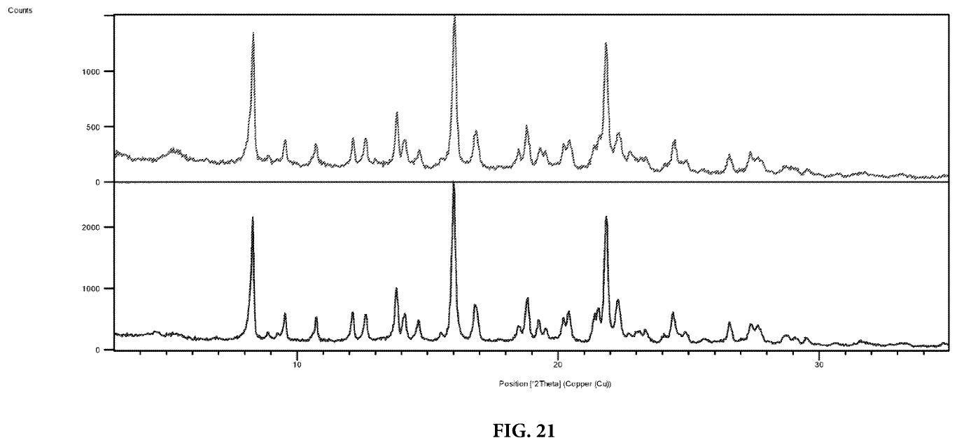

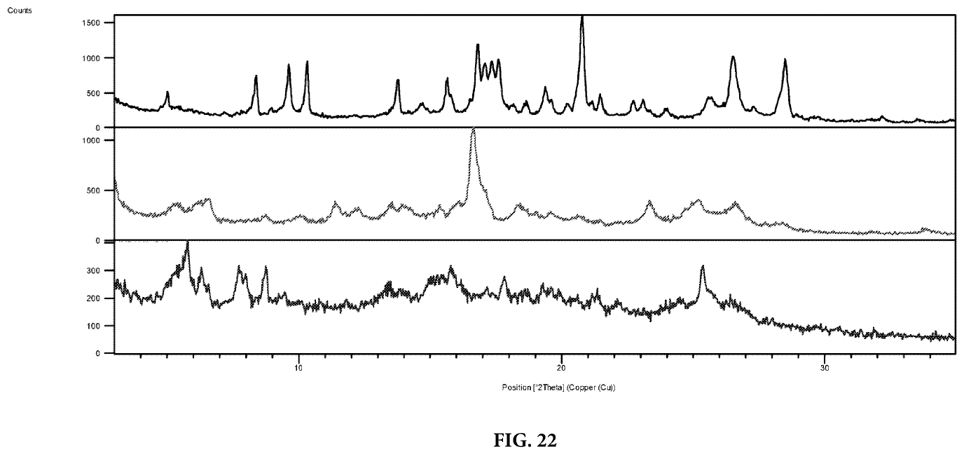

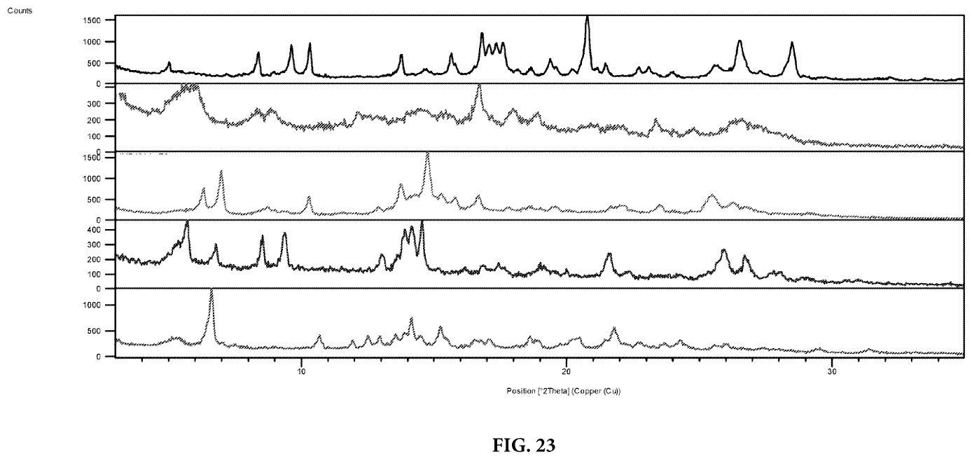

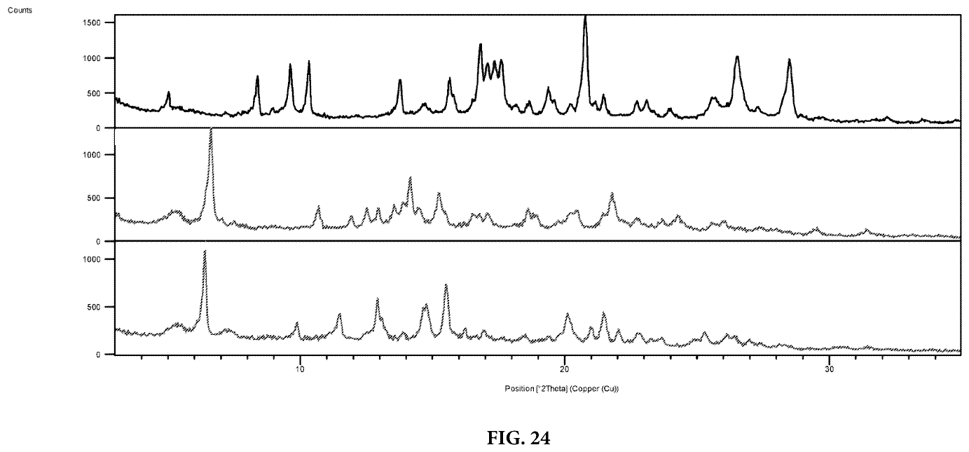

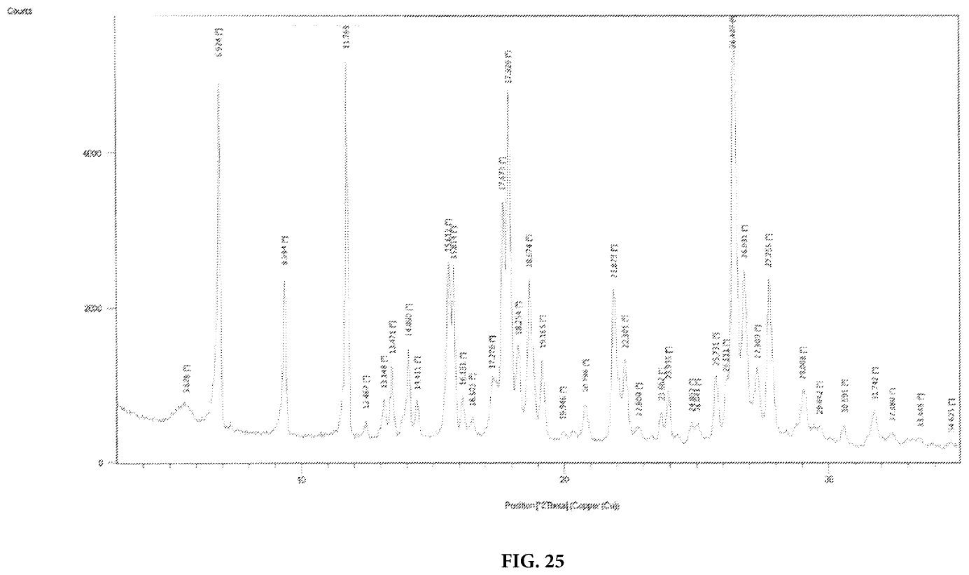

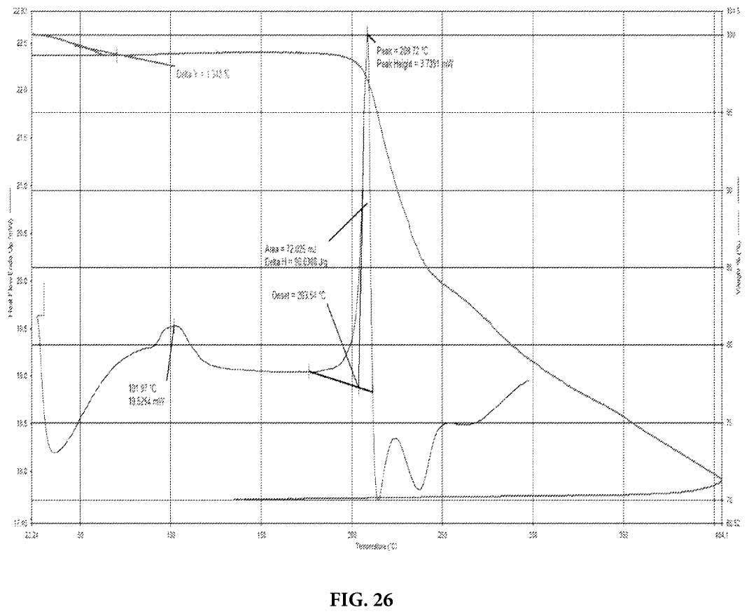

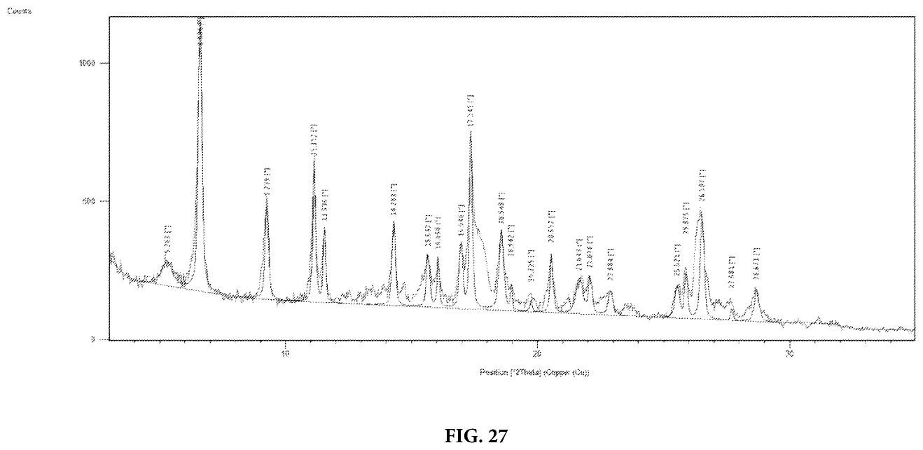

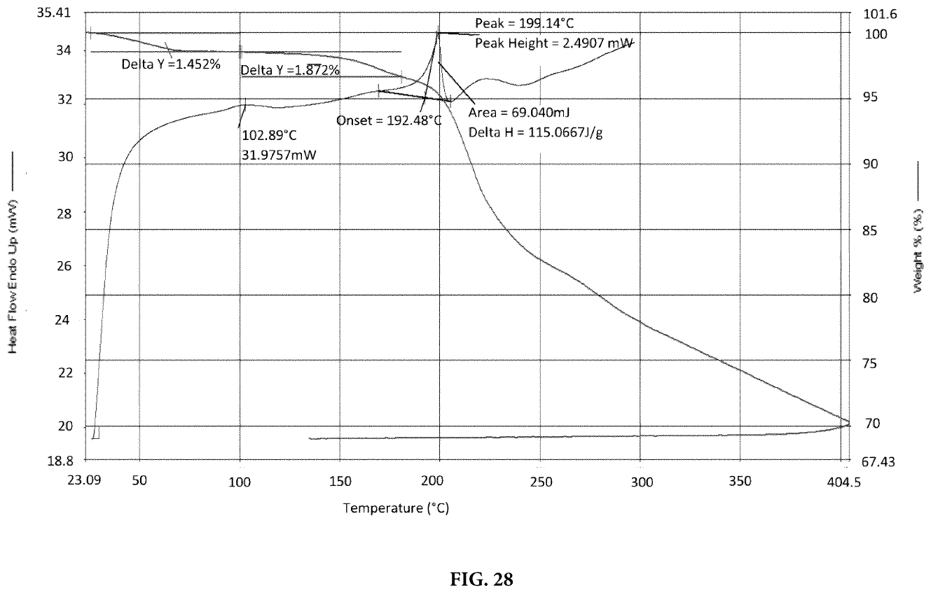

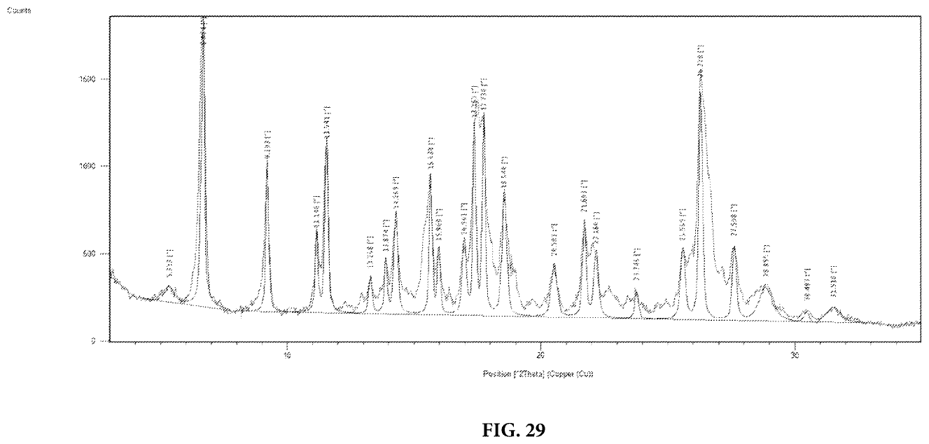

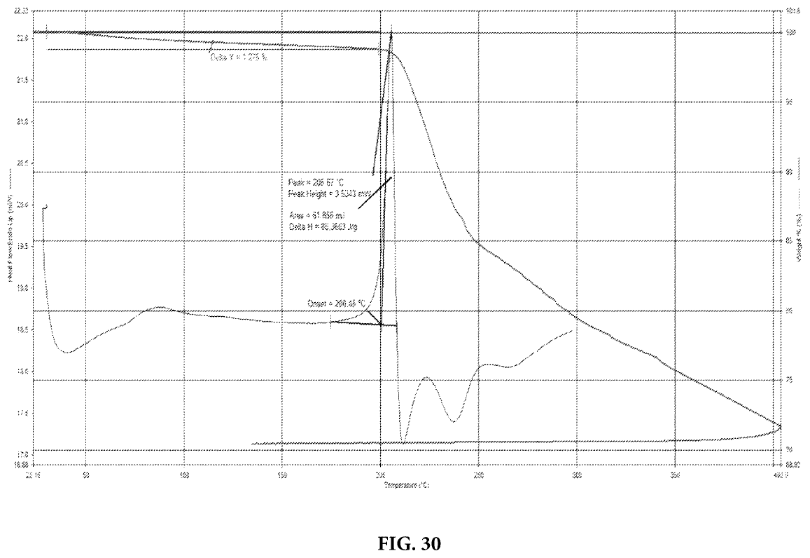

TABLE
Characterising Data for the Salts

| | XRPD | Thermal profile | |
|---|---|---|---|
| Salt | FIGURE | DSC/TGA | NMR |
| Hydrochloride | FIG. 4 | DSC events: | ¹H NMR confirms |
| (XRPD pattern A) | second | Endotherms at | salt formation, IC |
| | from | 169º C. and 285°, | required to confirm |
| | top trace | exotherm at | stoichiometry |
| | | 219° C. | |
| Hydrochloride | FIG. 4 | DSC events: | ¹H NMR confirms |
| (XRPD pattern B) | third from | Broad endotherm | salt formation, IC |
| | top | at 220° C. | required to confirm |
| | second | | stoichiometry |
| Hydrochloride | FIG. 4 | DSC events: | ¹H NMR confirms |
| (XRPD pattern C) | third from | Endotherms at | salt formation, IC |
| | bottom | 172° C. and | required to confirm |
| | trace | 209° C. | stoichiometry |
| Hydrochloride | FIG. 4 | DSC events: small | ¹H NMR confirms |
| (XRPD pattern D) | second | endotherms at | salt formation, IC |
| (prepared using | from | 140 and 214° C. | required to confirm |
| excess acid) | bottom | TGA events: loss | stoichiometry |
| | trace | of 3.8% up to | |
| | | 80° C. then loss of | |
| | | 3.9% coinciding | |
| | | with first | |
| | | endotherm | |
| Hydrochloride | FIG. 4 | DSC events: 143, | ¹H NMR confirms |
| (XRPD pattern E) | bottom | 174, 196 and | salt formation, IC |
| | trace | 217° C. | required to confirm |
| | | (main melt) | stoichiometry |
| | | TGA events: loss | |
| | | of 3.1% up to | |
| | | 150° C. | |
| Hydrobromide | FIG. 5 | DSC events: | ¹H NMR confirms |
| (XRPD pattern A) | second | onset 205° C., peak | salt formation, IC |
| | from | 209° C. | required to confirm |
| | top trace | TGA events: loss | stoichiometry |
| | | of 2.3% up to | |
| | | 100° C. and 3.6% | |
| | | over main melt | |
| Hydrobromide | FIG. 5 | DSC events: small | ¹H NMR confirms |
| (XRPD pattern B) | third | endotherm 180° C. | salt formation, IC |
| | from | then 209° C. and | required to confirm |
| | top trace | broad endotherm | stoichiometry |
| | | 270° C. | |
| | | TGA events: loss | |
| | | of 4.7% up to | |
| | | 100° C. and loss | |
| | | of 3.7% during | |
| | | second | |
| | | endotherm | |
| Hydrobromide | FIG. 5 | DSC events: | ¹H NMR confirms |
| (XRPD pattern C) | second | shouldered peak | salt formation, IC |
| (prepared using | from | at 247° C. | required to confirm |
| excess acid) | bottom | TGA events: loss | stoichiometry |
| | trace | of 4.4% up to | |
| | | 150° C. | |
| Hydrobromide | FIG. 5 | DSC events: | ¹H NMR confirms |
| (XRPD pattern D) | bottom | broad endotherm | salt formation, IC |
| (prepared using | trace | at 252° C. | required to confirm |
| excess acid) | | TGA events: loss | stoichiometry |
| | | of 7.3% up to | |
| | | 150° C. | |
| Mesylate (XRPD | FIG. 6 | DSC events: | ¹H NMR confirms |
| pattern A) | second | Peaks at 195 and | mono |
| | from top | 209° C. | stoichiometry |
| | trace | TGA events: loss | |
| | | of 1.6% up to | |
| | | 100° C. and 4.6% | |
| | | over main | |
| | | endotherm 150- | |
| | | 250° C. | |
| Mesylate (XRPD | FIG. 6 | DSC events: | ¹H NMR confirms |
| pattern B) | second | peaks at 164 and | mono |
| | from | 210° C. | stoichiometry |
| | bottom | TGA events: loss | |
| | trace | of 4.2% up to | |
| | | 100° C. then 2.25 | |
| | | and 2.9% | |
| | | coinciding with | |
| | | endotherms | |
| Mesylate (XRPD | FIG. 6 | DSC events: | ¹H NMR confirms |
| pattern C) | bottom | peaks at 171, | mono |
| | trace | 200° C. | stoichiometry |
| | | TGA events: loss | |
| | | of 4.6% up to | |
| | | 150° C., then 2.8% | |
| | | between 200 and | |
| | | 250° C. | |
| L-Tartrate (XRPD | FIG. 7 | DSC events: | ¹H NMR confirms |
| pattern A) | middle | minor 145° C., | mono |
| | two | broad endotherm | stoichiometry |
| | traces | 200° C. | |
| | | TGA events: Loss | |
| | | of 1.6% up to | |
| | | 100° C. and 0.8% | |
| | | loss between 175 | |
| | | and 225° C. | |
| L-Tartrate (XRPD | FIG. 7 | DSC events: | ¹H NMR confirms |
| pattern B) | bottom | minor 155° C., | mono |
| | trace | broad endotherm | stoichiometry |
| | | 200° C. | |
| | | TGA events: 4.3% | |
| | | loss up to 150° C. | |
| Esylate (XRPD | FIG. 8 | DCS events: | ¹H NMR confirms |
| pattern A) | second | 145° C. | mono |
| | from top | and 215° C. | stoichiometry |
| | trace | TGA events: loss | |
| | | of 1.8% up to | |
| | | 100° C. then 2.4% | |
| | | over first | |
| | | endotherm and | |
| | | 2.8% over second | |
| | | endotherm | |
| Esylate (XRPD | FIG. 8 | DSC events: | ¹H NMR confirms |
| pattern B) | bottom | broad endotherms | mono |
| | two | 153 and 192° C. | stoichiometry |
| | traces | TGA events: loss | |
| | | of 4.8% up to | |
| | | 100° C., 1.4% loss | |
| | | up to 175° C. | |
| L-Aspartate | FIG. 9 | DSC events: | ¹H NMR confirms |
| (XRPD pattern A) | bottom | endotherms 106, | mono |
| | trace | 163, bimodal 207, | stoichiometry |
| | | 220° C. | |
| | | TGA events: loss | |
| | | of 6.9% up to | |
| | | 100° C., then 2.4% | |
| | | to 175° C. | |
| Besylate (XRPD | FIG. 10 | DSC events: | ¹H NMR confirms |
| pattern A) | top | endotherms at | mono |
| | trace | 166, 189 and | stoichiometry |
| | | 217° C. | |
| | | TGA events: loss | |
| | | of 0.8% up to | |
| | | 100° C. and 3% | |
| | | loss over second | |
| | | endotherm | |
| Besylate (XRPD | FIG. 10 | DSC events: | ¹H NMR confirms |
| pattern B) | middle | bimodal | mono |
| | trace | endotherm with | stoichiometry |
| | | peaks at 211 and | |
| | | 223° C. | |
| | | TGA events: 0.4% | |
| | | from 130° C. prior | |
| | | to main melt | |
| Besylate (XRPD | FIG. 10 | DSC events: | ¹H NMR confirms |
| pattern C) | bottom | single endotherm | mono |
| | trace | at 230° C. | stoichiometry |
| | | TGA events: loss | |
| | | of 2.1% up to | |
| | | 100° C. | |
| Tosylate (XRPD | FIG. 11 | DSC events: | ¹H NMR confirms |
| pattern A) | top | 106° C. then main | mono |
| | trace | melt 234° C. | stoichiometry |
| | | TGA events: loss | |
| | | of 2.7% up to | |
| | | 100° C. | |
| Tosylate (XRPD | FIG. 11 | DSC events: | ¹H NMR confirms |
| pattern B) | second | Broad endotherms | mono |
| | from | 157° C. and | stoichiometry |
| | top trace | 217° C. | |
| | | TGA events: loss | |
| | | of 2.6% up to | |
| | | 100° C., then 1.6 | |
| | | and 2.8% losses | |
| | | coinciding with | |
| | | endotherms | |
| Tosylate (XRPD | FIG. 11 | DSC events: | ¹H NMR confirms |
| pattern C) | second | endotherm 125° C., | mono |
| | from | exotherm 185° C. | stoichiometry |
| | bottom | and shouldered | |
| | trace | endotherm at | |
| | | 225° C. | |
| | | TGA events: loss | |
| | | of 1.5% up to | |
| | | 150° C. and loss of | |
| | | 1.6% from 100- | |
| | | 175° C. | |
| Tosylate (XRPD | FIG. 11 | DSC events: | ¹H NMR confirms |
| pattern D) | bottom | single main melt | mono |
| | trace | at 222° C. | stoichiometry |
| | | TGA events: no | |
| | | loss of mass prior | |
| | | to main melt | |
| Sulphate (XRPD | FIG. 12 | DSC events: | ¹H NMR confirms |
| pattern A) | middle | endotherms | salt formation, IC |
| | two | 187° C., and | required to confirm |
| | traces | 267° C., exotherm | stoichiometry |
| | | at 250° C. | |
| | | TGA events: loss | |
| | | of 3.4% up to | |
| | | 150° C. and 3.3% | |
| | | loss over first | |
| | | endotherm | |
| Sulphate (XRPD | FIG. 12 | DSC events: | ¹H NMR confirms |
| pattern B) | bottom | Broad endotherms | salt formation, IC |
| | trace | 119° C., | required to confirm |
| | | 174° C. and | stoichiometry |
| | | 265° C., exotherm | |
| | | at 250° C. | |
| | | TGA events: loss | |
| | | of 3.9% up to | |
| | | 110° C., then 2.7% | |
| | | losses prior to | |
| | | exotherm | |
| Sulphate (XRPD | FIG. 23 | DSC events: | ¹H NMR confirms |
| pattern C) | middle | broad peaks at | salt formation, IC |
| (prepared from | trace | 131° C. | required to confirm |
| excess acid) | | and 179° C., | stoichiometry |
| | | sharp exotherm | |
| | | at 272° C. | |
| | | TGA events: loss | |
| | | of 9.2% up to | |
| | | 125° C. | |
| Sulphate (XRPD | FIG. 23 | DSC events: | ¹H NMR confirms |
| pattern D) | bottom | Single endotherm | salt formation, IC |
| (Prepared from 0.5 | trace | at 187° C. | required to confirm |
| eq acid | | TGA events: 0.7% | stoichiometry |
| stoichiometry not | | loss of mass up to | |
| confirmed, labelled | | 100° C. | |
| hemi-sulphate | | | |
| pattern A in | | | |
| reports) | | | |
| Sulphate (XRPD | FIG. 24 | DSC events: small | ¹H NMR confirms |
| pattern E) | bottom | endotherm at | salt formation, IC |
| (Prepared from 0.5 | trace | 201° C. | confirms mono |
| eq acid but IC | | TGA events: Loss | stoichiometry |
| indicated mono | | of 0.34% prior to | |
| salt, labelled hemi- | | melt endotherm | |
| sulphate pattern A | | | |
| in reports) | | | |
| Phosphate (XRPD | FIG. 13 | DSC events: | NMR inconclusive, |
| pattern A) | middle | endotherm 164° C. | IC required to |
| | two | TGA: Loss of | confirm |
| | traces | 2.1% prior to main | stoichiometry |
| | | melt then step | |
| | | mass loss of 10% | |
| Phosphate (XRPD | FIG. 13 | DSC events: | ¹H NMR confirms |
| pattern B) | bottom | endotherms at | salt formation, IC |
| | trace | 153 and 203° C. | required to confirm |
| | | TGA events: 3.6% | stoichiometry |
| | | loss up to 150° C. | |
| Citrate (XRPD | FIG. 14 | DSC events: | ¹H NMR confirms |
| pattern A) | second | endotherms at | mono |
| | from | 163 TGA events: | stoichiometry |
| | bottom | 1.5% loss up to | |
| | trace | 100° C. | |
| Citrate (XRPD | FIG. 14 | DSC events: | ¹H NMR confirms |
| pattern B) | bottom | bimodal | mono |
| | trace | endotherms peaks | stoichiometry |
| | | at 117 and 139° C. | |
| | | and broad | |
| | | endotherm at | |
| | | 192° C. | |
| | | TGA events: 3.1% | |
| | | loss up to 100° C. | |
| | | then 1.6% loss | |
| | | coinciding with | |
| | | first endotherm | |
| Acetate (XRPD | FIG. 15 | DSC events: | ¹H NMR confirms |
| pattern A) | middle | shouldered | mono |
| | trace | endotherm at | stoichiometry |
| | | 131° C. | |
| | | followed by | |
| | | an event at 213° C. | |
| Acetate (XRPD | FIG. 15 | DSC events: | ¹H NMR confirms |
| pattern B) | bottom | 100° C., 156° C. | mono |
| | trace | TGA events: 6.2% | stoichiometry |
| | | loss up to 100° C., | |
| | | 11.5% loss 100- | |
| | | 165° C. | |
| L-glutamate | FIG. 16 | DSC events: 96, | ¹H NMR confirms |
| (XRPD pattern A) | bottom | 151, 169 and | mono |
| | trace | 201° C. | stoichiometry |
| | | TGA events: loss | |
| | | of mass up to | |
| | | 110° C. and 3.3% | |
| | | loss from 150- | |
| | | 200° C. | |
| Maleate (XRPD | FIG. 17 | DSC and TGA- | ¹H NMR confirms |
| pattern A) | top trace | FIG. 28 | mono |
| | and | DSC: main melt | stoichiometry |
| | FIG. 27 | with peak at | |
| | | 201° C. | |
| | | TGA events: no | |
| | | loss prior to main | |
| | | melt | |
| Maleate (XRPD | FIG. 17 | DSC and TGA- | ¹H NMR confirms |
| pattern B) | middle | FIG. 26 | mono |
| | trace and | DSC: main melt | stoichiometry |
| | FIG. 25 | with peak at | |
| | | 201° C. | |
| Maleate (XRPD | FIG. 17 | DSC and TGA- | ¹H NMR confirms |
| pattern C) | bottom | FIG. 30 | mono |
| | trace and | | stoichiometry |
| | FIG. 29 | DSC events: main | |
| | | melt with peak at | |
| | | 202° C. | |
| | | TGA events: 1.2% | |
| | | loss up to 120° C. | |
| Gentisate (XRPD | FIG. 18 | DSC events: | ¹H NMR confirms |
| pattern A) | middle | shouldered | mono |
| | and | endotherm at | stoichiometry |
| | bottom | 181° C. | |
| | traces | TGA events: loss | |
| | | of 0.4% up to | |
| | | 100° C. and loss of | |
| | | 0.25% from 100- | |
| | | 165° C. | |
| Glucuronate | FIG. 19 | DSC events: | ¹H NMR confirms |
| (XRPD pattern A) | middle | single endotherm | mono |
| | trace | with peak at | stoichiometry |
| | | 166° C. | |
| | | TGA events: loss | |
| | | of 0.3% up to | |
| | | 100° C. | |
| Glucuronate | FIG. 19 | DSC events: | ¹H NMR confirms |
| (XRPD pattern B) | bottom | single endotherm | mono |
| | trace | with peak at | stoichiometry |
| | | 159° C. | |
| | | TGA events: loss | |
| | | of 2.6% up to | |
| | | 100° C. and loss of | |
| | | 1% from 100- | |
| | | 130° C. | |
| Malonate (XRPD | FIG. 20 | DSC events: | ¹H NMR confirms |
| pattern A) | middle | single endotherm | mono |
| | trace | with peak at | stoichiometry |
| | | 140° C. | |
| | | TGA events: loss | |
| | | of 0.5% up to | |
| | | 100° C. | |
| Malonate (XRPD | FIG. 20 | DSC events: | ¹H NMR confirms |
| pattern B) | bottom | single endotherm | mono |
| | trace | with peak at | stoichiometry |
| | | 165° C. | |
| | | TGA events: loss | |
| | | of 0.5% up to | |
| | | 100° C. | |
| Naphthylene-2- | FIG. 21 | DSC events: high | ¹H NMR confirms |
| sulfonate (XRPD | both | melt endotherm | mono |
| pattern A) | traces | with peak of | stoichiometry |
| | | 243° C. | |
| | | TGA events: 0.8% | |
| | | loss up to 100° C. | |
| Oxalate (XRPD | FIG. 22 | DSC events: | ¹H NMR confirms |
| pattern A) | middle | endotherm with | salt formation, IC |
| | trace | peak of 200° C. | required to confirm |
| | | TGA events: 0.1% | stoichiometry |
| | | loss up to 100° C. | |
| Oxalate (XRPD | FIG. 22 | DSC events: | ¹H NMR confirms |
| pattern B) | bottom | broad endotherm | salt formation, IC |
| | trace | at 190° C. | required to confirm |
| | | TGA events: 1.6% | stoichiometry |
| | | loss up to 100° C. | |
| | | and loss of 1.6% | |
| | | from 120-170° C. | |

The characteristics of bis salts and hemi salts prepared using the methods described above are also described below.

| | | Thermal | | | |
|---|---|---|---|---|---|
| | XRPD | profile | | Method of | |
| Salt | Figure | DSC/TGA | NMR | preparation | Appearance |
| Bis- | FIG. | DSC | ¹H NMR | 4 | Off-white |
| mesylate | 37 | events: | confirms | | powder |
| (XRPD | middle | single | salt | | |
| pattern A) | trace | endotherm | formation | | |
| | | with peak | and bis- | | |
| | | at 173° C. | stoichi- | | |
| | | TGA | ometry | | |
| | | events: | | | |
| | | loss of | | | |
| | | 4.3% up to | | | |
| | | 125° C. | | | |
| Bis- | FIG. | DSC | ¹H NMR | 4 | Off-white |
| mesylate | 37 | events: | confirms | | powder |
| (XRPD | bottom | bimodal | salt | | |
| pattern B) | trace | endotherm | formation | | |
| | | with peaks | and bis- | | |
| | | at 132 and | stoichi- | | |
| | | 146° C. | ometry | | |
| | | prior to | | | |
| | | endotherm | | | |
| | | at 169° C. | | | |
| | | TGA | | | |
| | | events: | | | |
| | | loss of | | | |
| | | 3.4% up to | | | |
| | | 80° C. | | | |
| | | followed by | | | |
| | | 2.9% over | | | |
| | | bimodal | | | |
| | | endotherm | | | |
| Bis- | FIG. | DSC | ¹H NMR | 4 | Off-white |
| maleate | 38 | events: | indicates | | powder |
| (XRPD | middle | Main melt | 1:1.75 | | |
| pattern A | trace | endotherm | stoichi- | | |
| bis- | | at 185° C. | ometry | | |
| maleate) | | TGA | (mixed | | |
| | | events: | mono/bis | | |
| | | Loss of | phase) | | |
| | | 1.5% up to | | | |
| | | 100° C. | | | |
| Bis- | FIG. | DSC | ¹H NMR | 4 | Off-white |
| maleate | 38 | events: | confirms | | powder |
| (XRPD | bottom | Main melt | salt | | |
| pattern B | trace | endotherm | formation | | |
| bis- | | at 191° C. | and bis- | | |
| maleate) | | TGA | stoichi- | | |
| | | events: | ometry | | |
| | | Loss of | | | |
| | | 1.3% up to | | | |
| | | 100° C. | | | |
| Bis- | FIG. | DSC | ¹H NMR | 4 | Off-white |
| besylate | 39 | events: | confirms | | powder |
| (XRPD | middle | single | salt | | |
| pattern A) | trace | endotherm | formation | | |
| | | at 212° C. | and bis- | | |
| | | TGA | stoichi- | | |
| | | events: | ometry | | |
| | | loss of | | | |
| | | 0.8% up to | | | |
| | | 100° C. | | | |
| Bis- | FIG. | DSC | ¹H NMR | 4 | Off-white |
| besylate | 39 | events: | confirms | (scale-up) | powder |
| (XRPD | bottom | single | salt | | |
| pattern B) | trace | sharp | formation | | |
| | | endotherm | and bis- | | |
| | | at 217° C. | stoichi- | | |
| | | TGA | ometry | | |
| | | events: | | | |
| | | loss of | | | |
| | | 0.25% from | | | |
| | | 130° C. up | | | |
| | | to 200° C. | | | |

### Hemi-Salts

| | | Thermal | | | |
|---|---|---|---|---|---|
| | XRPD | profile | | Method of | |
| Salt | Figure | DSC/TGA | NMR | preparation | Appearance |
| Hemi- | FIG. | DSC | ¹H NMR | 7 | Off-white |
| maleate | 40 | events: | confirms | | solid |
| (XRPD | bottom | main melt | salt | | |
| pattern A) | trace | at 192° C. | formation | | |
| | | TGA | and hemi | | |
| | | events: | stoichi- | | |
| | | loss of | ometry. | | |
| | | 0.2% up to | THF | | |
| | | 100° C. | solvate | | |
| Hemi- | FIG. | DSC | ¹H NMR | 7 | Off-white |
| ethane-1,2- | 41 | events: | confirms | | solid |
| disulfonate | bottom | shouldered | salt | | |
| (XRPD | trace | endotherm | formation | | |
| pattern A) | | at 196° C. | and hemi- | | |
| | | TGA | stoichi- | | |
| | | events: | ometry | | |
| | | loss of | | | |
| | | 1.65% up | | | |
| | | to 100° C. | | | |
| | | and loss of | | | |
| | | 2.1% prior | | | |
| | | to main | | | |
| | | melt | | | |
| hemi | FIG. | DSC | ¹H NMR | 7 | Off-white |
| naphthalene- | 42 | events: | confirms | | solid |
| 1,5- | bottom | small | salt | | |
| disulfonate | trace | exotherm | formation | | |
| (XRPD | | at 208° C., | and hemi- | | |
| pattern A) | | large | stoichi- | | |
| | | endotherm | ometry | | |
| | | at 262° C. | | | |
| | | TGA | | | |
| | | events: | | | |
| | | loss of | | | |
| | | 1.4% up to | | | |
| | | 100° C. and | | | |
| | | loss of | | | |
| | | 5.3% prior | | | |
| | | to main | | | |
| | | melt | | | |
| Hemi- | FIG. | DSC | ¹H NMR | 1 | Off-white |
| fumarate | 43 | events: | confirms | | solid |
| (XRPD | 2nd | Endotherms | salt | | |
| pattern A) | from | at 172 | formation | | |
| | top | and 217° C. | and hemi- | | |
| | trace | TGA | stoichi- | | |
| | | events: | ometry | | |
| | | Loss of | | | |
| | | 7.2% prior | | | |
| | | to | | | |
| | | endotherms | | | |
| | | and then | | | |
| | | loss of | | | |
| | | 4.9% over | | | |
| | | second | | | |
| | | endotherm | | | |
| Hemi- | FIG. | DSC | ¹H NMR | 2 | Off-white |
| fumarate | 43 | events: | confirms | | solid |
| (XRPD | 2nd | small | salt | | |
| pattern B) | from | endotherm | formation | | |
| | bottom | 167° C., | and hemi- | | |
| | trace | broad | stoichi- | | |
| | | endotherm | ometry | | |
| | | 223° C. | | | |
| | | TGA | | | |
| | | events: | | | |
| | | Loss of | | | |
| | | 4.5% up to | | | |
| | | 100° C. then | | | |
| | | 2.7% loss | | | |
| | | at 167° C. | | | |
| Hemi- | FIG. | DSC | ¹H NMR | 3 | Off-white |
| fumarate | 43 | events: | confirms | | solid |
| (XRPD | bottom | Single | salt | | |
| pattern C) | trace | endotherm | formation | | |
| | | at 182° C. | and hemi- | | |
| | | TGA | stoichi- | | |
| | | events: | ometry | | |
| | | 2.7% loss | | | |
| | | up to | | | |
| | | 100° C. | | | |

### Example 3A

### Determination of the Solubility of the Salts in Water

5-[[5-[4-(4-Fluoro-1-methyl-4-piperidyl)-2-methoxy-phenyl]-1H-pyrazol-3-yl]amino]pyrazine-2-carbonitrile free base and selected salts (30 mg), were weighed out into crystallisation tubes, water for injection (WFI) (1 mL) was charged and the samples left to equilibrate (25° C.) over 24 hours. The solids were isolated via vacuum filtration and the filtrates used to assess solubility using HPLC (HPLC Method 1).

| Salt | Aqueous solubility mg/mL 24 h |
|---|---|
| tosylate pattern C | 1.55 |
| maleate pattern B | 1.4 |
| bis-maleate pattern B | 0.15 |
| sulphate pattern D | 1.6 |
| besylate pattern B | 1.18 |
| bis-besylate pattern B | 0.06 |
| Free base | 0.39 |
| malonate pattern B | 7.41 |
| oxalate pattern A | 3.16 |
| hydrochloride pattern C | 10.37 |

### Conclusions

A large number of crystalline salts were identified but the majority of the salts showed a tendency for hydration/solvation and had complex thermal profiles. The known hydrochloric acid salt (disclosed in Example 64, Method L in International patent application WO 2015/20390) exhibits polymorphism and poor thermal profiles indicative of hydration/solvation and is therefore not considered to be a preferred candidate for the preparation of solid formulations.

The tosylate, maleate, besylate, malonate and oxalate all show improved solubility over the free base but the oxalate salt has a low degree of crystallinity and was therefore not considered for further development. The bis salts disproportionate in water and were therefore also not considered as candidates for further development.

### Example 3B

### Determination of the Solubility of the Salts in Biorelevant Media

### Experimental

The free base and selected salts of 5-[[5-[4-(4-fluoro-1-methyl-4-piperidyl)-2-methoxy-phenyl]-1H-pyrazol-3-yl]amino]pyrazine-2-carbonitrile (30 mg) were weighed into crystallisation tubes. Biorelevant media (DI H₂O, FeSSIF, FaSSIF and FaSSGF) (2 mL) was charged. Samples were left to equilibrate (25° C.) over 24 h. Solubility measurements were taken using HPLC (see Method 1 above).

| | FeSSIF | FaSSIF | FaSSGF |
|---|---|---|---|
| Salt form | (pH 5.14) | (pH 6.71) | (pH 1.37) |
| free base | 0.6 | 0.04 | 2.93 |
| Maleate pattern B | 0.57 | 0.07 | 3.97 |
| Besylate pattern B | 0.57 | 0.04 | 0.55 |
| Tosylate pattern C | 0.61 | 0.04 | 0.32 |
| Hydrochloride pattern C | 0.61 | 0.15 | 8.39 |
| Malonate pattern B | 0.27 | 0 | 3.24 |

FaSSIF: Fasted State Simulated Intestinal Fluid
FeSSIF: Fed State Simulated Intestinal Fluid
FaSSGF: Fasted State Simulated Gastric Fluid

The biorelevant solubility assessment of the freebase and selected salts shows overall poor solubilities of less than 1 mg/mL. As a general trend, increasing solubility was observed for the salts from FaSSIF to FeSSIF then FaSSGF. The maleate and malonate show improved solubility over the free base in FaSSGF. Most salts exhibited a similar solubility in FeSSIF and FaSSIF, but differences can be observed in the gastric fluid with the maleate salt.

The salts and free base across the biorelevant range are similar in terms of performance under these test conditions. However, maleate offers promise when the transition from gut to intestine is considered along with overall solid form performance.

The hydrochloride salt whilst soluble is polymorphic with complex thermal profiles (indicative of hydration and solvation).

### Two-Week Stability

### Protocol

Experimental: Maleate salt pattern B (30 mg) was placed into separate 15 mL type I glass vials. To these vials, a HDPE plastic cap was loosely attached to allow the ingress of moisture. The vials were then placed into ICH rated stability cabinets at 25° C./60% RH and 40° C./75% RH and in cold storage at 2-8° C. Following 2 weeks of storage these samples were removed from the stability cabinets and cold storage and the chemical purity assessed by HPLC (method 2). The relevant data was collected using a wavelength of 302 nm. The samples were prepared in MeCN:water (1:1).

The maleate salt pattern B is stable at the following conditions for two-weeks: 25° C./60% RH, 40° C./75% RH and 2-8° C.

| | | | T = 2 weeks | T = 2 weeks |
|---|---|---|---|---|
| | | T = 2 weeks | 25° C./ | 40° C./ |
| Timepoint/storage | T = 0 | 2-8° C. | 60% RH | 75% RH |
| HPLC purity | 96.85 | 96.85 | 96.99 | 96.98 |
| (HPLC method 2) | | | | |

The four best salts were the maleate, tosylate, besylate and malonate salts. Of these, the maleate salt demonstrated the best properties. Selected crystalline forms of these salts are described in more detail below.

### X-Ray Powder Diffraction Studies

### Maleate Pattern B

The XRPD spectrum for maleate Pattern B is shown in FIG. 25 and the thermal data are shown in FIG. 26. The XRPD peaks for Pattern B are set out in the table below.

| Pos. [°2Th.] | Height [cts] | FWHM [°2Th.] | d-spacing [A) | Rel. Int. [%] |
|---|---|---|---|---|
| 5.6284 | 187.86 | 0.6140 | 15.70212 | 3.50 |
| 6.9241 | 4611.31 | 0.0768 | 12.76650 | 86.00 |
| 9.3940 | 2052.07 | 0.0768 | 9.41471 | 38.27 |
| 11.7686 | 4879.50 | 0.1023 | 7.51989 | 91.00 |
| 12.4667 | 224.33 | 0.1023 | 7.10028 | 4.18 |
| 13.1479 | 452.73 | 0.1023 | 6.73392 | 8.44 |
| 13.4712 | 887.44 | 0.1023 | 6.57305 | 16.55 |
| 14.0901 | 1108.81 | 0.1023 | 6.28568 | 20.68 |
| 14.4106 | 451.78 | 0.1279 | 6.14658 | 8.43 |
| 15.6116 | 2210.87 | 0.1279 | 5.67633 | 41.23 |
| 15.8143 | 2118.49 | 0.1023 | 5.60405 | 39.51 |
| 16.1313 | 475.62 | 0.1279 | 5.49462 | 8.87 |
| 16.5029 | 216.63 | 0.1023 | 5.37172 | 4.04 |
| 17.2705 | 682.34 | 0.1535 | 5.13467 | 12.73 |
| 17.6733 | 2969.71 | 0.1023 | 5.01853 | 55.38 |
| 17.9199 | 4342.69 | 0.1279 | 4.95003 | 80.99 |
| 18.2543 | 1106.15 | 0.1279 | 4.86010 | 20.63 |
| 18.6741 | 1986.10 | 0.1279 | 4.75178 | 37.04 |
| 19.1654 | 995.76 | 0.1279 | 4.63105 | 18.57 |
| 19.9459 | 98.11 | 0.1535 | 4.45156 | 1.83 |
| 20.7957 | 450.57 | 0.1535 | 4.27154 | 8.40 |
| 21.8726 | 1930.42 | 0.1535 | 4.06361 | 36.00 |
| 22.3012 | 1015.57 | 0.1279 | 3.98647 | 18.94 |
| 22.7998 | 134.49 | 0.1535 | 3.90041 | 2.51 |
| 23.6624 | 344.56 | 0.1279 | 3.76014 | 6.43 |
| 23.9351 | 633.64 | 0.1279 | 3.71791 | 11.82 |
| 24.8019 | 214.73 | 0.1279 | 3.58991 | 4.00 |
| 25.0411 | 212.35 | 0.1023 | 3.55615 | 3.96 |
| 25.7311 | 769.93 | 0.1279 | 3.46234 | 14.36 |
| 26.1114 | 649.57 | 0.0768 | 3.41276 | 12.11 |
| 26.4273 | 5361.98 | 0.1791 | 3.37269 | 100.00 |
| 26.8310 | 2088.44 | 0.1535 | 3.32284 | 38.95 |
| 27.3094 | 785.65 | 0.1535 | 3.26572 | 14.65 |
| 27.7550 | 1972.89 | 0.1791 | 3.21429 | 36.79 |
| 29.0081 | 581.05 | 0.2047 | 3.07822 | 10.84 |
| 29.6415 | 151.62 | 0.1791 | 3.01387 | 2.83 |
| 30.5909 | 242.30 | 0.1791 | 2.92247 | 4.52 |
| 31.7416 | 419.84 | 0.1535 | 2.81910 | 7.83 |
| 32.3802 | 138.18 | 0.2558 | 2.76495 | 2.58 |
| 33.4454 | 85.94 | 0.1535 | 2.67928 | 1.60 |
| 34.6245 | 22.98 | 0.1535 | 2.59069 | 0.43 |

### Maleate Pattern A

The XRPD spectrum for maleate Pattern A is shown in FIG. 27 and the thermal data are shown in FIG. 28. The XRPD peaks for Pattern A are set out in the table below.

| Pos. [°2Th.] | Height [cts] | FWHM [°2Th.] | d-spacing [Å] | Rel. Int. [%] |
|---|---|---|---|---|
| 5.2880 | 79.08 | 0.5117 | 16.71232 | 8.82 |
| 6.5960 | 896.14 | 0.1791 | 13.40077 | 100.00 |
| 9.2386 | 337.42 | 0.1535 | 9.57269 | 37.65 |
| 11.1317 | 489.61 | 0.1279 | 7.94868 | 54.64 |
| 11.5356 | 253.70 | 0.1023 | 7.67125 | 28.31 |
| 14.2833 | 284.59 | 0.1535 | 6.20110 | 31.76 |
| 15.6422 | 179.45 | 0.1535 | 5.66529 | 20.02 |
| 16.0500 | 171.59 | 0.1023 | 5.52227 | 19.15 |
| 16.9462 | 219.43 | 0.1535 | 5.23218 | 24.49 |
| 17.3430 | 619.49 | 0.1279 | 5.11335 | 69.13 |
| 18.5483 | 272.31 | 0.2047 | 4.78371 | 30.39 |
| 18.9422 | 154.41 | 0.0553 | 4.68513 | 17.23 |
| 19.7251 | 68.05 | 0.0900 | 4.50089 | 7.59 |
| 20.5367 | 212.24 | 0.1279 | 4.32482 | 23.68 |
| 21.6425 | 113.90 | 0.3070 | 4.10628 | 12.71 |
| 22.0699 | 126.37 | 0.1535 | 4.02772 | 14.10 |
| 22.8840 | 84.48 | 0.2047 | 3.88625 | 9.43 |
| 25.5206 | 110.36 | 0.2047 | 3.49041 | 12.31 |
| 25.8745 | 263.80 | 0.1092 | 3.44347 | 29.44 |
| 26.5069 | 385.90 | 0.1535 | 3.36274 | 43.06 |
| 27.6811 | 75.78 | 0.0900 | 3.22270 | 8.46 |
| 28.6708 | 111.36 | 0.2047 | 3.11367 | 12.43 |

### Maleate Pattern C

The XRPD spectrum for maleate Pattern C is shown in FIG. 29 and the thermal data are shown in FIG. 30. The XRPD peaks for Pattern C are set out in the table below

| Pos. [°2Th.] | Height [cts] | FWHM [°2Th.] | d-spacing [Å] | Rel. Int. [%] |
|---|---|---|---|---|
| 5.3327 | 83.03 | 0.4093 | 16.57231 | 5.52 |
| 6.6739 | 1504.73 | 0.1791 | 13.24447 | 100.00 |
| 9.1925 | 817.35 | 0.1279 | 9.62061 | 54.32 |
| 11.1462 | 422.05 | 0.1279 | 7.93834 | 28.05 |
| 11.5411 | 935.39 | 0.1535 | 7.66761 | 62.16 |
| 13.2677 | 195.85 | 0.1535 | 6.67338 | 13.02 |
| 13.8736 | 293.30 | 0.1279 | 6.38327 | 19.49 |
| 14.2687 | 541.48 | 0.1791 | 6.20742 | 35.99 |
| 15.6379 | 768.88 | 0.1279 | 5.66687 | 51.10 |
| 15.9603 | 359.30 | 0.1279 | 5.55308 | 23.88 |
| 16.9613 | 395.42 | 0.1791 | 5.22755 | 26.28 |
| 17.3625 | 1072.27 | 0.1279 | 5.10766 | 71.26 |
| 17.7298 | 1113.95 | 0.1279 | 5.00266 | 74.03 |
| 18.5399 | 668.08 | 0.2047 | 4.78585 | 44.40 |
| 20.5008 | 285.44 | 0.2558 | 4.33231 | 18.97 |
| 21.6905 | 539.92 | 0.1535 | 4.09730 | 35.88 |
| 22.1596 | 358.58 | 0.2047 | 4.01163 | 23.83 |
| 23.7465 | 154.55 | 0.1535 | 3.74701 | 10.27 |
| 25.5692 | 386.89 | 0.2047 | 3.48389 | 25.71 |
| 26.2698 | 1327.41 | 0.1535 | 3.39254 | 88.22 |
| 27.5980 | 420.43 | 0.1791 | 3.23222 | 27.94 |
| 28.8551 | 190.05 | 0.6140 | 3.09420 | 12.63 |
| 30.4932 | 57.31 | 0.3070 | 2.93161 | 3.81 |
| 31.5105 | 77.47 | 0.6140 | 2.83925 | 5.15 |

### Malonate Pattern B

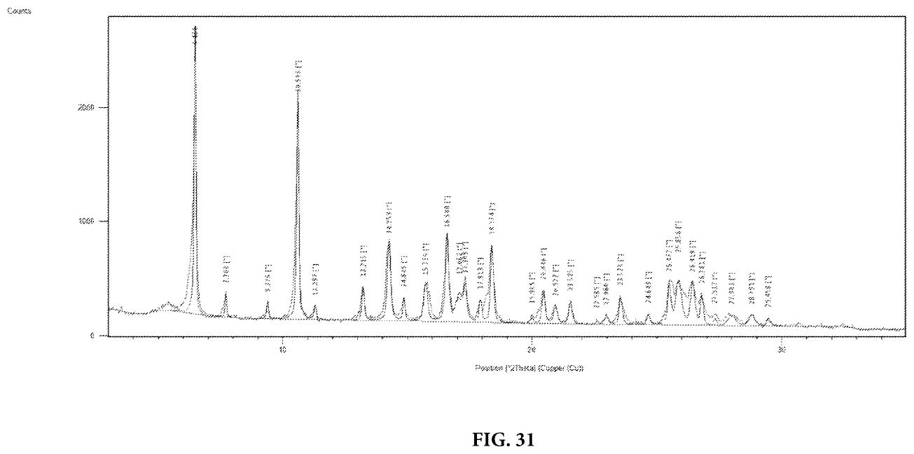

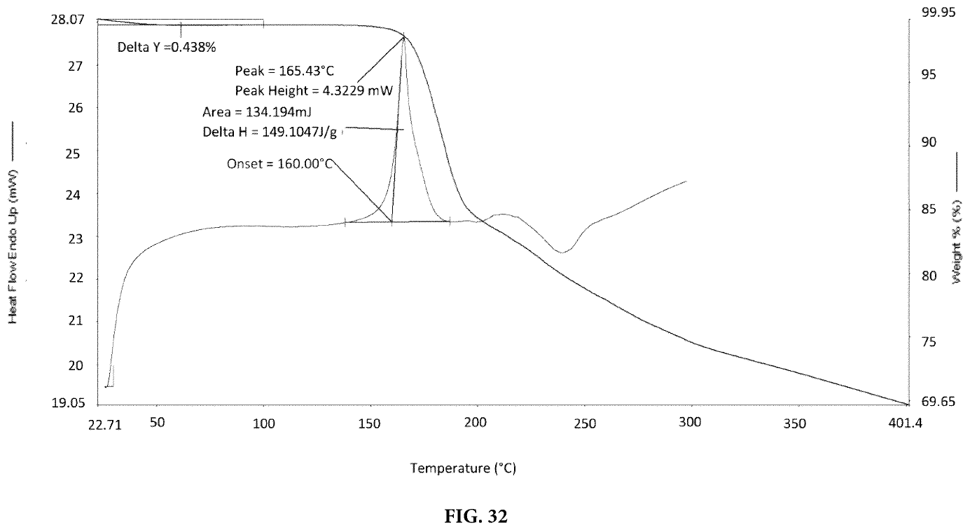

The XRPD spectrum for malonate Pattern B is shown in FIG. 31 and the DSC and TGA traces are shown in FIG. 32. The XRPD peaks are listed in the table below.

| Pos. [°2Th.] | Height [cts] | FWHM [°2Th.] | d-spacing [Å] | Rel. Int. [%] |
|---|---|---|---|---|
| 6.4684 | 2427.30 | 0.1023 | 13.66496 | 100.00 |
| 7.7000 | 204.63 | 0.0768 | 11.48178 | 8.43 |
| 9.3757 | 142.91 | 0.1023 | 9.43303 | 5.89 |
| 10.5956 | 1899.35 | 0.1023 | 8.34958 | 78.25 |
| 11.2972 | 114.47 | 0.1023 | 7.83254 | 4.72 |
| 13.2150 | 287.40 | 0.1023 | 6.69989 | 11.84 |
| 14.2527 | 666.30 | 0.1535 | 6.21434 | 27.45 |
| 14.8453 | 202.81 | 0.1023 | 5.96757 | 8.36 |
| 15.7156 | 313.92 | 0.1791 | 5.63902 | 12.93 |
| 16.5805 | 729.51 | 0.1535 | 5.34677 | 30.05 |
| 17.0621 | 320.41 | 0.3048 | 5.19691 | 13.20 |
| 17.3089 | 335.35 | 0.1279 | 5.12335 | 13.82 |
| 17.9127 | 194.99 | 0.1023 | 4.95199 | 8.03 |
| 18.3739 | 675.01 | 0.1279 | 4.82872 | 27.81 |
| 19.9851 | 74.62 | 0.0900 | 4.44292 | 3.07 |
| 20.4457 | 290.41 | 0.1279 | 4.34387 | 11.96 |
| 20.9222 | 146.01 | 0.1535 | 4.24600 | 6.02 |
| 21.5253 | 193.94 | 0.1535 | 4.12838 | 7.99 |
| 22.5851 | 43.74 | 0.0900 | 3.93699 | 1.80 |
| 22.9598 | 73.53 | 0.1535 | 3.87358 | 3.03 |
| 23.5282 | 244.44 | 0.1279 | 3.78128 | 10.07 |
| 24.6490 | 93.06 | 0.1279 | 3.61183 | 3.83 |
| 25.4768 | 373.34 | 0.1279 | 3.49631 | 15.38 |
| 25.8559 | 537.48 | 0.2555 | 3.44591 | 22.14 |
| 26.4188 | 372.48 | 0.1791 | 3.37375 | 15.35 |
| 26.7811 | 276.03 | 0.1023 | 3.32892 | 11.37 |
| 27.3171 | 95.19 | 0.0900 | 3.26481 | 3.92 |
| 27.9810 | 90.05 | 0.3070 | 3.18884 | 3.71 |
| 28.7910 | 91.75 | 0.2558 | 3.10094 | 3.78 |
| 29.4577 | 68.42 | 0.1535 | 3.03227 | 2.82 |

### Tosylate Pattern A

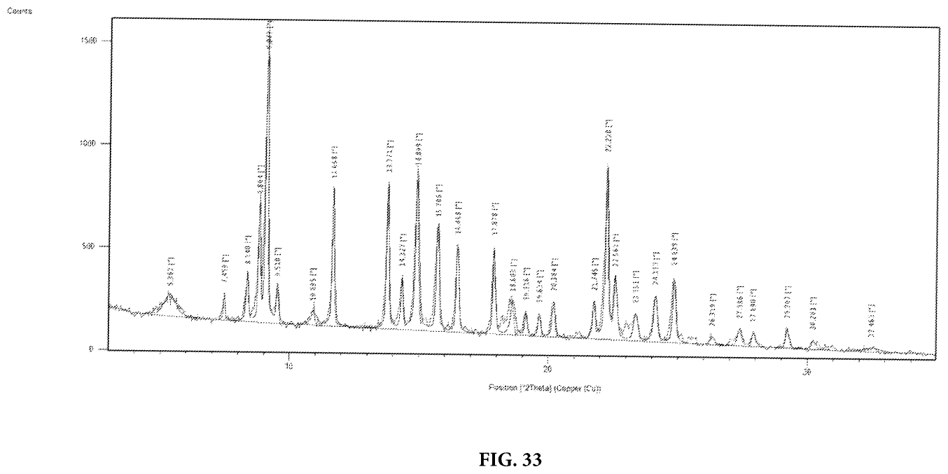

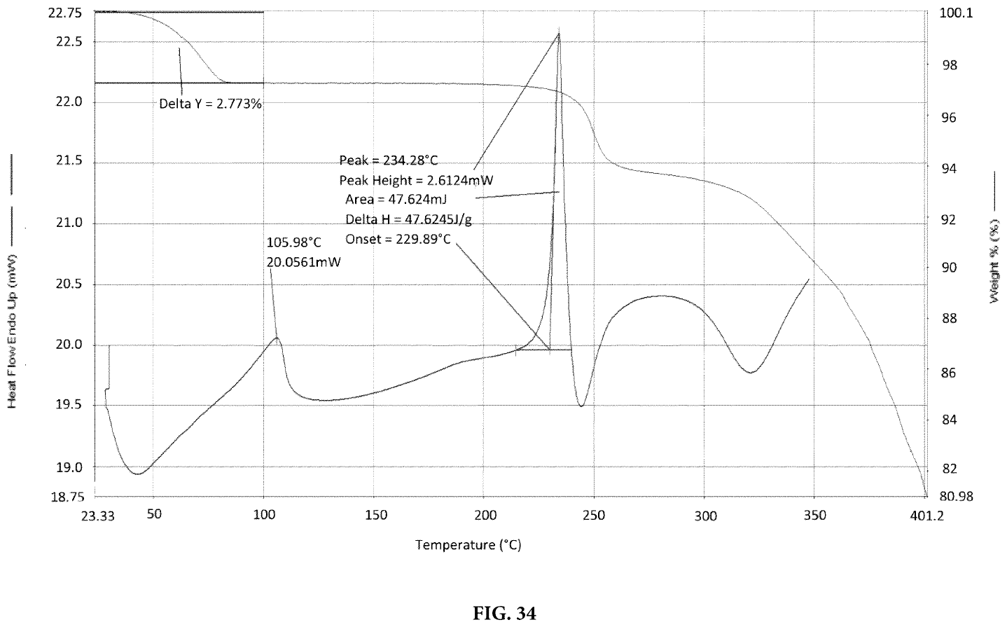

The XRPD spectrum for tosylate Pattern A is shown in FIG. 33 and the TGA and DSC traces are shown in FIG. 34. The XRPD peaks are listed in the table below.

| Pos. [°2Th.] | Height [cts] | FWHM [°2Th.] | d-spacing [Å] | Rel. Int. [%] |
|---|---|---|---|---|
| 5.3623 | 94.92 | 0.5117 | 16.48094 | 6.90 |
| 7.4588 | 129.28 | 0.0768 | 11.85248 | 9.39 |
| 8.3404 | 226.46 | 0.1023 | 10.60158 | 16.45 |
| 8.8036 | 572.85 | 0.0768 | 10.04469 | 41.61 |
| 9.0769 | 1376.59 | 0.1023 | 9.74292 | 100.00 |
| 9.5099 | 177.22 | 0.1023 | 9.30022 | 12.87 |
| 10.8948 | 62.90 | 0.3070 | 8.12092 | 4.57 |
| 11.6679 | 660.29 | 0.1023 | 7.58452 | 47.97 |
| 13.7713 | 704.95 | 0.1023 | 6.43045 | 51.21 |
| 14.3266 | 249.95 | 0.1023 | 6.18246 | 18.16 |
| 14.8992 | 743.71 | 0.1535 | 5.94609 | 54.03 |
| 15.7056 | 500.19 | 0.1279 | 5.64257 | 36.34 |
| 16.4685 | 421.65 | 0.1023 | 5.38288 | 30.63 |
| 17.8776 | 425.16 | 0.1023 | 4.96165 | 30.89 |
| 18.6033 | 156.43 | 0.2047 | 4.76969 | 11.36 |
| 19.1160 | 117.00 | 0.1023 | 4.64292 | 8.50 |
| 19.6343 | 107.84 | 0.1023 | 4.52152 | 7.83 |
| 20.1841 | 168.51 | 0.1279 | 4.39956 | 12.24 |
| 21.7448 | 180.09 | 0.1279 | 4.08720 | 13.08 |
| 22.2203 | 854.48 | 0.1279 | 4.00080 | 62.07 |
| 22.5619 | 302.16 | 0.1279 | 3.94098 | 21.95 |
| 23.3509 | 126.53 | 0.1791 | 3.80959 | 9.19 |
| 24.1135 | 221.69 | 0.1535 | 3.69081 | 16.10 |
| 24.8389 | 320.64 | 0.1279 | 3.58463 | 23.29 |
| 26.3188 | 35.80 | 0.2047 | 3.38634 | 2.60 |
| 27.3858 | 77.29 | 0.2047 | 3.25678 | 5.61 |
| 27.8900 | 66.59 | 0.1535 | 3.19904 | 4.84 |
| 29.2068 | 88.90 | 0.1535 | 3.05773 | 6.46 |
| 30.2006 | 39.65 | 0.2047 | 2.95934 | 2.88 |
| 32.4628 | 17.05 | 0.8187 | 2.75810 | 1.24 |

### Besylate Pattern C

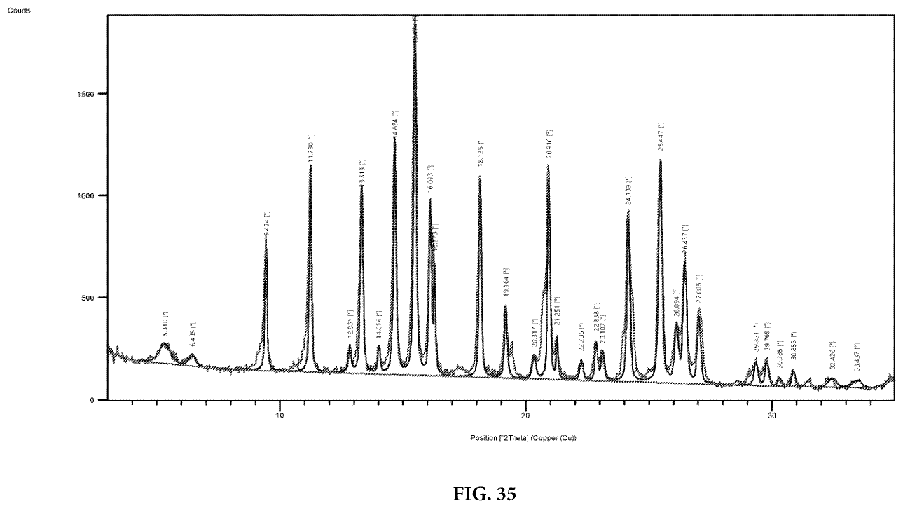

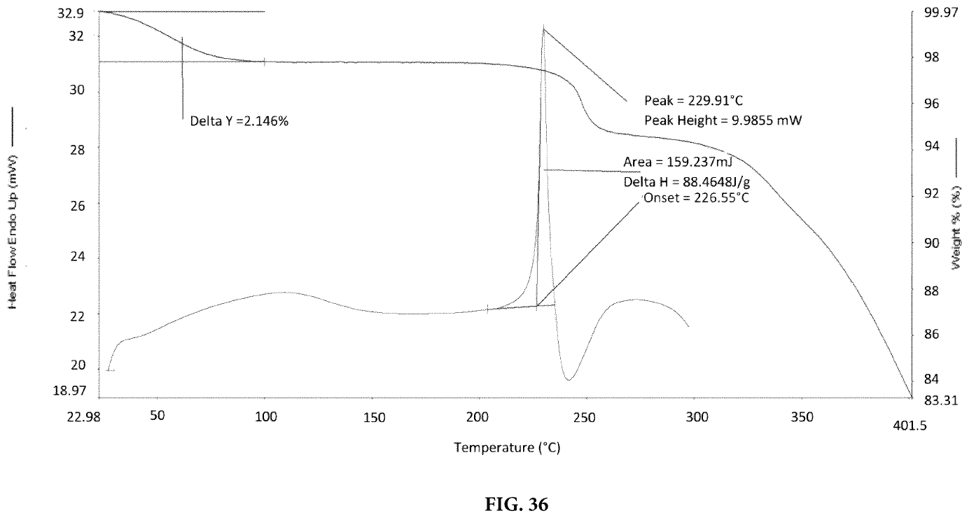

The XRPD spectrum for besylate Pattern C is shown in FIG. 35 and the TGA and DSC traces are shown in FIG. 36. The XRPD peaks are listed in the table below.

| Pos. [°2Th.] | Height [cts] | FWHM [°2Th.] | d-spacing [Å] | Rel. Int. [%] |
|---|---|---|---|---|
| 5.3099 | 88.50 | 0.5117 | 16.64342 | 5.28 |
| 6.4346 | 49.98 | 0.3070 | 13.73667 | 2.98 |
| 9.4241 | 614.80 | 0.1023 | 9.38471 | 36.66 |
| 11.2303 | 980.94 | 0.1023 | 7.87909 | 58.49 |
| 12.8313 | 123.05 | 0.1279 | 6.89936 | 7.34 |
| 13.3125 | 865.00 | 0.1279 | 6.65103 | 51.57 |
| 14.0143 | 126.51 | 0.1023 | 6.31949 | 7.54 |
| 14.6542 | 1108.95 | 0.1279 | 6.04495 | 66.12 |
| 15.4744 | 1677.21 | 0.1279 | 5.72635 | 100.00 |
| 16.0933 | 847.34 | 0.1023 | 5.50749 | 50.52 |
| 16.2732 | 561.39 | 0.0768 | 5.44701 | 33.47 |
| 18.1250 | 984.95 | 0.1023 | 4.89448 | 58.73 |
| 19.1636 | 351.15 | 0.1279 | 4.63148 | 20.94 |
| 20.3168 | 109.03 | 0.1535 | 4.37113 | 6.50 |
| 20.9164 | 1037.05 | 0.1023 | 4.24716 | 61.83 |
| 21.2514 | 219.63 | 0.0768 | 4.18097 | 13.10 |
| 22.2353 | 95.89 | 0.1535 | 3.99814 | 5.72 |
| 22.8379 | 195.27 | 0.1023 | 3.89399 | 11.64 |
| 23.1023 | 139.88 | 0.1535 | 3.85001 | 8.34 |
| 24.1395 | 821.07 | 0.1279 | 3.68689 | 48.95 |
| 25.4472 | 1090.62 | 0.1535 | 3.50032 | 65.03 |
| 26.0941 | 280.14 | 0.1535 | 3.41498 | 16.70 |
| 26.4366 | 592.68 | 0.1535 | 3.37151 | 35.34 |
| 27.0051 | 350.71 | 0.1279 | 3.30181 | 20.91 |
| 29.3206 | 118.40 | 0.1535 | 3.04613 | 7.06 |
| 29.7647 | 126.30 | 0.1279 | 3.00168 | 7.53 |
| 30.2846 | 37.92 | 0.1535 | 2.95133 | 2.26 |
| 30.8533 | 82.31 | 0.1279 | 2.89821 | 4.91 |
| 32.4258 | 40.50 | 0.3582 | 2.76116 | 2.41 |
| 33.4370 | 33.42 | 0.4093 | 2.67994 | 1.99 |

### Gravimetric Vapour Sorption Studies

GVS data obtained using the protocol described above are set out below for certain of the crystalline forms of the salts.

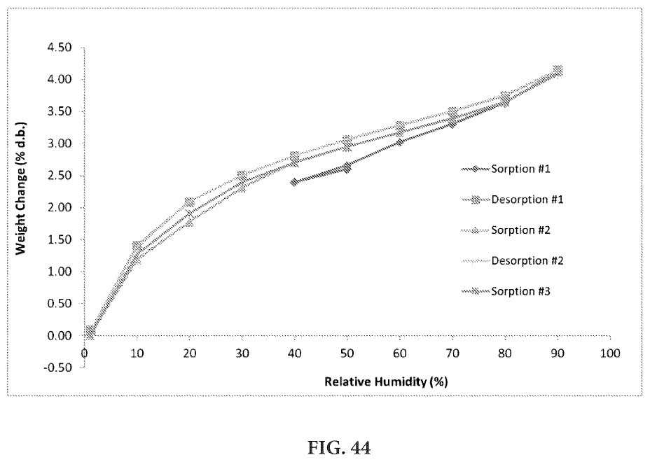

### Maleate Pattern A—see FIG. 44

During the initial sorption cycle the solid gained 1.5 wt % from 50% RH to 90% RH. During the subsequent desorption cycle the solid lost 4% of water down to 0% RH. This increased to 4 wt % at 90% RH on the following sorption cycles. The GVS profile confirms that this water uptake is reversible as relative humidity decreases with only minor hysteresis indicated.

A new pattern was isolated at 0 and 90% RH and was named B. Pattern B is closely related to pattern A.

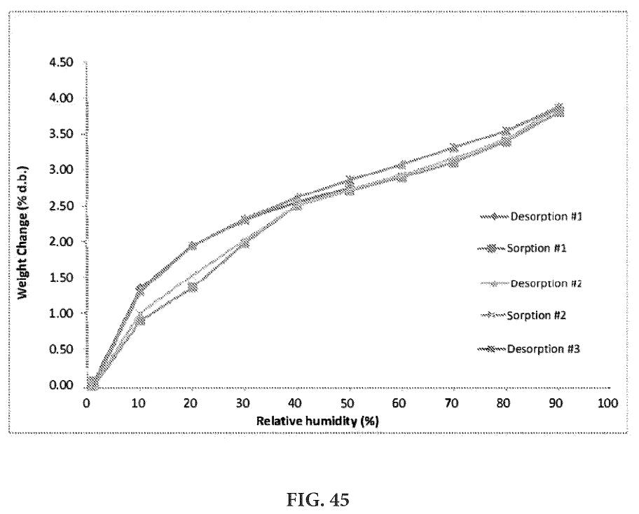

### Maleate Pattern B—see FIG. 45

During the initial desorption cycle the solid loses 2.5 wt % from 50% RH to 0% RH. During the subsequent sorption cycle the solid gains 4% of water up to 90% RH with a sharp increase noted between 0% RH and 40% RH. 0% RH converts to pattern A, 90% RH no change. This data suggests interconversion between crystalline versions is linked to hydration.

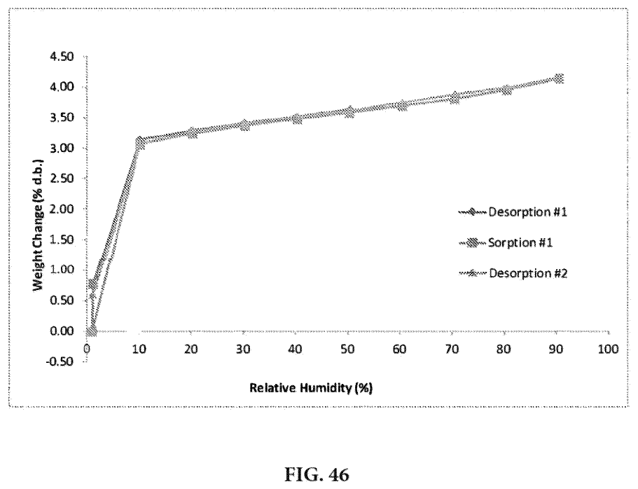

### Tosylate Pattern A—FIG. 46

During the initial desorption cycle the solid loses 3.5 wt % from 50% RH to 0% RH with a steady decrease of 0.5 wt % from 50% RH to 10% RH and then a sharp decrease of ˜3 wt % from 10% RH to 0% RH. During the subsequent sorption cycle the solid sharply gains ˜3% of water up to 10% RH with a steady increase of ˜1% from 10% RH to 90% RH. A 3% water content equates to a mono hydrate of the tosylate salt. No form change at 0% RH and 90% RH, suggests channel hydrate, reversible and stable across ambient range.

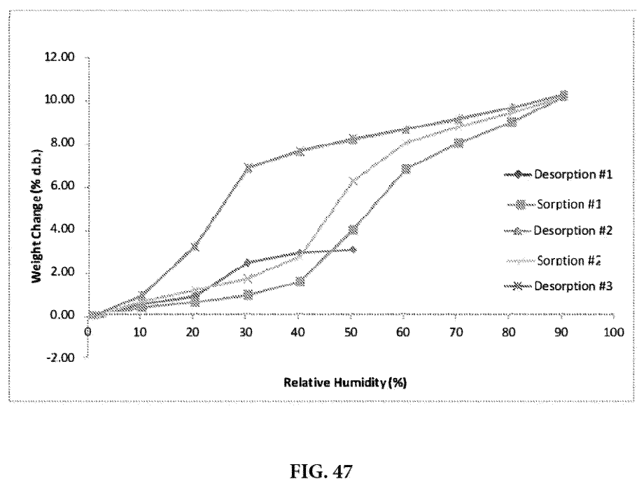

### Besylate Pattern A—See FIG. 47

During the initial desorption cycle, the solid loses 2.5 wt % from 50% RH to 0% RH. During the subsequent sorption cycle the solid gains 10% of water up to 90% RH with a sharp increase of 6% from 40% RH to 60% RH. The following desorption cycle shows a steady decrease of ˜3 wt % from 90% RH to 30% RH and then sharp decrease to 0% wt % from 30% to 0% RH. The theoretical amount of water required for a formal mono hydrate of the besylate salt is 3.6%. Therefore, this version of the salt is hydrating to a trihydrate.

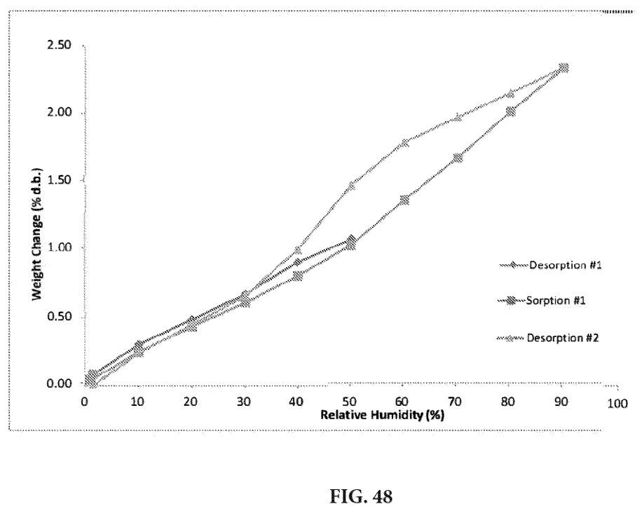

### Besylate Pattern B—See FIG. 48

During the initial desorption cycle the solid loses 1 wt % from 50% RH to 0% RH. During the subsequent sorption cycle the solid gains ˜2.25% of water up to 90% RH with a steady increase noted. This Pattern B version of the besylate shows a more positive GVS profile to that of the Pattern A.

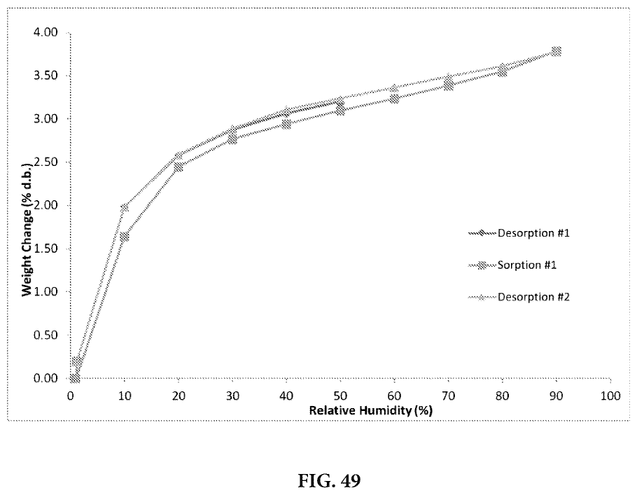

### Besylate Pattern C—See FIG. 49

During the initial desorption cycle the solid loses ˜3 wt % from 50% RH to 0% RH. During the subsequent sorption cycle the solid gains 3.75% of water up to 90% RH with a sharp increase noted between 0% RH and 20% RH of ˜2.5 wt %.

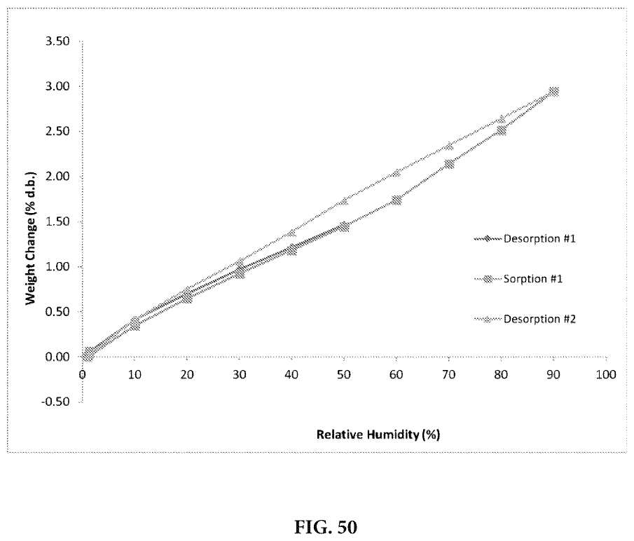

### Naphthalene-2-Sulfonate Pattern A—See FIG. 50

During the initial desorption cycle the solid loses 1 wt % from 50% RH to 0% RH. During the subsequent sorption cycle the solid gains ˜2.25% of water up to 90% RH with a steady increase noted. XRPD analysis showed that no change in the crystallinity of the solid occurred at extremes of humidity.

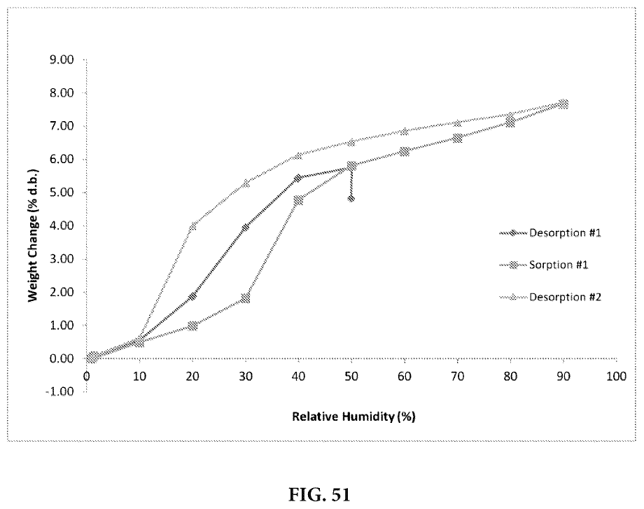

### Malonate Pattern B—See FIG. 51

The GVS profile shows the material does lose 5 wt % on the initial desorption step to 0% RH. The material is therefore believed to be hygroscopic and has hydrated to a non-stoichiometric level in ambient conditions. During the subsequent sorption cycle the solid gains 7.5% of water up to 90% RH with a sharp increase noted between 30% RH and 40% RH of ˜3 wt %. This water uptake is reversible with the water absorbed lost as relative humidity decreases. The theoretical amount of water required for a formal mono hydrate of the malonate salt is 3.4% so the salt is hydrating up to a dihydrate level in extremes of moisture.

### Example 4

Further Investigations into the Maleate Salts

Five crystalline patterns were identified for the maleate salt and these are labelled Pattern A, Pattern B, Pattern C, Pattern D and Pattern E. Characterising data for Patterns A, B and C are described above and characterising data for Pattern D and Pattern E are described below.

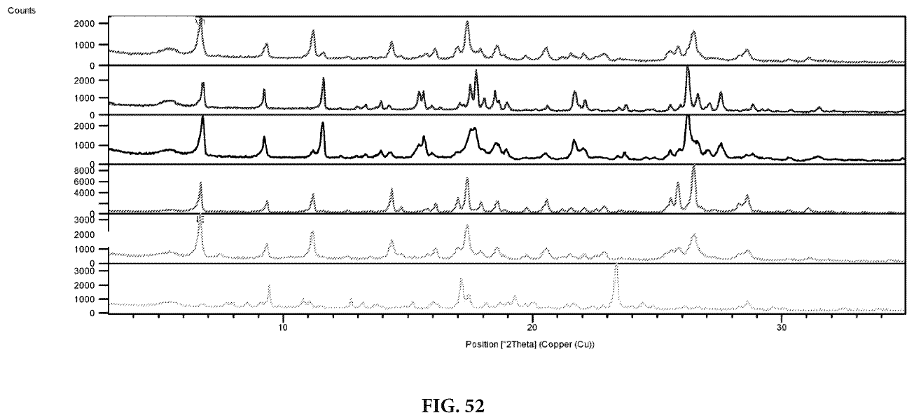

A comparison of the XRPD spectra of the five crystalline patterns and a mixture of the A/B patterns is shown in FIG. 52.

Patterns A, B, C and D appear to be variants having differing degrees of hydration. Pattern A has been found to be difficult to isolate as it turns to a mixture of A and B as soon as any moisture is absorbed. Pattern B is a relatively stable hydrate whereas Pattern C is believed to be a non-stoichiometric hydrate. Pattern D is also believed to be a non-stoichiometric hydrate and is similar to Pattern C. Pattern E is an N-methylpyrrolidone (NMP) solvate.

### 4A. Preparation of Maleate Salt Pattern a Via a Pattern A/B Mixture Followed by Thermal Cycling

The free base of 5-[[5-[4-(4-fluoro-1-methyl-4-piperidyl)-2-methoxy-phenyl]-1H-pyrazol-3-yl]amino]pyrazine-2-carbonitrile (4.9756 g) was charged into a round bottom flask and charged with THF (204 mL, 41 vols). The mixture was heated to 60° C. The solution was then charged with maleic acid (1.48 g, 1 eq) as a solution in THF (2 vols). The mixture was left to equilibrate at 60° C. for 1 hour and then left to cool to 20° C. overnight, giving a beige suspension. The solid was isolated by filtration in vacuo and washed with THF. The solid was dried at 40° C. in vacuo for 20 hour to afford maleate salt (SSA203).

A portion of the resulting solid (SSA203) was weighed into a crystallisation tube (50 mg) and charged with methyl isobutyl ketone (5 vols). The mixture was equilibrated at room temperature for 18 hours to afford pattern A/B mixture. A thermal cycle was performed by heating to 150° C. to afford maleate salt pattern A.

### 4B. Preparation of Maleate Salt Pattern a by a High Boiling Non-Aqueous Solvent Method

SSA203 (from Example 4A) was weighed into crystallisation tubes (60 mg/tube) and charged with the appropriate high boiling point solvent (10 vols). The mixtures were equilibrated at RT for ca. 30 mins, heated to 95° C. and equilibrated for 4 hours and then left to naturally cool to RT over 70 hours. The mixtures were then heated to 95° C. again, equilibrated for 4 hours and left to cool to RT over 3 hours. The solids were isolated and dried at 45° C. for 18 hours.

The solvents and resulting maleate salt pattern are shown in the table below.

| Sample ID | Solvent (volumes) | XRPD dry solids |
|---|---|---|
| SSA243-A | Xylenes (10) | Pattern A |
| SSA243-D | n-PrOAc (10) | Pattern A |
| SSA243-F | Decalin (10) | Pattern A |
| SSA243-G | Dioxane (10) | Pattern A |
| SSA243-H | 1-BuOH (10) | Pattern A |

### 4C. Anti-Solvent Mediated Recrystallisation

5-[[5-[4-(4-fluoro-1-methyl-4-piperidyl)-2-methoxy-phenyl]-1H-pyrazol-3-yl]amino]pyrazine-2-carbonitrile maleate (SSA203 from Example 4A)) was weighed into 2 crystallisation tubes and charged with either DMSO (4 vols) or NMP (4 vols). The mixtures were heated to 60° C. The yellow solutions were then clarified into clean, pre-heated tubes at 60° C. The clarified solutions were then split into aliquots of 320 μl so that each tube would contain 80 mg of the maleate salt. The solutions were then charged with the appropriate anti-solvent in 0.5 to 1 volume aliquots with equilibrations for a minimum of 10 minutes after each addition until a hazy solution formed or until 10 volumes of anti-solvent were added. The mixtures were then left to equilibrate at 60° C. for ca. 30 minutes and then cooled to 25° C. and equilibrated for ca. 20 hours.

Those entries that remained as solutions were cooled to 0° C. and equilibrated for ca. 6 hours. The mixtures that remained as solutions at 0° C. were heated to 60° C., ca. half of the solvent was evaporated by a gentle stream of nitrogen and cooled back to ambient temperature.

The crystalline patterns isolated from the various solvent combinations are shown below.
Pattern B for the solids isolated from DMSO/water, NMP/MeCN and NMP/water
Pattern A/B mixture from NMP/BuOH
Pattern C isolated from DMSO/BuOH and DMSO/MeCN
Pattern D isolated from THF+flash evaporation
Pattern E isolated from NMP/Dioxane, NMP/n-PrOAc, NMP/Toluene, NMP/THF and NMP/EtOAc

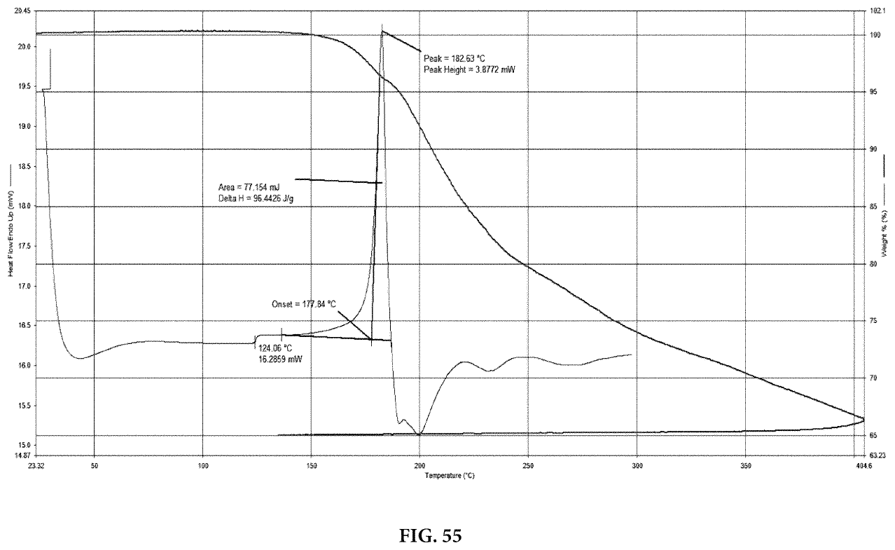

The DSC and TGA profiles of maleate salt Pattern E are shown in FIG. 55.

### 4D. Preparation of Maleate Salt Pattern D

The free base of 5-[[5-[4-(4-fluoro-1-methyl-4-piperidyl)-2-methoxy-phenyl]-1H-pyrazol-3-yl]amino]pyrazine-2-carbonitrile (4.9756 g) was charged into a round bottom flask and charged with THF (204 mL, 41 vols). The mixture was heated to 60° C. The solution was then charged with maleic acid (1.48 g, 1 eq) as a solution in of THF (2 vols). The mixture was left to equilibrate at 60° C. for 1 hour. 100 ml of the solution was clarified into a clean, pre-heated flask at 60° C., left to cool to ca. 50° C. and flash evaporated to afford maleate salt pattern D.

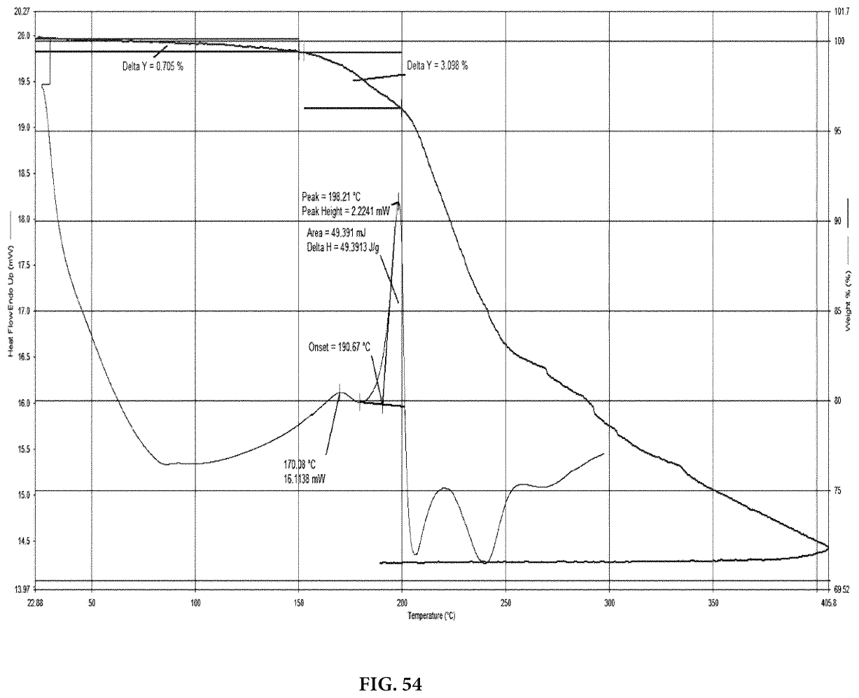

The DSC and TGA profiles of maleate salt Pattern E are shown in FIG. 54.

### 4E. Synthesis of Amorphous Maleate Salt

5-[[5-[4-(4-Fluoro-1-methyl-4-piperidyl)-2-methoxy-phenyl]-1H-pyrazol-3-yl]amino]pyrazine-2-carbonitrile maleate (583.4 mg) was dissolved in hexafluoro-2-propanol (6F-IPA, 6 vols, 1750 μL) at 30° C. The solution was clarified into a tube already charged with tert-butyl methyl ether (TBME, 6 mL) and cooled to 0° C. The mixtures were stirred at 0° C. for 15 mins and the solid was isolated by filtration in vacuo and dried at 45° C. over a period of 18 hours.

### 4F. Formation of Maleate Pattern B by Conditioning of Pattern A

Maleate salt pattern A was conditioned using a warm vacuum oven (25° C., slight vacuum bleed to provide an active flow through the oven) and a source of moisture (static, tray of deionised water) over 48 hours with continual monitoring via a multi-sample approach (XRPD samples) across the conditioning tray until all samples reported Pattern B.

### 4G. Dynamic Vapour Sorption (DVS) Analysis of Maleate Salt Pattern B

A defined amount of the maleate salt Pattern B was placed in a tared mesh stainless steel basket under ambient conditions. A full experimental cycle consisted of five scans (desorption, sorption repeat and desorption) at a constant temperature (25° C.) and 10% RH intervals over a 0-90% range (60 minutes for each humidity level). This type of extended experiment should demonstrate the ability of the sample studied to absorb moisture (or not) over a set of well-determined humidity ranges.

Post cycle, the material was isolated at 0% RH and tested for crystallinity and then held at 90% RH for a minimum of 3 hours and re-tested for changes in crystallinity.

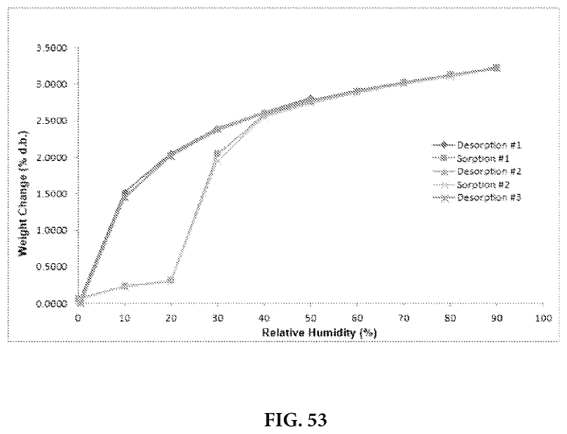

The results are shown in FIG. 53.

The solid showed ca. 2.8 wt % moisture associated before the first desorption. During the first sorption, the main increase in weight was between 20 and 30% RH (ca. 2 wt %). After 5 cycles, the material returned to 0, with no moisture associated.

XRPD analysis indicated a mixed phase at 0% RH and pattern B at 90% RH. This profile with associated hysteresis between 30-0% RH is typical of a reversible channel hydrate whose transition from anhydrate to hydrate kinetically requires time above 30% RH to equilibrate.

### Biological Activity

### Example A

### Chk-1 Kinase Inhibiting Activity

The compounds of formula (1) (5-[[5-[4-(4-fluoro-1-methyl-4-piperidyl)-2-methoxy-phenyl]-1H-pyrazol-3-yl]amino]pyrazine-2-carbonitrile) has been tested for activity against Chk-1 kinase using the materials and protocols set out below.

### Reaction Buffer:

Base Reaction buffer: 20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO
Required cofactors are added individually to each kinase reaction

### Reaction Procedure:

(i) Prepare indicated substrate in freshly prepared Base Reaction Buffer
(ii) Deliver any required cofactors to the substrate solution above
(iii) Deliver indicated kinase into the substrate solution and gently mix
(iv) Deliver compounds in DMSO into the kinase reaction mixture
(v) Deliver ³³P-ATP (specific activity 0.01 Ci/l final) into the reaction mixture to initiate the reaction.
(vi) Incubate kinase reaction for 120 minutes at room temperature
(vii) Reactions are spotted onto P81 ion exchange paper (Whatman #3698-915)
(viii) Wash filters extensively in 0.1% phosphoric acid.
(ix) Dry filters and measure counts in scintillation counter

### Kinase Information:

### CHK-1—Genbank Accession #AF016582

Recombinant full length construct, N-terminal GST tagged, purified from insect cells.

No special measures were taken to activate this kinase.
Final concentration in assay=0.5 nM
Substrate: CHKtide
Peptide sequence: [KKKVSRSGLYRSPSMPENLNRPR]
Final concentration in assay=20 μM
No additional cofactors are added to the reaction mixture

From the results obtained by following the above protocol, the IC₅₀ values against Chk-1 kinase of the compound of formula (1) has been determined as being 0.00015 μM.

### Example B

### Gemcitabine Combination Cell Assay

Exponentially growing MIA PaCa-2 (ATCC CRL-1420) cells are treated with trypsin to remove cells from the plate surface. Approximately 10,000 cells/well are plated in 96 well plates in RPMI containing 10% fetal bovine serum, 1% sodium pyruvate and 1% L-GlutaMax. Cells are allowed to adhere to the plate surface overnight. Serial half-log dilutions of Chk1 inhibitor test compounds and gemcitabine are made with a final highest concentration of 3000 nM and 100 nM, respectively. Chk1 inhibitors and gemcitabine are combined so that each concentration of Chk1 inhibitor is added to each concentration of gemcitabine. Each drug is also tested as a single agent. Drugs are added to adherent cells (in duplicate) and incubated for 72h. At 72h the cells are treated with Promega Cell Titer Glo reagent for approximately 15 minutes. Luminescence (relative light units, RLU) is recorded using a BMG Polarstar Omega plate reader. The single agent concentration that results in a 50% reduction in total signal (IC₅₀) is calculated using PRISM software and a four-parameter non-linear regression curve fit. For combination studies the RLUs are plotted using PRISM on an XY plot with the gemcitabine concentration on the X axis and RLU on the Y axis. The RLU for each concentration of Chk1 inhibitor is plotted as a function of gemcitabine concentration. The IC50 for gemcitabine alone and at each concentration of Chk1 is determined using a four-parameter non-linear regression curve fit. The approximate concentration of Chk1 inhibitor that results in a two and ten-fold reduction in the IC₅₀ of gemcitabine alone is calculated as an indication of synergistic potency.

From the results obtained by following the above protocol, the IC₅₀ values against MIAPaca-2 cells of the compound of formula (1) alone (Chk1 IC₅₀), the approximate concentration of the compound that results in a two-fold (2×LS) and a 10-fold (10×LS) reduction in the IC₅₀ of gemcitabine alone of the compound of formula (1) are shown below.

| Chk1 IC₅₀ (nM) | 2 × LS (nM) | 10 × LS (nM) |
|---|---|---|
| 144 | 3 | 100 |

### Pharmaceutical Formulations

### (i) Tablet Formulation

A tablet composition containing a pharmaceutically acceptable salt as defined in any one of Embodiments 1.1 to 1.48 or the Examples above is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of pharmaceutically acceptable salt as defined in any one of Embodiments 1.1 to 1.48 or the Examples above with 100 mg lactose and filling the resulting mixture into standard opaque hard gelatin capsules.

(iii) Injectable Formulation I

A parenteral composition for administration by injection can be prepared by dissolving a pharmaceutically acceptable salt as defined in any one of Embodiments 1.1 to 1.48 or the Examples above in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a pharmaceutically acceptable salt as defined in any one of Embodiments 1.1 to 1.48 or the Examples above (2 mg/ml) and mannitol (50 mg/ml), sterile filtering the solution and filling into sealable 1 ml vials or ampoules.

### (v) Injectable Formulation III

A formulation for i.v. delivery by injection or infusion can be prepared by dissolving a pharmaceutically acceptable salt as defined in any one of Embodiments 1.1 to 1.48 or the Examples above in water at 20 mg/ml. The vial is then sealed and sterilised by autoclaving.

### (vi) Injectable Formulation IV

A formulation for i.v. delivery by injection or infusion can be prepared by dissolving a pharmaceutically acceptable salt as defined in any one of Embodiments 1.1 to 1.48 or the Examples above in water containing a buffer (e.g. 0.2 M acetate pH 4.6) at 20 mg/ml. The vial is then sealed and sterilised by autoclaving.

(vii) Subcutaneous Injection Formulation

A composition for sub-cutaneous administration is prepared by mixing a pharmaceutically acceptable salt as defined in any one of Embodiments 1.1 to 1.48 or the Examples above with pharmaceutical grade corn oil to give a concentration of 5 mg/ml. The composition is sterilised and filled into a suitable container.

(viii) Lyophilised Formulation

Aliquots of formulated a pharmaceutically acceptable salt as defined in any one of Embodiments 1.1 to 1.48 or the Examples above are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile which is selected from maleate, tosylate, besylate and malonate salts.

2. A pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile according to claim 1 which is a maleate salt.

3. A pharmaceutically acceptable salt according to claim 1 having a salt ratio (molar ratio of acid:free base) of approximately 1:1.

4. A pharmaceutically acceptable salt according to claim 1 which is from 50% to 100% crystalline.

5. A pharmaceutically acceptable maleate Pattern B salt of 5-[[5-[4-(4-fluoro-1-methyl-4-piperidyl)-2-methoxy-phenyl]-1H-pyrazol-3-yl]amino]pyrazine-2-carbonitrile according to claim 2 which has an XRPD spectrum characterised by major ° 2Th (° 2Theta) peaks at 6.9±0.2° and/or 26.4±0.2° and/or 11.8 0.2° and/or 17.9±0.2°.

6. A pharmaceutically acceptable maleate salt of 5-[[5-[4-(4-fluoro-1-methyl-4-piperidyl)-2-methoxy-phenyl]-1H-pyrazol-3-yl]amino]pyrazine-2-carbonitrile according to claim 2 which is a Pattern B salt having an XRPD spectrum substantially as shown in FIG. 25.

7. A pharmaceutical composition comprising a pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile as defined in claim 1, and a pharmaceutically acceptable excipient.

8. A method for the treatment of cancer, which method comprises administering to a patient a pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile as defined in claim 1.

9. A pharmaceutical combination comprising a pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile as defined in claim 1 and another therapeutically active agent.

10. A method of preparing a pharmaceutically acceptable salt as defined in claim 1; which process comprises dispersing 5-[[5-[4-(4-fluoro-1-methyl-4-piperidyl)-2-methoxy-phenyl]-1H-pyrazol-3-yl]amino]pyrazine-2-carbonitrile in tetrahydrofuran to form a mixture, heating the mixture to an elevated temperature in the range from 45° C. to 65° C. (e.g. from 55° C. to 65° C. and particularly approximately 60° C.), adding a required amount of an acid to the mixture; maintaining the mixture at or near the elevated temperature for a defined period and cooling the mixture to allow isolation of the pharmaceutically acceptable salt.

11. A method of preparing a pharmaceutically acceptable salt as defined in claim 1; which process comprises dispersing 5-[[5-[4-(4-fluoro-1-methyl-4-piperidyl)-2-methoxy-phenyl]-1H-pyrazol-3-yl]amino]pyrazine-2-carbonitrile in a mixture of tetrahydrofuran and acetonitrile (e.g. a 1:1 mixture) to form a mixture, heating the mixture to an elevated temperature in the range from 45° C. to 55° C. (e.g. approximately 50° C.), adding a required amount of an acid to the mixture; maintaining the mixture at or near the elevated temperature for a defined period and cooling the mixture to allow isolation of the pharmaceutically acceptable salt.

12. A method of preparing a pharmaceutically acceptable salt as defined in claim 1; which process comprises dispersing 5-[[5-[4-(4-fluoro-1-methyl-4-piperidyl)-2-methoxy-phenyl]-1H-pyrazol-3-yl]amino]pyrazine-2-carbonitrile in a mixture of tetrahydrofuran and water (e.g. wherein the mixture contains from 75% to 97% (v/v) tetrahydrofuran and from 3% to 25% (v/v) water, and more preferably approximately 95% (v/v) tetrahydrofuran and approximately 5 (v/v) water) to form a mixture, heating the mixture to an elevated temperature in the range from 45° C. to 65° C. (e.g. approximately 50° C. to 60° C.), adding a required amount of an acid to the mixture; maintaining the mixture at or near the elevated temperature for a defined period and cooling the mixture to allow isolation of the pharmaceutically acceptable salt.

13. A method according to claim 10 wherein the acid is maleic acid and the resulting pharmaceutically acceptable salt is a maleate salt.

14. A method according to claim 13 wherein the maleate salt is maleate salt Pattern A salt.

15. A method according to claim 14 which further comprises converting the Pattern A maleate salt to a Pattern B maleate salt by conditioning the Pattern A salt in an atmosphere of greater than 50% relative humidity.

16. (canceled)

17. A pharmaceutically acceptable salt according to claim 2 having a salt ratio (molar ratio of acid:free base) of approximately 1:1.

18. A pharmaceutically acceptable salt according to claim 2 which is from 50% to 100% crystalline.

19. A pharmaceutical composition comprising a pharmaceutically acceptable salt of 5-[[5-[4-(4-fluoro-1-methyl-4-piperidyl)-2-methoxy-phenyl]-1H-pyrazol-3-yl]amino]pyrazine-2-carbonitrile as defined in claim 2, and a pharmaceutically acceptable excipient.

20. A method according to claim 11 wherein the acid is maleic acid and the resulting pharmaceutically acceptable salt is a maleate salt.

21. A method according to claim 12 wherein the acid is maleic acid and the resulting pharmaceutically acceptable salt is a maleate salt.
